(19) United States
(12) Patent Application Publication
(10) Pub. No.: US 2025/0387335 A1
(43) Pub. Date: Dec. 25, 2025

(54) Pharmaceutical Compositions, and Preparation and Methods of Use Thereof

(71) Applicant: MAKScientific, LLC, Burlington, MA (US)

(72) Inventors: Alexandros Makriyannis, Watertown, MA (US); Kiran Vemuri, Boston, MA (US)

(21) Appl. No.: 18/835,417

(22) PCT Filed: Feb. 2, 2023
(86) PCT No.: PCT/US2023/061889

§ 371 Date: Aug. 2, 2024

Related U.S. Application Data
(60) Provisional application No. 63/306,005, filed on Feb. 2, 2022.

(51) Int. Cl. A61K 9/16; A61K 31/541

(52) U.S. Cl. CPC A61K 9/1652; A61K 31/541

(57) ABSTRACT

The invention relates to pharmaceutical compositions or formulations and comprises (I) a compound of Formula (I) or Formula (II); (ii) one or more pharmaceutically acceptable polymers; and (iii) optionally one or more pharmaceutically acceptable surfactants and/or one or more pharmaceutically acceptable surfactant-like materials; and (iv) optionally one or more pharmaceutically acceptable carriers and/or one or more pharmaceutically acceptable excipients; and (v) optionally one or more solvents, in varying ratios. The invention also discloses their preparation and methods of use thereof wherein the pharmaceutical compositions or formulations are administered in therapeutically effective amounts to provide a physiological effect in an individual or animal.

### STATEMENT REGARDING FEDERALLY SPONSORED RESEARCH

This invention was made with government support under R41DK115303 awarded by the National Institute of Diabetes and Digestive and Kidney Diseases, National Institute of Health. The government has certain rights in the invention.

### BACKGROUND

A majority of active pharmaceutical ingredients (APIs) in today's drug pipeline have low to no aqueous solubility which complicates their delivery and results in their poor bioavailability. Organic solvents, such as dimethyl sulfoxide (DMSO), are toxic and incompatible with clinical use; however, researchers often use them in early drug discovery and preclinical studies to assess their lead candidates. The challenge of poor solubility is often not properly addressed until late in the development process, which can create potential delays for clinical studies. To improve a drug's kinetic solubility, formulators commonly use amorphous solid dispersions (ASD, wherein amorphous API is combined with a polymer and optionally other excipients including surfactants). Techniques to convert a crystalline API to amorphous form include mixing with polymers, surfactants and other pharmaceutically acceptable carriers, excipients and/or solvents (collectively referred to as “matrix”) and apply these formulations towards spray dried dispersion (SDD) and hot melt extrusion (HME) technologies and further towards pharmaceutically acceptable human dosage forms. ASDs typically comprise of an amorphous API dispersed in a polymer matrix. Polymers in ASDs disarrange the crystalline lattice of the API and produce a higher energy amorphous state which exhibits higher dissolution rate, solubility, and bioavailability. The polymers also prevent the recrystallization of the drug, maintain drug supersaturation and provide improved physical stability of API in accelerated temperature and humidity conditions which increases the overall shelf-life of the drug product. The technique of (ASD) is being widely used to overcome these issues due to its easily scalable manufacturing process and flexibility and control this technique offers to develop an optimized drug product.

One of the main challenges of the ASD formulation development is the choice of polymer matrix.

Polymer matrix choice is driven by maximum miscibility of the API and polymer.

SDD has been commonly used to produce amorphous solid dosage forms for improving the bioavailability of poorly soluble crystalline API. The process provides exceptional flexibility for manufacturing because many different solvents, polymer mixtures, solute concentrations, process temperatures, and atomization pressures can be used, depending on the API while providing reproducible and uniform control over the process and the yielded SDD. The primary purpose of spray drying poorly soluble crystalline APIs is to achieve an amorphous molecularly dispersed state of the API in the matrix of choice.

Among the numerous suggested approaches, HME has been established as an efficient technology for the enhancement of solubility and bioavailability of poorly soluble drugs. The SWOT analysis of HME suggests significant advantages of this platform as compared to other techniques. Regardless of gaining popularity, HME processing may induce thermal degradation of API and carrier at certain processing temperatures, as well as recrystallization of API during storage of the HME product. However, these issues are addressed by reducing the processing temperatures by use additives such as polymers as plasticizers, reduction of residence time of materials during extrusion and proper selection of polymer carriers. The use of various process analytical technology tools coupled with HME represents promising manufacturing methods and for the successful development of pharmaceutical products. During the melt extrusion process, the dissolution of APIs into the polymer matrix is accelerated under the influence of shear and heat. The amorphous solid dispersions produced via HME are expected to possess lower molecular mobilities and API molecules “freeze” inside a matrix of choice to inhibit the nucleation and crystallization processes.

To accomplish the above said processes, determining the right polymer and/or combinations of polymer and surfactant along with other additives is of high significance and is novel. Moreover, the combinations are important for the API to exhibit a stable shelf-life while being non-crystalline in form. The ability to address solubility issues early can save costs and time. Additionally, such novel pharmaceutical combinations will ensure their ability to be pharmaceutically acceptable, not be toxic, and be therapeutically effective without negatively influencing the overall the potency and efficacy of the active drug form.

Particularly, the technology would be advantageous for formulating and developing cannabinoid-based medications for treating various diseases relating to the lung, liver, kidney and prostate. Moreover, cannabinoid receptor-mediated signaling has emerged as a novel signaling pathway regulating inflammatory conditions and fibrogenesis wherein both cannabinoid receptor-1 (CB1) and cannabinoid receptor-2 (CB2) have been implicated in disease-states pertaining to the liver (1-10), lung (11-13), renal (14-18) and prostate-related (19-22) proliferative diseases.

### REFERENCES

1. CB1 cannabinoid receptor antagonism: a new strategy for the treatment of liver fibrosis. Nat Med. 2006; 12:671-676.
2. Role of cannabinoid receptors in alcoholic hepatic injury: steatosis and fibrogenesis are increased in CB2 receptor-deficient mice and decreased in CB1 receptor knockouts. Liver Int. 2011 July; 31(6):860-70.
3. Role of cannabinoid receptors in alcoholic hepatic injury: steatosis and fibrogenesis are increased in CB2 receptor-deficient mice and decreased in CB1 receptor knockouts. Liver Int. 2011; 31:860-870.
4. Cannabinoid receptor type I modulates alcohol-induced liver fibrosis. Mol Med. 2011; 17(11-12):1285-94.
5. Cannabinoid receptor type I modulates alcohol-induced liver fibrosis. Mol Med. 2011; 17:1285-1294.
6. Reversal of liver fibrosis by the antagonism of endocannabinoid CB1 receptor in a rat model of CCI(4)-induced advanced cirrhosis. Lab Invest. 2012; 92(3):384-95.
7. Prevention of fibrosis progression in CCI4-treated rats: role of the hepatic endocannabinoid and apelinsystems. J Pharmacol Exp Ther. 2012; 340:629-637.
8. Design, Synthesis, and Biological Evaluation of Novel, Non-Brain-Penetrant, Hybrid Cannabinoid CB1R Inverse Agonist/Inducible Nitric Oxide Synthase (iNOS) Inhibitors for the Treatment of Liver Fibrosis. J Med Chem. 2017; 60(3):1126-1141.
9. The peripheral CB1 receptor antagonist JD5037 attenuates liver fibrosis via a CB1 receptor/β-arrestin1/Akt pathway. Br J Pharmacol. 2020; 177(12):2830-2847.
10. Peripheral Hybrid CB1R and iNOS Antagonist MRI-1867 Displays Anti-Fibrotic Efficacy in Bleomycin-Induced Skin Fibrosis. Front Endocrinol (Lausanne). 2021 28; 12:744857.
11. Endocannabinoid signalling/cannabinoid receptor 2 is involved in icariin-mediated protective effects against bleomycin-induced pulmonary fibrosis. Phytomedicine. 2022; 103:154187.
12. Cannabinoid CB1 receptor overactivity contributes to the pathogenesis of idiopathic pulmonary fibrosis. JCI Insight. 2017; 2(8):e92281.
13. Protection from Radiation-Induced Pulmonary Fibrosis by Peripheral Targeting of Cannabinoid Receptor-1. Am J Respir Cell Mol Biol. 2015; 53(4):555-62.
14. Cannabinoid receptor 1 disturbance of PPARγ2 augments hyperglycemia induction of mesangial inflammation and fibrosis in renal glomeruli. J Mol Med (Berl) 2014; 92:779-792.
15. Targeted inhibition of the type 2 cannabinoid receptor is a novel approach to reduce renal fibrosis. Kidney Int. 2018; 94(4):756-772.
16. (+)-trans-Cannabidiol-2-hydroxy pentyl is a dual CB1R antagonist/CB2R agonist that prevents diabetic nephropathy in mice. Pharmacol Res. 2021 July; 169:105492.
17. Cannabinoid receptor type 2 promotes kidney fibrosis through orchestrating β-catenin signaling. Kidney Int. 2021 February; 99(2):364-381.
18. Interference with TGFβ1-Mediated Inflammation and Fibrosis Underlies Reno-Protective Effects of the CB1 Receptor Neutral Antagonists AM6545 and AM4113 in a Rat Model of Metabolic Syndrome. Molecules. 2021; 26(4):866.
19. Expression of functionally active cannabinoid receptor CB 1 in the human prostate gland: Cannabinoid Receptors in Prostate Gland. The Prostate. 2003; 54:95-102.
20. Use of a Cannabinoid CB1 Receptor Antagonist for Preparation of Drugs Useful for the Prevention and Treatment of Benign Prostatic Hypertrophy. WO2007141413A1.
21. Transient Receptor Potential A1 and Cannabinoid Receptor Activity in Human Normal and Hyperplastic Prostate: Relation to Nerves and Interstitial Cells. Eur Urol. 2010; 57:902-910.
22. Proapoptotic effect of endocannabinoids in prostate cancer cells. Oncol Rep. 2015; 33(4):1599-608.
23. The Endocannabinoid System: A Target for Cancer Treatment. 2020; Int J Mol Sci 21:747.

### BRIEF DESCRIPTION OF DRAWINGS

FIG. 1 shows the solubility results for Compound 1 alone, compared to polymer matrices thereof;

FIG. 2 shows the solubility results for Compound 1 (API) alone compared to polymer matrices or polymer matrices combined with surfactants;

FIG. 3 shows the solubility results for Compound 1 alone, compared to polymer matrices thereof combined with surfactants;

FIG. 4 shows the solubility results for Compound 1 (API) alone, compared to polymer matrices combined with surfactants;

FIG. 5 shows X-ray diffraction patterns of Compound 1 alone showing crystalline structure;

FIG. 6 shows X-ray diffraction patterns of Compound 1 as dispersion in a polymer (HPMCAS-L) in a Compound 1:polymer ratio of 10:90;

FIG. 7 shows X-ray diffraction patterns of Compound 1 as dispersion in a polymer (HPMC HP55) in a Compound 1:polymer ratio of 25:75;

FIG. 8 shows X-ray diffraction patterns of Compound 1 as dispersion in a polymer (HPMC HP55)+surfactant (Poloxamer P407) in a Compound 1:polymer:surfactant ratio of 25:65:10;

FIG. 9 shows the solubility results for Compound 2 alone, compared to polymer matrices thereof;

FIG. 10 shows the solubility results for Compound 2 alone, compared to a polymer matrix thereof and polymer matrices thereof combined with surfactants;

FIG. 11 shows X-ray diffraction patterns of Compound 2 alone, showing crystalline structure;

FIG. 12 shows X-ray diffraction patterns of Compound 2 as dispersion in a polymer (HPMC HP55)+surfactant (Poloxamer P407) in a Compound 2:polymer:surfactant ratio of 25:65:10;

FIG. 13 shows differential scanning calorimetry patterns of Compound 2 alone;

FIG. 14 shows Differential scanning calorimetry patterns of Compound 2 as dispersion in a polymer (HPMC HP55)+surfactant (Poloxamer P407) in a Compound 2:polymer:surfactant ratio of 25:65:10;

FIG. 15 shows the mouse PK study results for Compound 1 alone and in combination with polymer+surfactant;

FIG. 16 shows data for a mouse PK study for Compound 1 alone and in combination with different polymer+surfactant from that of FIG. 15;

FIG. 17 shows the mouse PK study results for Compound 2 alone or in combination with polymer+surfactant;

FIG. 18 shows the structures of Compound 1 and Compound 2 as referenced herein;

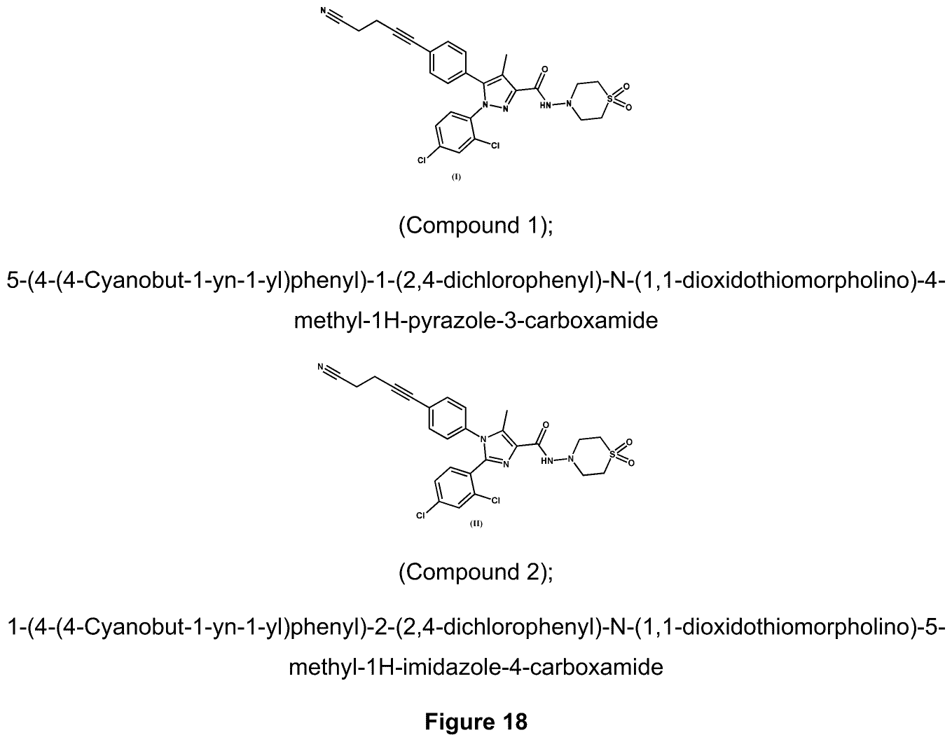

FIG. 19 shows a Table 1 with pharmacokinetic parameters for Compound 1 alone and in combination with polymer+surfactant;

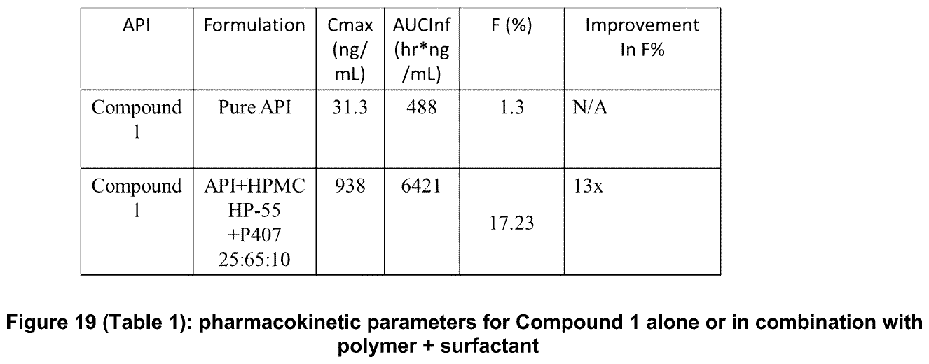

FIG. 20 shows a Table 2 with pharmacokinetic parameters for Compound 1 alone or in combination with a polymer+surfactant that is different from that of Table 1; and

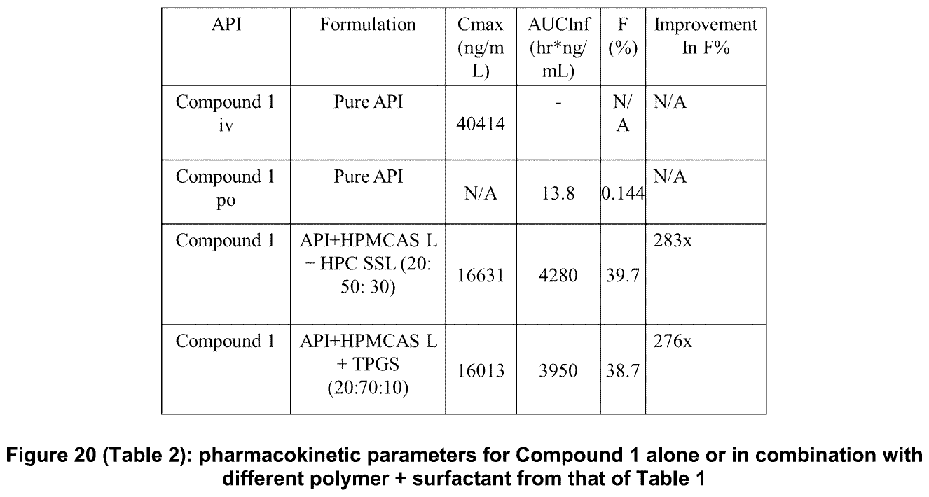

FIG. 21 shows a Table 3 with pharmacokinetic parameters for Compound 2 alone or in combination with polymer+surfactant.

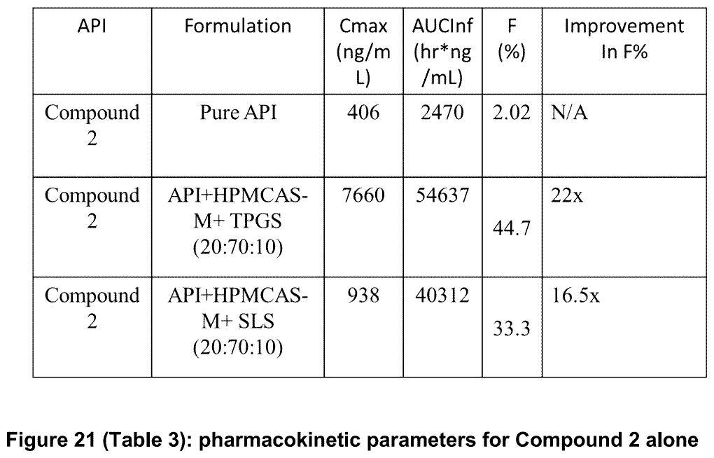

### SUMMARY OF THE INVENTION

The present invention describes novel pharmaceutical compositions or formulations comprising combinations of a compound as the active pharmaceutical ingredients (API), polymers and optionally surfactants, in varying ratios that can be scaled up towards manufacturing and towards making Spray Dried Dispersions (SDD) and/or extrudates (Hot melt Extrusion) and ultimately towards human dosage forms. These pharmaceutical compositions or formulations may be combined with other solubilizing techniques involving pharmaceutically acceptable carriers, excipients including oily vehicles, co-surfactants and/or solvents for transforming active pharmaceutical ingredients into clinically usable human dosage forms. Especially, the pharmaceutical compositions comprise cannabinoid modulators, to be studied in animals and for their use in humans. In some embodiments, the cannabinoid modulators are peripherally acting CB1 or CB2 antagonists that can be formulated along with a pharmaceutically acceptable polymer and optionally a pharmaceutically acceptable surfactant to enhance the spring-parachute effect and improve the overall solubility and bioavailability of the compound. In certain embodiments, the novel pharmaceutical compositions can be used to treat fibrotic and related inflammatory conditions of the lung, liver, kidney and prostate in humans including renal, hepatic and pulmonary fibrosis.

Briefly stated, an embodiment of the invention is concerned with a pharmaceutical composition or formulation, comprising: (i) a compound of Formula I or Formula II:

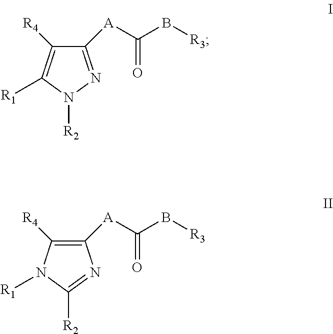

(ii) one or more pharmaceutically acceptable polymers;
(iii) optionally one or more pharmaceutically acceptable surfactants and/or one or more pharmaceutically acceptable surfactant-like materials;
(iv) optionally one or more pharmaceutically acceptable carriers and/or one or more pharmaceutically acceptable excipients; and
(v) optionally one or more solvents, in varying ratios, wherein
   A is a direct bond,
B is N(H),
R1 is -T-(CH₂)ₘ-Q-(CH₂)ₙ—Z,
each of m and n is independently an integer from 0 to about 7;
T is selected from the group consisting of a carbocyclic ring having 3 to about 8 ring members, an unsaturated ring having 3 to about 8 carbon atoms as ring members, an aromatic ring having 5 to about 8 carbon atoms as ring members, a heterocyclic ring having 3 to about 8 ring members, a heteroaromatic ring having 5 to about 8 ring members, a bicyclic ring, a heterobicyclic ring, a tricyclic ring, a heterotricyclic ring, a polycyclic ring or a heteropolycyclic ring;
Q is CH═CH or C≡C;
Z is selected from the group consisting of H, halogen, CF₃, CF₂H, N₃, NCS, CN, NO₂, NX₁X₂, OX₃, SX₃, OAc, OSO₂X₃, O-acyl, S-acyl, SO₂-alkyl, SO-alkyl, SC(CH₃)₂COOX₈, OC(CH₃)₂COOX₈, C(CH₃)₂COOX₈, Si(alkyl)₃, alkyl-CN, O-aroyl, O(CH₂)ⱼOX₃, O(CH₂)ⱼNX₁X₂, NH-acyl, NH-aroyl, CHO, C(halogen)₃, COOX₃, SO₃H, SO₂NX₁X₂, CONX₁X₂, NHC(O)O-alkyl, NHSO2-alkyl, alkoxy, alkyl, alcohol, alkylmercapto, alkylamino, di-alkylamino, alkylsulfinyl, alkylsulfonyl, CX₄X₅X₆, —CH═CHX₈, and —C≡CX₈;
each of X₁ and X₂ is independently H or alkyl, or
X₁ and X₂ together form part of a heterocyclic ring having about 4 to about 7 ring members and optionally one additional heteroatom selected from O, N and S, or
X₁ and X₂ together form part of an imide ring having about 5 to about 6 members,
X₃ is selected from the group consisting of H, alkyl, NO₂, (CH₂)ₘCN, hydroxyloweralkyl, and alkyl-NX₁X₂,
each of X₄, X₅, and X₆ is independently selected from the group consisting of H, alkyl, carbocyclic ring, hydroxyloweralkyl, alkyl-OH, halogen, CN, SNO, S(SO₂)alkyl, NX₁X₂, COOX₃, CONX₃, OX₇, and O-alkyl-X₇, wherein
X₇ is selected from the group consisting of H, alkyl, NO₂, NO, P(O)(OX₈)₂, PH(O)(OX₈), S(O)ₖN(alkyl)₂, S(O)ₖX₈, S(O)ₖOX₈, COOX₈, CONX₈, SO₃H and COX₈, wherein
X₈ is selected from the group consisting of H, alkyl, carbocyclic ring, heterocyclic ring, aromatic ring, heteroaromatic ring and —CX₉═CHX₁₀,
each of X₉ and X₁₀ is independently H or alkyl,
m is an integer from 0 to 7,
j is an integer from 0 to about 6, and
k is an integer from 0 to about 2;
R1 is -T-(CH₂)ₘ-Q-(CH₂)ₙ—Z;
each of m and n is independently an integer from 0 to about 7;
T is selected from the group consisting of a carbocyclic ring having 3 to about 8 ring members, an unsaturated ring having 3 to about 8 carbon atoms as ring members, an aromatic ring having 5 to about 8 carbon atoms as ring members, a heterocyclic ring having 3 to about 8 ring members, a heteroaromatic ring having 5 to about 8 ring members, a bicyclic ring, a heterobicyclic ring, a tricyclic ring, a heterotricyclic ring, a polycyclic ring and a heteropolycyclic ring;
Q is CH═CH or C≡C; and
Z is selected from the group consisting of a carbocyclic ring having about 4 to about 7 ring members, a heterocyclic ring having about 4 to about 7 ring members, an aromatic ring having about 5 to about 7 ring members, a heteroaromatic ring having about 5 to about 7 ring members, a bicyclic ring, a heterobicyclic ring, a tricyclic ring, a heterotricyclic ring, a polycyclic ring, a heteropolycyclic ring; or any above group substituted on at least one available ring atom by an alkyl group; or any above group substituted on at least one available ring nitrogen atom by a benzyl group, a substituted benzyl group, an alkoxybenzyl group, a substituted alkoxybenzyl group, a benzhydryl group or a substituted benzhydryl group; and wherein the connecting point between the —(CH₂)ₙ— group and the Z group can be any available ring carbon atom or any available ring nitrogen atom; or
R1 is -T-(CH₂)ₘ-Q-(CH₂)ₙ—Z;
each of m and n is independently an integer from 0 to about 7;
T is selected from the group consisting of a carbocyclic ring having 3 to about 8 ring members, an unsaturated ring having 3 to about 8 carbon atoms as ring members, an aromatic ring having 5 to about 8 carbon atoms as ring members, a heterocyclic ring having 3 to about 8 ring members, a heteroaromatic ring having 5 to about 8 ring members, a bicyclic ring, a heterobicyclic ring, a tricyclic ring, a heterotricyclic ring, a polycyclic ring and a heteropolycyclic ring;
Q is CH═CH or C≡C; and
Z is selected from the group consisting of 1-, 2- or 3-pyrrolidinyl, 1-, 2-, 3- or 4-piperidinyl, 2-, 3- or 4-morpholinyl, 2-, 3- or 4-thiomorpholinyl, 1-, 2- or 3-azetidinyl, 1- or 2-piperazinyl, 2- or 3-tetrahydrofuranyl; or any above group substituted on at least one available ring atom by an alkyl group; or any above group substituted on at least one available ring nitrogen atom by a benzyl group, a substituted benzyl group, an alkoxybenzyl group, a substituted alkoxybenzyl group, a benzhydryl group or a substituted benzhydryl group; and wherein the connecting point between the —(CH₂)ₙ— group and the Z group can be any available ring carbon atom or any available ring nitrogen atom; or
R1 is -T-(CH₂)ₘ-Q-(CH₂)ₙ—Z;
each of m and n is independently an integer from 0 to about 7;
T is selected from the group consisting of a carbocyclic ring having 3 to about 8 ring members, an unsaturated ring having 3 to about 8 carbon atoms as ring members, an aromatic ring having 5 to about 8 carbon atoms as ring members, a heterocyclic ring having 3 to about 8 ring members, a heteroaromatic ring having 5 to about 8 ring members, a bicyclic ring, a heterobicyclic ring, a tricyclic ring, a heterotricyclic ring, a polycyclic ring and a heteropolycyclic ring;
Q is CH═CH or C≡C; and
Z is selected from the group consisting of

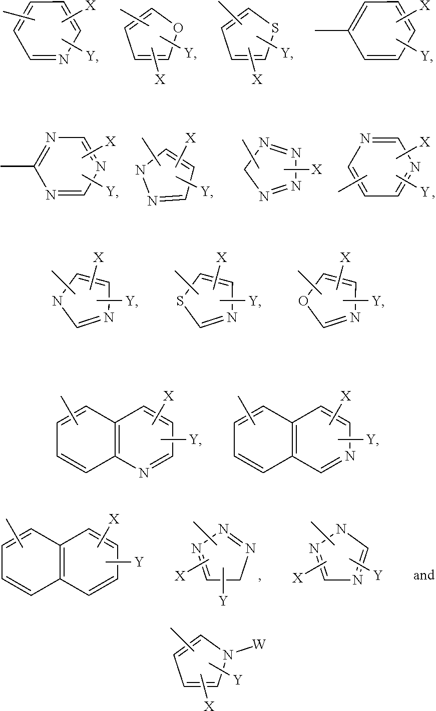

wherein each of X and Y is independently selected from the group consisting of H, halogen, CF₃, CF₂H, N₃, NCS, CN, NO₂, NX₁X₂, OX₃, SX₃, OAc, OSO₂X₃, O-acyl, S-acyl, SO₂-alkyl, SO-alkyl, SC(CH₃)₂COOX₈, OC(CH₃)₂COOX₈, C(CH₃)₂COOX₈, Si(alkyl)₃, alkyl-CN, O-aroyl, O(CH₂)ⱼOX₃, O(CH₂)ⱼNX₁X₂, NH-acyl, NH-aroyl, CHO, C(halogen)₃, COOX₃, SO₃H, SO₂NX₁X₂, CONX₁X₂, NHC(O)O-alkyl, NHSO2-alkyl, alkoxy, alkyl, alcohol, alkylmercapto, alkylamino, di-alkylamino, alkylsulfinyl or alkylsulfonyl, CX₄X₅X₆, —CH═CHX₈, and —C≡CX₈;
each of X₁ and X₂ is independently H or alkyl, or
X₁ and X₂ together form part of a heterocyclic ring having about 4 to about 7 ring members and optionally one additional heteroatom selected from O, N and S, or
X₁ and X₂ together form part of an imide ring having about 5 to about 6 members,
X₃ is selected from the group consisting of H, alkyl, NO₂, (CH₂)ₘCN, hydroxyloweralkyl, and alkyl-NX₁X₂, each of X₄, X₅, and X₆ is independently selected from H, alkyl, carbocyclic ring, hydroxyloweralkyl, alkyl-OH, halogen, CN, SNO, S(SO₂)alkyl, NX₁X₂, COOX₃, CONX₃, OX₇, and O-alkyl-X₇, wherein
X₇ is selected from the group consisting of H, alkyl, NO₂, NO, P(O)(OX₈)₂, PH(O)(OX₈), S(O)ₖN(alkyl)₂, S(O)ₖX₈, S(O)ₖOX₈, COOX₈, CONX₈, SO₃H, and COX₈, wherein
X₈ is selected from the group consisting of H, alkyl, carbocyclic ring, heterocyclic ring, aromatic ring, heteroaromatic ring, and —CX₉═CHX₁₀, wherein
each of X₉ and X₁₀ is independently H or alkyl, wherein m is an integer from 0 to 7;
j is an integer from 0 to about 6,
k is an integer from 0 to about 2,
W is H or alkyl; or
R1 is-T (CH₂)ₘ-Q-(CH₂)ₙ—Z;
each of m and n is independently an integer from 0 to about 7;
T is selected from the group consisting of a carbocyclic ring having 3 to about 8 ring members, an unsaturated ring having 3 to about 8 carbon atoms as ring members, an aromatic ring having 5 to about 8 carbon atoms as ring members, a heterocyclic ring having 3 to about 8 ring members, a heteroaromatic ring having 5 to about 8 ring members, a bicyclic ring, a heterobicyclic ring, a tricyclic ring, a heterotricyclic ring, a polycyclic ring and a heteropolycyclic ring;
Q is CH═CH or C≡C; and
Z is selected from the group consisting of an unsaturated ring having 5 ring atoms and 0 to 2 independently selected heteroatoms as ring members fused to an unsaturated ring having 5 ring atoms and 0 to 4 independently selected heteroatoms as ring members, an unsaturated ring having 5 ring atoms and 0 to 2 independently selected heteroatoms as ring members fused to an unsaturated ring having 6 or 7 ring atoms and 0 to 4 independently selected heteroatoms as ring members or an unsaturated ring having 6 ring atoms and 0 to 3 independently selected heteroatoms as ring members fused to an unsaturated ring having 6 or 7 ring atoms and 0 to 4 independently selected heteroatoms as ring members; or
R1 is -T-(CH₂)ₘ-Q (CH₂)ₙ—Z;
T is selected from the group consisting of a carbocyclic ring having 3 to about 8 ring members, an unsaturated ring having 3 to about 8 carbon atoms as ring members, an aromatic ring having 5 to about 8 carbon atoms as ring members, a heterocyclic ring having 3 to about 8 ring members, a heteroaromatic ring having 5 to about 8 ring members, a bicyclic ring, a heterobicyclic ring, a tricyclic ring, a heterotricyclic ring, a polycyclic ring and a heteropolycyclic ring;
each of m and n is independently an integer from 0 to about 7;
Q is CH═CH or C≡C;
Z is

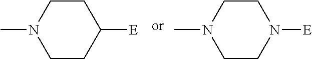

E is selected from the group consisting of a C1 to about C4, linear or branched alkyl group, a phenyl group, a substituted phenyl group, a benzyl group and a substituted benzyl group; or
R1 is -T-(CH₂)ₘ-Q-(CH₂)ₙ—Z;
T is selected from the group consisting of a carbocyclic ring having 3 to about 8 ring members, an unsaturated ring having 3 to about 8 carbon atoms as ring members, an aromatic ring having 5 to about 8 carbon atoms as ring members, a heterocyclic ring having 3 to about 8 ring members, a heteroaromatic ring having 5 to about 8 ring members, a bicyclic ring, a heterobicyclic ring, a tricyclic ring, a heterotricyclic ring, a polycyclic ring and a heteropolycyclic ring;
each of m and n is independently an integer from 0 to about 7;
Q is CH═CH or C≡C; and
Z is selected from the group consisting of

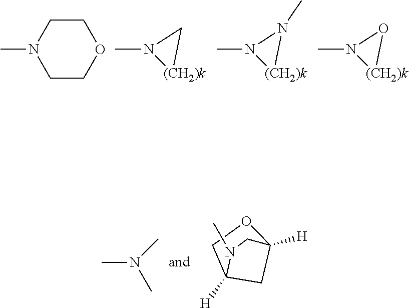

k is an integer from 1 to about 5, and
each of A₁ and A₂ is independently selected from the group consisting of a C1 to about C4 alkyl group, a phenyl group or a substituted phenyl group; or
R1 is —(CH₂)ₙ—Z;
n is an integer from 0 to about 7; wherein
Z is selected from the group consisting of H, halogen, CF₃, CF₂H, N₃, NCS, CN, NO₂, NX₁X₂, OX₃, SX₃, OAc, OSO₂X₃, O-acyl, S-acyl, SO₂-alkyl, SO-alkyl, SC(CH₃)₂COOX₈, OC(CH₃)₂COOX₈, C(CH₃)₂COOX₈, Si(alkyl)₃, alkyl-CN, O-aroyl, O(CH₂)ⱼOX₃, O(CH₂)ⱼNX₁X₂, NH-acyl, NH-aroyl, CHO, C(halogen)₃, COOX₃, SO₃H, SO₂NX₁X₂, CONX₁X₂, NHC(O)O-alkyl, NHSO2-alkyl, alkoxy, alkyl, alcohol, alkylmercapto, alkylamino, di-alkylamino, alkylsulfinyl or alkylsulfonyl, CX₄X₅X₆, —CH═CHX₈, and —C≡CX₈;
each of X₁ and X₂ is independently H or alkyl, or
X₁ and X₂ together form part of a heterocyclic ring having about 4 to about 7 ring members and optionally one additional heteroatom selected from O, N and S, or
X₁ and X₂ together form part of an imide ring having about 5 to about 6 members,
X₃ is selected from the group consisting of H, alkyl, aryl, NO₂, (CH₂)ₘCN, hydroxyloweralkyl, and alkyl-NX₁X₂,
each of X₄, X₅, and X₆ is independently selected from the group consisting of H, alkyl, carbocyclic ring, hydroxyloweralkyl, alkyl-OH, halogen, CN, SNO, S(SO₂)alkyl, NX₁X₂, COOX₃, CONX₃, OX₇, and O-alkyl-X₇, wherein
X₇ is selected from the group consisting of H, alkyl, NO₂, NO, P(O)(OX₈)₂, PH(O)(OX₈), S(O)ₖN(alkyl)₂, S(O)ₖX₈, S(O)ₖOX₈, COOX₈, CONX₈, SO₃H, and COX₈, wherein
X₈ is selected from the group consisting of H, alkyl, carbocyclic ring, heterocyclic ring, aromatic ring, heteroaromatic ring, and —CX₉═CHX₁₀, wherein
each of X₉ and X₁₀ is independently H or alkyl,
wherein m is an integer from 0 to 7,
j is an integer from 0 to about 6, or
k is an integer from 0 to about 2; or
R1 is —(CH₂)ₙ—Z;
n is an integer from 0 to about 7; and
Z is selected from the group consisting of a carbocyclic ring having about 4 to about 7 ring members, a heterocyclic ring having about 4 to about 7 ring members, an aromatic ring having about 5 to about 7 ring members, a heteroaromatic ring having about 5 to about 7 ring members, a bicyclic ring, a heterobicyclic ring, a tricyclic ring, a heterotricyclic ring, a polycyclic ring, a heteropolycyclic ring; or any above group substituted on at least one available ring atom by an alkyl group; or any above group substituted on at least one available ring nitrogen atom by a benzyl group, a substituted benzyl group, an alkoxybenzyl group, a substituted alkoxybenzyl group, a benzhydryl group or a substituted benzhydryl group; and wherein the connecting point between the —(CH₂)ₙ— group and the Z group can be any available ring carbon atom or any available ring nitrogen atom; or
R1 is —(CH₂)ₙ—Z;
n is an integer from 0 to about 7; and
Z is selected from the group consisting of a 5 member unsaturated ring having 0 to 4 independently selected heteroatoms as ring members, a substituted 5 member unsaturated ring having 0 to 4 independently selected heteroatoms as ring members, a 6 member aromatic ring having 0 to 5 independently selected heteroatoms as ring members or a substituted 6 member aromatic ring having 0 to 5 independently selected heteroatoms; and wherein the connecting point between the —(CH₂)ₙ— group and the Z group can be any available ring carbon atom or any available ring nitrogen atom; or
R1 is —(CH₂)ₙ—Z;
n is an integer from 0 to about 7; and
Z is selected from the group consisting of 1-, 2- or 3-pyrrolidinyl, 1-, 2-, 3- or 4-piperidinyl, 2-, 3- or 4-morpholinyl, 2-, 3- or 4-thiomorpholinyl, 1-, 2- or 3-azetidinyl, 1- or 2-piperazinyl, 2- or 3-tetrahydrofuranyl; or any above group substituted on at least one available ring atom by an alkyl group; or any above group substituted on at least one available ring nitrogen atom by a benzyl group, a substituted benzyl group, an alkoxybenzyl group, a substituted alkoxybenzyl group, a benzhydryl group or a substituted benzhydryl group; and wherein the connecting point between the —(CH₂)ₙ— group and the Z group can be any available ring carbon atom or any available ring nitrogen atom; or
R1 is —(CH₂)ₙ—Z;
n is an integer from 0 to about 7; and
Z is selected from the group consisting of

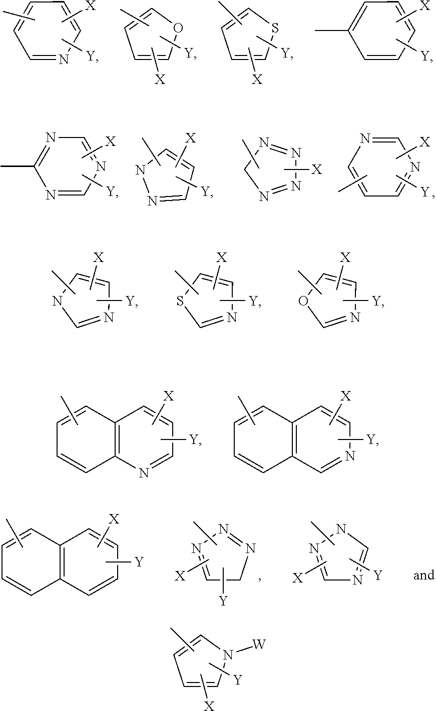

wherein each of X and Y is independently selected from the group consisting of, H, halogen, CF₃, CF₂H, N₃, NCS, CN, NO₂, NX₁X₂, OX₃, SX₃, OAc, OSO₂X₃, O-acyl, S-acyl, SO₂-alkyl, SO-alkyl, SC(CH₃)₂COOX₈, OC(CH₃)₂COOX₈, C(CH₃)₂COOX₈, Si(alkyl)₃, alkyl-CN, O-aroyl, O(CH₂)ⱼOX₃, O(CH₂)ⱼNX₁X₂, NH-acyl, NH-aroyl, CHO, C(halogen)₃, COOX₃, SO₃H, SO₂NX₁X₂, CONX₁X₂, NHC(O)O-alkyl, NHSO2-alkyl, alkoxy, alkyl, alcohol, alkylmercapto, alkylamino, di-alkylamino, alkylsulfinyl or alkylsulfonyl, CX₄X₅X₆, —CH═CHX₈, and —C≡CX₈;
each of X₁ and X₂ is independently H or alkyl, or
X₁ and X₂ together form part of a heterocyclic ring having about 4 to about 7 ring members and optionally one additional heteroatom selected from O, N and S, or
X₁ and X₂ together form part of an imide ring having about 5 to about 6 members,
X₃ is selected from the group consisting of H, alkyl, aryl, NO₂, (CH₂)ₘCN, hydroxyloweralkyl, and alkyl-NX₁X₂,
each of X₄, X₅, and X₆ is independently selected from H, alkyl, carbocyclic ring, hydroxyloweralkyl, alkyl-OH, halogen, CN, SNO, S(SO₂)alkyl, NX₁X₂, COOX₃, CONX₃, OX₇, and O-alkyl-X₇, wherein
X₇ is selected from the group consisting of H, alkyl, NO₂, NO, P(O)(OX₈)₂, PH(O)(OX₈), S(O)ₖN(alkyl)₂, S(O)ₖX₈, S(O)ₖOX₈, COOX₈, CONX₈, SO₃H, and COX₈, wherein
X₈ is selected from the group consisting of H, alkyl, carbocyclic ring, heterocyclic ring, aromatic ring, heteroaromatic ring, and —CX₉═CHX₁₀, wherein
each of X₉ and X₁₀ is independently H or alkyl,
wherein m is an integer from 0 to 7,
j is an integer from 0 to about 6, or
k is an integer from 0 to about 2
W is H or alkyl; or
R1 is —(CH₂)ₙ—Z;
n is an integer from 0 to about 7; and
Z is selected from the group consisting of a carbocyclic ring having 6 ring atoms fused to a heterocyclic ring having from 5 to 7 ring atoms, a carbocyclic ring having 6 ring atoms fused to a heteroaromatic ring having from 5 to 7 ring atoms, a heterocyclic ring having 6 ring atoms fused to a heterocyclic ring having from 5 to 7 ring atoms, an heterocyclic ring having 6 ring atoms fused to a heteroaromatic ring having from 5 to 7 ring atoms, an aromatic ring having 6 ring atoms fused to a heterocyclic ring having from 5 to 7 ring atoms, an aromatic ring having 6 ring atoms fused to a heteroaromatic ring having from 5 to 7 ring atoms, a heteroaromatic ring having 6 ring atoms fused to a heterocyclic ring having from 5 to 7 ring atoms or a heteroaromatic ring having 6 ring atoms fused to a heteroaromatic ring having from 5 to 7 ring atoms; or
R1 is —(CH₂)ₙ—Z;
n is an integer from 0 to about 7; and
Z is selected from the group consisting of an unsaturated ring having 5 ring atoms and 0 to 2 independently selected heteroatoms as ring members fused to an unsaturated ring having 5 ring atoms and 0 to 4 independently selected heteroatoms as ring members, an unsaturated ring having 5 ring atoms and 0 to 2 independently selected heteroatoms as ring members fused to an unsaturated ring having 6 or 7 ring atoms and 0 to 3 independently selected heteroatoms as ring members or an unsaturated ring having 6 ring atoms and 0 to 4 independently selected heteroatoms as ring members fused to an unsaturated ring having 6 or 7 ring atoms and 0 to 4 independently selected heteroatoms as ring members; or
R1 is —(CH₂)ₘ-Q₁-(CH₂)ₙ—Z;
Q₁ is selected from the group consisting of NH, O, S, —CH═CH—, —C≡C—, —CO, SO₂ and OSO₂;
m is an integer from 1 to about 7;
n is an integer from 0 to about 7; and
wherein Z is selected from the group consisting of H, halogen, CF₃, CF₂H, N₃, NCS, CN, NO₂, NX₁X₂, OX₃, SX₃, OAc, OSO₂X₃, O-acyl, S-acyl, SO₂-alkyl, SO-alkyl, SC(CH₃)₂COOX₈, OC(CH₃)₂COOX₈, C(CH₃)₂COOX₈, Si(alkyl)₃, alkyl-CN, O-aroyl, O(CH₂)ⱼOX₃, O(CH₂)ⱼNX₁X₂, NH-acyl, NH-aroyl, CHO, C(halogen)₃, COOX₃, SO₃H, SO₂NX₁X₂, CONX₁X₂, NHC(O)O-alkyl, NHSO2-alkyl, alkoxy, alkyl, alcohol, alkylmercapto, alkylamino, di-alkylamino, alkylsulfinyl or alkylsulfonyl, CX₄X₅X₆, —CH═CHX₈, and —C≡CX₈;
each of X₁ and X₂ is independently H or alkyl, or
X₁ and X₂ together form part of a heterocyclic ring having about 4 to about 7 ring members and optionally one additional heteroatom selected from O, N and S, or
X₁ and X₂ together form part of an imide ring having about 5 to about 6 members,
X₃ is selected from the group consisting of H, alkyl, aryl, NO₂, (CH₂)ₘCN, hydroxyloweralkyl, and alkyl-NX₁X₂,
X₄, X₅, and X₆ is independently selected from the group consisting of H, alkyl, carbocyclic ring, hydroxyloweralkyl, alkyl-OH, halogen, CN, SNO, S(SO₂)alkyl, NX₁X₂, COOX₃, CONX₃, OX₇, and O-alkyl-X₇ wherein
X₇ is selected from the group consisting of H, alkyl, NO₂, NO, P(O)(OX₈)₂, PH(O)(OX₈), S(O)ₖN(alkyl)₂, S(O)ₖX₈, S(O)ₖOX₈, COOX₈, CONX₈, SO₃H, and COX₈, wherein
X₈ is selected from the group consisting of H, alkyl, carbocyclic ring, heterocyclic ring, aromatic ring, heteroaromatic ring, and —CX₉═CHX₁₀, wherein
each of X₉ and X₁₀ is independently H or alkyl,
wherein m is an integer from 0 to 7,
j is an integer from 0 to about 6, or
k is an integer from 0 to about 2; or
R1 is -Q₂-(CH₂)ₙ—Z;
Q₂ is optionally present and if present is selected from the group consisting of —CH₂—NH, —CH₂—O, —CH₂—S, —CH₂—SO₂ and —CH₂—OSO₂;
n is an integer from 0 to about 7;
wherein Z is selected from the group consisting of H, halogen, CF₃, CF₂H, N₃, NCS, CN, NO₂, NX₁X₂, OX₃, SX₃, OAc, OSO₂X₃, O-acyl, S-acyl, SO₂-alkyl, SO-alkyl, SC(CH₃)₂COOX₈, OC(CH₃)₂COOX₈, C(CH₃)₂COOX₈, Si(alkyl)₃, alkyl-CN, O-aroyl, O(CH₂)ⱼOX₃, O(CH₂)ⱼNX₁X₂, NH-acyl, NH-aroyl, CHO, C(halogen)₃, COOX₃, SO₃H, SO₂NX₁X₂, CONX₁X₂, NHC(O)O-alkyl, NHSO2-alkyl, alkoxy, alkyl, alcohol, alkylmercapto, alkylamino, di-alkylamino, alkylsulfinyl or alkylsulfonyl, CX₄X₅X₆, —CH═CHX₈, and —C≡CX₈;
each of X₁ and X₂ is independently H or alkyl, or
X₁ and X₂ together form part of a heterocyclic ring having about 4 to about 7 ring members and optionally one additional heteroatom selected from O, N and S, or
X₁ and X₂ together form part of an imide ring having about 5 to about 6 members,
X₃ is selected from the group consisting of H, alkyl, aryl, NO₂, (CH₂)ₘCN, hydroxyloweralkyl, and alkyl-NX₁X₂,
each of X₄, X₅, and X₆ is independently selected from the group consisting of H, alkyl, carbocyclic ring, hydroxyloweralkyl, alkyl-OH, halogen, CN, SNO, S(SO₂)alkyl, NX₁X₂, COOX₃, CONX₃, OX₇, and O-alkyl-X₇ wherein
X₇ is selected from the group consisting of H, alkyl, NO₂, NO, P(O)(OX₈)₂, PH(O)(OX₈), S(O)ₖN(alkyl)₂, S(O)ₖX₈, S(O)ₖOX₈, COOX₈, CONX₈, SO₃H, and COX₈, wherein
X₈ is selected from the group consisting of H, alkyl, carbocyclic ring, heterocyclic ring, aromatic ring, heteroaromatic ring, and —CX₉═CHX₁₀, wherein
X₉ and X₁₀ is independently H or alkyl,
wherein m is an integer from 0 to 7,
j is an integer from 0 to about 6,
k is an integer from 0 to about 2; or
R1 is —(CH₂)ₘ-Q₁-(CH₂)ₙ—Z;
Q₁ is selected from the group consisting of NH, O, S, CH═CH, C≡C, CO, SO₂ and OSO₂;
m is an integer from 1 to about 7;
n is an integer from 0 to about 7;
Z is selected from the group consisting of a bicyclic ring, a heterobicyclic ring, a tricyclic ring, a heterotricyclic ring, a polycyclic ring and a heteropolycyclic ring; or
R1 is —(CH₂)ₘ-Q₁-(CH₂)ₙ—Z;
Q₁ is selected from the group consisting of NH, O, S, CH═CH, C≡C, CO, SO₂ and OSO₂;
m is an integer from 1 to about 7;
n is an integer from 0 to about 7;
Z is selected from the group consisting of a carbocyclic ring having about 4 to about 7 ring members, a heterocyclic ring having about 4 to about 7 ring members, an aromatic ring having about 5 to about 7 ring members, a heteroaromatic ring having about 5 to about 7 ring members; a bicyclic ring, a heterobicyclic ring, a tricyclic ring, a heterotricyclic ring, a polycyclic ring, a heteropolycyclic ring or any above group substituted on at least one available ring atom by an alkyl group or any above group substituted on at least one available ring nitrogen atom by a benzyl group, a substituted benzyl group, an alkoxybenzyl group, a substituted alkoxybenzyl group, a benzhydryl group or a substituted benzhydryl group; and wherein the connecting point between the —(CH₂)ₙ— group and the Z group can be any available ring carbon atom or any available ring nitrogen atom; or
R1 is —(CH₂)ₘ-Q₁-(CH₂)ₙ—Z; Q₁ is selected from the group consisting of NH, O, S, CH═CH, C≡C, CO, SO₂ and OSO₂;
m is an integer from 1 to about 7;
n is an integer from 0 to about 7; and
Z is selected from the group consisting of a 5 member unsaturated ring having 0 to 4 independently selected heteroatoms as ring members, a substituted 5 member unsaturated ring having 0 to 4 independently selected heteroatoms as ring members, a 6 member aromatic ring having 0 to 5 independently selected heteroatoms as ring members or a substituted 6 member aromatic ring having 0 to 5 independently selected heteroatoms; and wherein the connecting point between the —(CH₂)ₙ— group and the Z group can be any available ring carbon atom or any available ring nitrogen atom; or
R1 is —(CH₂)ₘ-Q₁-(CH₂)ₙ—Z;
Q₁ is selected from the group consisting of NH, O, S, CH═CH, C≡C, CO, SO₂ and OSO₂;
m is an integer from 1 to about 7;
n is an integer from 0 to about 7;
Z is selected from the group consisting of 1-, 2- or 3-pyrrolidinyl, 1-, 2-, 3- or 4-piperidinyl, 2-, 3- or 4-morpholinyl, 2-, 3- or 4-thiomorpholinyl, 1-, 2- or 3-azetidinyl, 1- or 2-piperazinyl, 2- or 3-tetrahydrofuranyl; or any above group substituted on at least one available ring atom by an alkyl group; or any above group substituted on at least one available ring nitrogen atom by a benzyl group, a substituted benzyl group, an alkoxybenzyl group, a substituted alkoxybenzyl group, a benzhydryl group or a substituted benzhydryl group; and wherein the connecting point between the —(CH₂)ₙ— group and the Z group can be any available ring carbon atom or any available ring nitrogen atom; or
R1 is —(CH₂)ₘ-Q₁-(CH₂)ₙ—Z;
Q₁ is selected from the group consisting of NH, O, S, CH═CH, C≡C, CO, SO₂ and OSO₂;
m is an integer from 1 to about 7;
n is an integer from 0 to about 7; and
Z is selected from the group consisting of

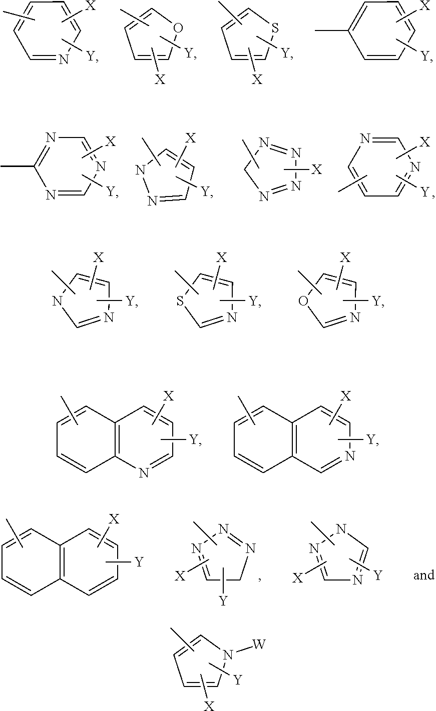

wherein each of X and Y is independently selected from the group consisting of H, halogen, CF₃, CF₂H, N₃, NCS, CN, NO₂, NX₁X₂, OX₃, SX₃, OAc, OSO₂X₃, O-acyl, S-acyl, SO₂-alkyl, SO-alkyl, SC(CH₃)₂COOX₈, OC(CH₃)₂COOX₈, C(CH₃)₂COOX₈, Si(alkyl)₃, alkyl-CN, O-aroyl, O(CH₂)ⱼOX₃, O(CH₂)ⱼNX₁X₂, NH-acyl, NH-aroyl, CHO, C(halogen)₃, COOX₃, SO₃H, SO₂NX₁X₂, CONX₁X₂, NHC(O)O-alkyl, NHSO2-alkyl, alkoxy, alkyl, alcohol, alkylmercapto, alkylamino, di-alkylamino, alkylsulfinyl or alkylsulfonyl, CX₄X₅X₆, —CH═CHX₈, and —C≡CX₈;
each of X₁ and X₂ is independently H or alkyl, or
X₁ and X₂ together form part of a heterocyclic ring having about 4 to about 7 ring members and optionally one additional heteroatom selected from O, N and S, or
X₁ and X₂ together form part of an imide ring having about 5 to about 6 members,
X₃ is selected from the group consisting of H, alkyl, aryl, NO₂, (CH₂)ₘCN, hydroxyloweralkyl, and alkyl-NX₁X₂,
X₄, X₅, and X₆ is independently selected from the group consisting of H, alkyl, carbocyclic ring, hydroxyloweralkyl, alkyl-OH, halogen, CN, SNO, S(SO₂)alkyl, NX₁X₂, COOX₃, CONX₃, OX₇, and O-alkyl-X₇, wherein
X₇ is selected from the group consisting of H, alkyl, NO₂, NO, P(O)(OX₈)₂, PH(O)(OX₈), S(O)ₖN(alkyl)₂, S(O)ₖX₈, S(O)ₖOX₈, COOX₈, CONX₈, SO₃H, and COX₈, wherein
X₈ is selected from the group consisting of H, alkyl, carbocyclic ring, heterocyclic ring, aromatic ring, heteroaromatic ring, and —CX₉═CHX₁₀, wherein
each of X₉ and X₁₀ is independently H or alkyl,
wherein m is an integer from 0 to 7,
j is an integer from 0 to about 6,
k is an integer from 0 to about 2,
W comprises H or alkyl; or
R1 is —(CH₂)ₘ-Q₁-(CH₂)ₙ—Z;
Q₁ is selected from the group consisting of NH, O, S, CH═CH, C≡C, CO, SO₂ and OSO₂;
m is an integer from 1 to about 7;
n is an integer from 0 to about 7; and
Z is selected from the group consisting of an unsaturated ring having 5 ring atoms and 0 to 2 independently selected heteroatoms as ring members fused to an unsaturated ring having 5 ring atoms and 0 to 4 independently selected heteroatoms as ring members, an unsaturated ring having 5 ring atoms and 0 to 2 independently selected heteroatoms as ring members fused to an unsaturated ring having 6 or 7 ring atoms and 0 to 4 independently selected heteroatoms as ring members or an unsaturated ring having 6 ring atoms and 0 to 4 independently selected heteroatoms as ring members fused to an unsaturated ring having 6 or 7 ring atoms and 0 to 4 independently selected heteroatoms as ring members; or
R1 is —(CH₂)ₘ-Q₁-(CH₂)ₙ—Z;
Q₁ is selected from the group consisting of NH, O, S, CH═CH, C≡C, CO, SO₂ and OSO₂;
m is an integer from 1 to about 7;
n is an integer from 0 to about 7; and
Z is selected from the group consisting of

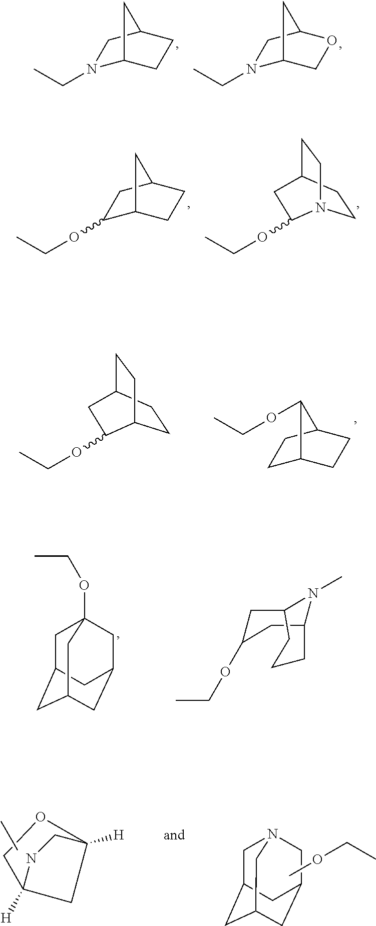

R1 is -T-(CH₂)ₙ—Z;
n is an integer from 0 to about 7;
T is selected from the group consisting of a carbocyclic ring having 3 to about 8 ring members, an unsaturated ring having 3 to about 8 carbon atoms as ring members, an aromatic ring having 5 to about 8 carbon atoms as ring members, a heterocyclic ring having 3 to about 8 ring members, a heteroaromatic ring having 5 to about 8 ring members, a bicyclic ring, a heterobicyclic ring, a tricyclic ring, a heterotricyclic ring, a polycyclic ring and a heteropolycyclic ring; and
Z is selected from the group consisting of, H, halogen, CF₃, CF₂H, N₃, NCS, CN, NO₂, NX₁X₂, OX₃, SX₃, OAc, OSO₂X₃, O-acyl, S-acyl, SO₂-alkyl, SO-alkyl, SC(CH₃)₂COOX₈, OC(CH₃)₂COOX₈, C(CH₃)₂COOX₈, Si(alkyl)₃, alkyl-CN, O-aroyl, O(CH₂)ⱼOX₃, O(CH₂)ⱼNX₁X₂, NH-acyl, NH-aroyl, CHO, C(halogen)₃, COOX₃, SO₃H, SO₂NX₁X₂, CONX₁X₂, NHC(O)O-alkyl, NHSO2-alkyl, alkoxy, alkyl, alcohol, alkylmercapto, alkylamino, di-alkylamino, alkylsulfinyl or alkylsulfonyl, CX₄X₅X₆, —CH═CHX₈, and —C≡CX₈;
each of X₁ and X₂ is independently H or alkyl, or
X₁ and X₂ together form part of a heterocyclic ring having about 4 to about 7 ring members and optionally one additional heteroatom selected from O, N and S, or
X₁ and X₂ together form part of an imide ring having about 5 to about 6 members,
X₃ is selected from the group consisting of H, alkyl, aryl, NO₂, (CH₂)ₘCN, hydroxyloweralkyl, and alkyl-NX₁X₂, each of X₄, X₅, and X₆ is independently selected from the group consisting of H, alkyl, carbocyclic ring, hydroxyloweralkyl, alkyl-OH, halogen, CN, SNO, S(SO₂)alkyl, NX₁X₂, COOX₃, CONX₃, OX₇, and O-alkyl-X₇, wherein
X₇ is selected from the group consisting of H, alkyl, NO₂, NO, P(O)(OX₈)₂, PH(O)(OX₈), S(O)ₖN(alkyl)₂, S(O)ₖX₈, S(O)ₖOX₈, COOX₈, CONX₈, SO₃H, and COX₈, wherein
X₈ is selected from the group consisting of H, alkyl, carbocyclic ring, heterocyclic ring, aromatic ring, heteroaromatic ring, and —CX₉═CHX₁₀, wherein
each of X₉ and X₁₀ is independently H or alkyl,
wherein m is an integer from 0 to 7,
j is an integer from 0 to about 6,
k is an integer from 0 to about 2; or
R1 is -T-(CH₂)ₙ—Z;
n is an integer from 0 to about 7;
T is selected from the group consisting of a carbocyclic ring having 3 to about 8 ring members, an unsaturated ring having 3 to about 8 carbon atoms as ring members, an aromatic ring having 5 to about 8 carbon atoms as ring members, a heterocyclic ring having 3 to about 8 ring members, a heteroaromatic ring having 5 to about 8 ring members, a bicyclic ring, a heterobicyclic ring, a tricyclic ring, a heterotricyclic ring, a polycyclic ring and a heteropolycyclic ring; and
Z is selected from the group consisting of a carbocyclic ring having about 4 to about 7 ring members, a heterocyclic ring having about 4 to about 7 ring members, an aromatic ring having about 5 to about 7 ring members, a heteroaromatic ring having about 5 to about 7 ring members, a bicyclic ring, a heterobicyclic ring, a tricyclic ring, a heterotricyclic ring, a polycyclic ring, a heteropolycyclic ring; or any above group substituted on at least one available ring atom by an alkyl group; or any above group substituted on at least one available ring nitrogen atom by a benzyl group, a substituted benzyl group, an alkoxybenzyl group, a substituted alkoxybenzyl group, a benzhydryl group or a substituted benzhydryl group; and wherein the connecting point between the —(CH₂)ₙ— group and the Z group can be any available ring carbon atom or any available ring nitrogen atom; or
R1 is -T-(CH₂)ₙ—Z;
n is an integer from 0 to about 7;
T is selected from the group consisting of a carbocyclic ring having 3 to about 8 ring members, an unsaturated ring having 3 to about 8 carbon atoms as ring members, an aromatic ring having 5 to about 8 carbon atoms as ring members, a heterocyclic ring having 3 to about 8 ring members, a heteroaromatic ring having 5 to about 8 ring members, a bicyclic ring, a heterobicyclic ring, a tricyclic ring, a heterotricyclic ring, a polycyclic ring and a heteropolycyclic ring; and
Z is selected from the group consisting of 1-, 2- or 3-pyrrolidinyl, 1-, 2-, 3- or 4-piperidinyl, 2-, 3- or 4-morpholinyl, 2-, 3- or 4-thiomorpholinyl, 1-, 2- or 3-azetidinyl, 1- or 2-piperazinyl, 2- or 3-tetrahydrofuranyl; or any above group substituted on at least one available ring atom by an alkyl group; or any above group substituted on at least one available ring nitrogen atom by a benzyl group, a substituted benzyl group, an alkoxybenzyl group, a substituted alkoxybenzyl group, a benzhydryl group or a substituted benzhydryl group; and wherein the connecting point between the —(CH₂)ₙ— group and the Z group can be any available ring carbon atom or any available ring nitrogen atom; or
R1 is -T-(CH₂)ₙ—Z;
n is an integer from 0 to about 7;
T is selected from the group consisting of a carbocyclic ring having 3 to about 8 ring members, an unsaturated ring having 3 to about 8 carbon atoms as ring members, an aromatic ring having 5 to about 8 carbon atoms as ring members, a heterocyclic ring having 3 to about 8 ring members, a heteroaromatic ring having 5 to about 8 ring members, a bicyclic ring, a heterobicyclic ring, a tricyclic ring, a heterotricyclic ring, a polycyclic ring and a heteropolycyclic ring; and
Z is selected from the group consisting of

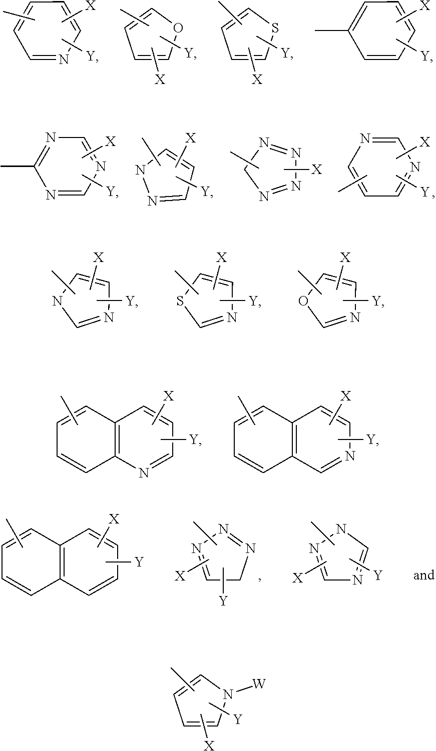

wherein X and Y are independently selected from the group consisting of H, acyl, SO₂-alkyl, SO-alkyl, SC(CH₃)₂COOX₈, OC(CH₃)₂COOX₈, C(CH₃)₂COOX₈, Si(alkyl)₃, alkyl-CN, O-aroyl, O(CH₂)ⱼOX₃, O(CH₂)ⱼNX₁X₂, NH-acyl, NH-aroyl, CHO, C(halogen)₃, COOX₃, SO₃H, SO₂NX₁X₂, CONX₁X₂, NHC(O)O-alkyl, NHSO2-alkyl, alkoxy, alkyl, alcohol, alkylmercapto, alkylamino, di-alkylamino, alkylsulfinyl or alkylsulfonyl, CX₄X₅X₆, —CH═CHX₈, and —C≡CX₈;
each of X₁ and X₂ is independently H or alkyl, or
X₁ and X₂ together form part of a heterocyclic ring having about 4 to about 7 ring members and optionally one additional heteroatom selected from O, N and S, or
X₁ and X₂ together form part of an imide ring having about 5 to about 6 members,
X₃ is selected from the group consisting of H, alkyl, aryl, NO₂, (CH₂)ₘCN, hydroxyloweralkyl, and alkyl-NX₁X₂,
each of X₄, X₅, and X₆ is independently selected from the group consisting of H, alkyl, carbocyclic ring, hydroxyloweralkyl, alkyl-OH, halogen, CN, SNO, S(SO₂)alkyl, NX₁X₂, COOX₃, CONX₃, OX₇, and O-alkyl-X₇, wherein
X₇ is selected from the group consisting of H, alkyl, NO₂, NO, P(O)(OX₈)₂, PH(O)(OX₈), S(O)ₖN(alkyl)₂, S(O)ₖX₈, S(O)ₖOX₈, COOX₈, CONX₈, SO₃H, and COX₈, wherein
X₈ is selected from the group consisting of H, alkyl, carbocyclic ring, heterocyclic ring, aromatic ring, heteroaromatic ring, and —CX₉═CHX₁₀, wherein
each of X₉ and X₁₀ is independently H or alkyl,
wherein m is an integer from 0 to 7,
j is an integer from 0 to about 6,
k is an integer from 0 to about 2
W is H or alkyl; or
R1 is -T-(CH₂)ₙ—Z;
n is an integer from 0 to about 7;
T is selected from the group consisting of a carbocyclic ring having 3 to about 8 ring members, an unsaturated ring having 3 to about 8 carbon atoms as ring members, an aromatic ring having 5 to about 8 carbon atoms as ring members, a heterocyclic ring having 3 to about 8 ring members, a heteroaromatic ring having 5 to about 8 ring members, a bicyclic ring, a heterobicyclic ring, a tricyclic ring, a heterotricyclic ring, a polycyclic ring and a heteropolycyclic ring; and
Z is selected from the group consisting of an unsaturated ring having 5 ring atoms and 0 to 2 independently selected heteroatoms as ring members fused to an unsaturated ring having 5 ring atoms and 0 to 4 independently selected heteroatoms as ring members, an unsaturated ring having 5 ring atoms and 0 to 2 independently selected heteroatoms as ring members fused to an unsaturated ring having 6 or 7 ring atoms and 0 to 4 independently selected heteroatoms as ring members or an unsaturated ring having 6 ring atoms and 0 to 3 independently selected heteroatoms as ring members fused to an unsaturated ring having 6 or 7 ring atoms and 0 to 4 independently selected heteroatoms as ring members; or
R1 is -T-Q-(CH₂)ₙ—Z,
each n is independently an integer from 0 to about 7;
T is selected from the group consisting of a carbocyclic ring having 3 to about 8 ring members, an unsaturated ring having 3 to about 8 carbon atoms as ring members, an aromatic ring having 5 to about 8 carbon atoms as ring members, a heterocyclic ring having 3 to about 8 ring members, a heteroaromatic ring having 5 to about 8 ring members, a bicyclic ring, a heterobicyclic ring, a tricyclic ring, a heterotricyclic ring, a polycyclic ring and a heteropolycyclic ring;
Q is CH═CH or C≡C;
Z is selected from the group consisting of H, halogen, CF₃, CF₂H, N₃, NCS, CN, NO₂, NX₁X₂, OX₃, SX₃, OAc, OSO₂X₃, O-acyl, S-acyl, SO₂-alkyl, SO-alkyl, SC(CH₃)₂COOX₈, OC(CH₃)₂COOX₈, C(CH₃)₂COOX₈, Si(alkyl)₃, alkyl-CN, O-aroyl, O(CH₂)ⱼOX₃, O(CH₂)ⱼNX₁X₂, NH-acyl, NH-aroyl, CHO, C(halogen)₃, COOX₃, SO₃H, SO₂NX₁X₂, CONX₁X₂, NHC(O)O-alkyl, NHSO2-alkyl, alkoxy, alkyl, alcohol, alkylmercapto, alkylamino, di-alkylamino, alkylsulfinyl or alkylsulfonyl, CX₄X₅X₆, —CH═CHX₈, and —C≡CX₈;
each of X₁ and X₂ is independently H or alkyl, or
X₁ and X₂ together form part of a heterocyclic ring having about 4 to about 7 ring members and optionally one additional heteroatom selected from O, N and S, or
X₁ and X₂ together form part of an imide ring having about 5 to about 6 members,
X₃ is selected from the group consisting of H, alkyl, NO₂, (CH₂)ₘCN, hydroxyloweralkyl, and alkyl-NX₁X₂,
each of X₄, X₅, and X₆ is independently selected from the group consisting of H, alkyl, carbocyclic ring, hydroxyloweralkyl, alkyl-OH, halogen, CN, SNO, S(SO₂)alkyl, NX₁X₂, COOX₃, CONX₃, OX₇, and O-alkyl-X₇, wherein
X₇ is selected from the group consisting of H, alkyl, NO₂, NO, P(O)(OX₈)₂, PH(O)(OX₈), S(O)ₖN(alkyl)₂, S(O)ₖX₈, S(O)ₖOX₈, COOX₈, CONX₈, SO₃H, and COX₈, wherein
X₈ is selected from the group consisting of H, alkyl, carbocyclic ring, heterocyclic ring, aromatic ring, heteroaromatic ring, and —CX₉═CHX₁₀, wherein
each of X₉ and X₁₀ is independently H or alkyl,
wherein m is an integer from 0 to 7,
j is an integer from 0 to about 6, or
k is an integer from 0 to about 2; or
R1 is -T-Q-(CH₂)ₙ—Z;
each n is independently an integer from 0 to about 7;
T is selected from the group consisting of a carbocyclic ring having 3 to about 8 ring members, an unsaturated ring having 3 to about 8 carbon atoms as ring members, an aromatic ring having 5 to about 8 carbon atoms as ring members, a heterocyclic ring having 3 to about 8 ring members, a heteroaromatic ring having 5 to about 8 ring members, a bicyclic ring, a heterobicyclic ring, a tricyclic ring, a heterotricyclic ring, a polycyclic ring and a heteropolycyclic ring;
Q is CH═CH or C≡C; and
Z is selected from the group consisting of a carbocyclic ring having about 4 to about 7 ring members, a heterocyclic ring having about 4 to about 7 ring members, an aromatic ring having about 5 to about 7 ring members, a heteroaromatic ring having about 5 to about 7 ring members, a bicyclic ring, a heterobicyclic ring, a tricyclic ring, a heterotricyclic ring, a polycyclic ring, a heteropolycyclic ring; or any above group substituted on at least one available ring atom by an alkyl group; or any above group substituted on at least one available ring nitrogen atom by a benzyl group, a substituted benzyl group, an alkoxybenzyl group, a substituted alkoxybenzyl group, a benzhydryl group or a substituted benzhydryl group; and wherein the connecting point between the —(CH₂)ₙ— group and the Z group can be any available ring carbon atom or any available ring nitrogen atom; or
R1 is -T-Q-(CH₂)ₙ—Z;
each n is independently an integer from 0 to about 7;
T is selected from the group consisting of a carbocyclic ring having 3 to about 8 ring members, an unsaturated ring having 3 to about 8 carbon atoms as ring members, an aromatic ring having 5 to about 8 carbon atoms as ring members, a heterocyclic ring having 3 to about 8 ring members, a heteroaromatic ring having 5 to about 8 ring members, a bicyclic ring, a heterobicyclic ring, a tricyclic ring, a heterotricyclic ring, a polycyclic ring and a heteropolycyclic ring;
Q is CH═CH or C≡C; and
Z is selected from the group consisting of 1-, 2- or 3-pyrrolidinyl, 1-, 2-, 3- or 4-piperidinyl, 2-, 3- or 4-morpholinyl, 2-, 3- or 4-thiomorpholinyl, 1-, 2- or 3-azetidinyl, 1- or 2-piperazinyl, 2- or 3-tetrahydrofuranyl; or any above group substituted on at least one available ring atom by an alkyl group; or any above group substituted on at least one available ring nitrogen atom by a benzyl group, a substituted benzyl group, an alkoxybenzyl group, a substituted alkoxybenzyl group, a benzhydryl group or a substituted benzhydryl group; and wherein the connecting point between the —(CH₂)ₙ— group and the Z group can be any available ring carbon atom or any available ring nitrogen atom; or
R1 is -T-Q-(CH₂)ₙ—Z;
each n is independently an integer from 0 to about 7;
T is selected from the group consisting of a carbocyclic ring having 3 to about 8 ring members, an unsaturated ring having 3 to about 8 carbon atoms as ring members, an aromatic ring having 5 to about 8 carbon atoms as ring members, a heterocyclic ring having 3 to about 8 ring members, a heteroaromatic ring having 5 to about 8 ring members, a bicyclic ring, a heterobicyclic ring, a tricyclic ring, a heterotricyclic ring, a polycyclic ring and a heteropolycyclic ring;
Q is CH═CH or C≡C; and
Z is selected from the group consisting of

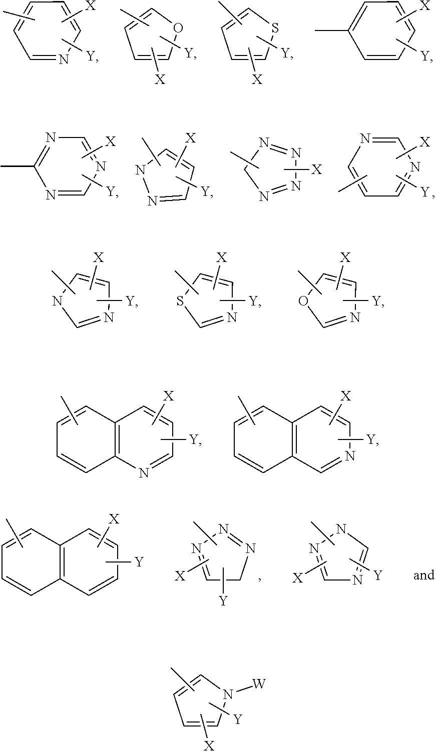

wherein X and Y are independently selected from the group consisting of H, halogen, CF₃, CF₂H, N₃, NCS, CN, NO₂, NX₁X₂, OX₃, SX₃, OAc, OSO₂X₃, O-acyl, S-acyl, SO₂-alkyl, SO-alkyl, SC(CH₃)₂COOX₈, OC(CH₃)₂COOX₈, C(CH₃)₂COOX₈, Si(alkyl)₃, alkyl-CN, O-aroyl, O(CH₂)ⱼOX₃, O(CH₂)ⱼNX₁X₂, NH-acyl, NH-aroyl, CHO, C(halogen)₃, COOX₃, SO₃H, SO₂NX₁X₂, CONX₁X₂, NHC(O)O-alkyl, NHSO2-alkyl, alkoxy, alkyl, alcohol, alkylmercapto, alkylamino, di-alkylamino, alkylsulfinyl or alkylsulfonyl, CX₄X₅X₆, —CH═CHX₈, and —C≡CX₈;
each of X₁ and X₂ is independently H or alkyl, or
X₁ and X₂ together form part of a heterocyclic ring having about 4 to about 7 ring members and optionally one additional heteroatom selected from O, N and S, or X₁ and X₂ together form part of an imide ring having about 5 to about 6 members,
X₃ is selected from the group consisting of H, alkyl, NO₂, (CH₂)ₘCN, hydroxyloweralkyl, and alkyl-NX₁X₂,
each of X₄, X₅, and X₆ is independently selected from the group consisting of H, alkyl, carbocyclic ring, hydroxyloweralkyl, alkyl-OH, halogen, CN, SNO, S(SO₂)alkyl, NX₁X₂, COOX₃, CONX₃, OX₇, and O-alkyl-X₇, wherein
X₇ is selected from the group consisting of H, alkyl, NO₂, NO, P(O)(OX₈)₂, PH(O)(OX₈), S(O)ₖN(alkyl)₂, S(O)ₖX₈, S(O)ₖOX₈, COOX₈, CONX₈, SO₃H, and COX₈, wherein
X₈ is selected from the group consisting of H, alkyl, carbocyclic ring, heterocyclic ring, aromatic ring, heteroaromatic ring, and —CX₉═CHX₁₀, wherein
each of X₉ and X₁₀ is independently H or alkyl,
wherein m is an integer from 0 to 7,
j is an integer from 0 to about 6,
k is an integer from 0 to about 2,
W is H or alkyl; or
R1 is -T-Q-(CH₂)ₙ—Z;
each n is independently an integer from 0 to about 7;
T is selected from the group consisting of a carbocyclic ring having 3 to about 8 ring members, an unsaturated ring having 3 to about 8 carbon atoms as ring members, an aromatic ring having 5 to about 8 carbon atoms as ring members, a heterocyclic ring having 3 to about 8 ring members, a heteroaromatic ring having 5 to about 8 ring members, a bicyclic ring, a heterobicyclic ring, a tricyclic ring, a heterotricyclic ring, a polycyclic ring and a heteropolycyclic ring;
Q is CH═CH or C≡C; and
Z is selected from the group consisting of an unsaturated ring having 5 ring atoms and 0 to 2 independently selected heteroatoms as ring members fused to an unsaturated ring having 5 ring atoms and 0 to 4 independently selected heteroatoms as ring members, an unsaturated ring having 5 ring atoms and 0 to 2 independently selected heteroatoms as ring members fused to an unsaturated ring having 6 or 7 ring atoms and 0 to 4 independently selected heteroatoms as ring members or an unsaturated ring having 6 ring atoms and 0 to 3 independently selected heteroatoms as ring members fused to an unsaturated ring having 6 or 7 ring atoms and 0 to 4 independently selected heteroatoms as ring members; or
R1 is -T-Q-(CH₂)ₙ—Z;
T is selected from the group consisting of a carbocyclic ring having 3 to about 8 ring members, an unsaturated ring having 3 to about 8 carbon atoms as ring members, an aromatic ring having 5 to about 8 carbon atoms as ring members, a heterocyclic ring having 3 to about 8 ring members, a heteroaromatic ring having 5 to about 8 ring members, a bicyclic ring, a heterobicyclic ring, a tricyclic ring, a heterotricyclic ring, a polycyclic ring and a heteropolycyclic ring;
each n is independently an integer from 0 to about 7;
Q is CH═CH or C≡C;
Z is

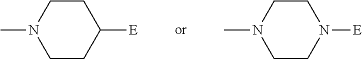

E is selected from the group consisting of a C1 to about C4, linear or branched alkyl group, a phenyl group, a substituted phenyl group, a benzyl group and a substituted benzyl group; or
R1 is -T-Q-(CH₂)ₙ—Z;
T is selected from the group consisting of a carbocyclic ring having 3 to about 8 ring members, an unsaturated ring having 3 to about 8 carbon atoms as ring members, an aromatic ring having 5 to about 8 carbon atoms as ring members, a heterocyclic ring having 3 to about 8 ring members, a heteroaromatic ring having 5 to about 8 ring members, a bicyclic ring, a heterobicyclic ring, a tricyclic ring, a heterotricyclic ring, a polycyclic ring and a heteropolycyclic ring;
each n is independently an integer from 0 to about 7;
Q is CH═CH or C≡C; and
Z is selected from the group consisting of

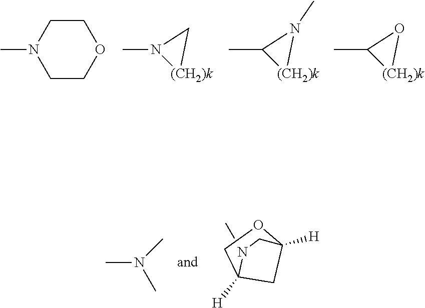

k is an integer from 1 to about 5, and
each of A₁ and A₂ is independently selected from the group consisting of a C1 to about C4 alkyl group, a phenyl group and a substituted phenyl group;
R2 is —(CH₂)ₙ—Z;
n is an integer from 0 to about 7; wherein
Z is selected from the group consisting of H, halogen, CF₃, CF₂H, N₃, NCS, CN, NO₂, NX₁X₂, OX₃, SX₃, OAc, OSO₂X₃, O-acyl, S-acyl, SO₂-alkyl, SO-alkyl, SC(CH₃)₂COOX₈, OC(CH₃)₂COOX₈, C(CH₃)₂COOX₈, Si(alkyl)₃, alkyl-CN, O-aroyl, O(CH₂)ⱼOX₃, O(CH₂)ⱼNX₁X₂, NH-acyl, NH-aroyl, CHO, C(halogen)₃, COOX₃, SO₃H, SO₂NX₁X₂, CONX₁X₂, NHC(O)O-alkyl, NHSO2-alkyl, alkoxy, alkyl, alcohol, alkylmercapto, alkylamino, di-alkylamino, alkylsulfinyl or alkylsulfonyl, CX₄X₅X₆, —CH═CHX₈, and —C≡CX₈;
each of X₁ and X₂ is independently H or alkyl, or
X₁ and X₂ together form part of a heterocyclic ring having about 4 to about 7 ring members and optionally one additional heteroatom selected from O, N and S, or
X₁ and X₂ together form part of an imide ring having about 5 to about 6 members,
X₃ is selected from the group consisting of H, alkyl, aryl, NO₂, (CH₂)ₘCN, hydroxyloweralkyl, and alkyl-NX₁X₂, each of X₄, X₅, and X₆ is independently selected from the group consisting of H, alkyl, carbocyclic ring, hydroxyloweralkyl, alkyl-OH, halogen, CN, SNO, S(SO₂)alkyl, NX₁X₂, COOX₃, CONX₃, OX₇, and O-alkyl-X₇, wherein
X₇ is selected from the group consisting of H, alkyl, NO₂, NO, P(O)(OX₈)₂, PH(O)(OX₈), S(O)ₖN(alkyl)₂, S(O)ₖX₈, S(O)ₖOX₈, COOX₈, CONX₈, SO₃H, and COX₈, wherein
X₈ is selected from the group consisting of H, alkyl, carbocyclic ring, heterocyclic ring, aromatic ring, heteroaromatic ring, and —CX₉═CHX₁₀, wherein
each of X₉ and X₁₀ is independently H or alkyl,
wherein m is an integer from 0 to 7,
j is an integer from 0 to about 6, or
k is an integer from 0 to about 2; or
R2 is —(CH₂)ₙ—Z;
n is an integer from 0 to about 7; and
Z is selected from the group consisting of a carbocyclic ring having about 4 to about 7 ring members, a heterocyclic ring having about 4 to about 7 ring members, an aromatic ring having about 5 to about 7 ring members, a heteroaromatic ring having about 5 to about 7 ring members, a bicyclic ring, a heterobicyclic ring, a tricyclic ring, a heterotricyclic ring, a polycyclic ring, a heteropolycyclic ring; or any above group substituted on at least one available ring atom by an alkyl group; or any above group substituted on at least one available ring nitrogen atom by a benzyl group, a substituted benzyl group, an alkoxybenzyl group, a substituted alkoxybenzyl group, a benzhydryl group or a substituted benzhydryl group; and wherein the connecting point between the —(CH₂)ₙ— group and the Z group can be any available ring carbon atom or any available ring nitrogen atom; or
R2 is —(CH₂)ₙ—Z;
n is an integer from 0 to about 7; and
Z is selected from the group consisting of a 5 member unsaturated ring having 0 to 4 independently selected heteroatoms as ring members, a substituted 5 member unsaturated ring having 0 to 4 independently selected heteroatoms as ring members, a 6 member aromatic ring having 0 to 5 independently selected heteroatoms as ring members or a substituted 6 member aromatic ring having 0 to 5 independently selected heteroatoms; and wherein the connecting point between the —(CH₂)ₙ— group and the Z group can be any available ring carbon atom or any available ring nitrogen atom; or
R2 is —(CH₂)ₙ—Z;
n is an integer from 0 to about 7; and
Z is selected from the group consisting of 1-, 2- or 3-pyrrolidinyl, 1-, 2-, 3- or 4-piperidinyl, 2-, 3- or 4-morpholinyl, 2-, 3- or 4-thiomorpholinyl, 1-, 2- or 3-azetidinyl, 1- or 2-piperazinyl, 2- or 3-tetrahydrofuranyl; or any above group substituted on at least one available ring atom by an alkyl group; or any above group substituted on at least one available ring nitrogen atom by a benzyl group, a substituted benzyl group, an alkoxybenzyl group, a substituted alkoxybenzyl group, a benzhydryl group or a substituted benzhydryl group; and wherein the connecting point between the —(CH₂)ₙ— group and the Z group can be any available ring carbon atom or any available ring nitrogen atom; or
R2 is —(CH₂)ₙ—Z;
n is an integer from 0 to about 7; and
Z is selected from the group consisting of

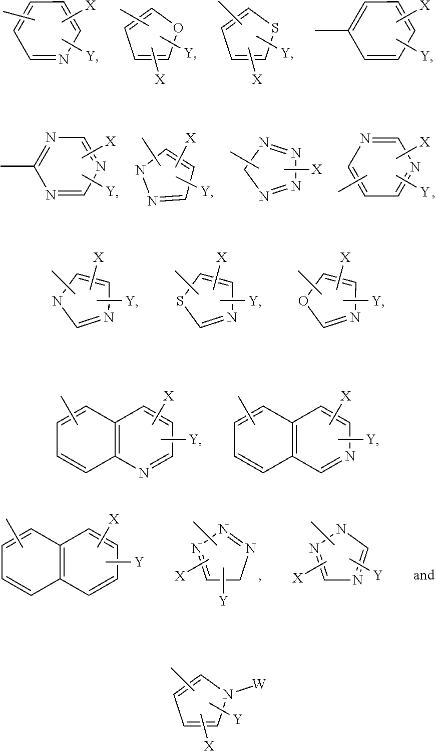

wherein each of X and Y is independently selected from the group consisting of H, acyl, SO₂-alkyl, SO-alkyl, SC(CH₃)₂COOX₈, OC(CH₃)₂COOX₈, C(CH₃)₂COOX₈, Si(alkyl)₃, alkyl-CN, O-aroyl, O(CH₂)ⱼOX₃, O(CH₂)ⱼNX₁X₂, NH-acyl, NH-aroyl, CHO, C(halogen)₃, COOX₃, SO₃H, SO₂NX₁X₂, CONX₁X₂, NHC(O)O-alkyl, NHSO2-alkyl, alkoxy, alkyl, alcohol, alkylmercapto, alkylamino, di-alkylamino, alkylsulfinyl or alkylsulfonyl, CX₄X₅X₆, —CH═CHX₈, and —C≡CX₈;
each of X₁ and X₂ is independently H or alkyl, or
X₁ and X₂ together form part of a heterocyclic ring having about 4 to about 7 ring members and optionally one additional heteroatom selected from O, N and S, or
X₁ and X₂ together form part of an imide ring having about 5 to about 6 members,
X₃ is selected from the group consisting of H, alkyl, aryl, NO₂, (CH₂)ₘCN, hydroxyloweralkyl, and alkyl-NX₁X₂, each of X₄, X₅, and X₆ is independently selected from the group consisting of H, alkyl, carbocyclic ring, hydroxyloweralkyl, alkyl-OH, halogen, CN, SNO, S(SO₂)alkyl, NX₁X₂, COOX₃, CONX₃, OX₇, and O-alkyl-X₇, wherein
X₇ is selected from the group consisting of H, alkyl, NO₂, NO, P(O)(OX₈)₂, PH(O)(OX₈), S(O)ₖN(alkyl)₂, S(O)ₖX₈, S(O)ₖOX₈, COOX₈, CONX₈, SO₃H, and COX₈, wherein
X₈ is selected from the group consisting of H, alkyl, carbocyclic ring, heterocyclic ring, aromatic ring, heteroaromatic ring, and —CX₉═CHX₁₀, wherein
each of X₉ and X₁₀ is independently H or alkyl,
wherein m is an integer from 0 to 7,
j is an integer from 0 to about 6, or
k is an integer from 0 to about 2,
W comprises H or alkyl; or
R2 is —(CH₂)ₙ—Z;
n is an integer from 0 to about 7; and
Z is selected from the group consisting of a carbocyclic ring having 6 ring atoms fused to a heterocyclic ring having from 5 to 7 ring atoms, a carbocyclic ring having 6 ring atoms fused to a heteroaromatic ring having from 5 to 7 ring atoms, a heterocyclic ring having 6 ring atoms fused to a heterocyclic ring having from 5 to 7 ring atoms, an heterocyclic ring having 6 ring atoms fused to a heteroaromatic ring having from 5 to 7 ring atoms, an aromatic ring having 6 ring atoms fused to a heterocyclic ring having from 5 to 7 ring atoms, an aromatic ring having 6 ring atoms fused to a heteroaromatic ring having from 5 to 7 ring atoms, a heteroaromatic ring having 6 ring atoms fused to a heterocyclic ring having from 5 to 7 ring atoms or a heteroaromatic ring having 6 ring atoms fused to a heteroaromatic ring having from 5 to 7 ring atoms; or
R2 is —(CH₂)ₙ—Z;
n is an integer from 0 to about 7; and
Z is selected from the group consisting of an unsaturated ring having 5 ring atoms and 0 to 2 independently selected heteroatoms as ring members fused to an unsaturated ring having 5 ring atoms and 0 to 4 independently selected heteroatoms as ring members, an unsaturated ring having 5 ring atoms and 0 to 2 independently selected heteroatoms as ring members fused to an unsaturated ring having 6 or 7 ring atoms and 0 to 3 independently selected heteroatoms as ring members or an unsaturated ring having 6 ring atoms and 0 to 4 independently selected heteroatoms as ring members fused to an unsaturated ring having 6 or 7 ring atoms and 0 to 4 independently selected heteroatoms as ring members; or
R2 is —(CH₂)ₘ-Q₁-(CH₂)ₙ—Z;
Q₁ is selected from the group consisting of NH, O, S, —CH═CH—, —C≡C—, —CO, SO₂ and OSO₂;
m is an integer from 1 to about 7;
n is an integer from 0 to about 7; and
wherein Z is selected from the group consisting of H, halogen, CF₃, CF₂H, N₃, NCS, CN, NO₂, NX₁X₂, OX₃, SX₃, OAc, OSO₂X₃, O-acyl, S-acyl, SO₂-alkyl, SO-alkyl, SC(CH₃)₂COOX₈, OC(CH₃)₂COOX₈, C(CH₃)₂COOX₈, Si(alkyl)₃, alkyl-CN, O-aroyl, O(CH₂)ⱼOX₃, O(CH₂)ⱼNX₁X₂, NH-acyl, NH-aroyl, CHO, C(halogen)₃, COOX₃, SO₃H, SO₂NX₁X₂, CONX₁X₂, NHC(O)O-alkyl, NHSO2-alkyl, alkoxy, alkyl, alcohol, alkylmercapto, alkylamino, di-alkylamino, alkylsulfinyl or alkylsulfonyl, CX₄X₅X₆, —CH═CHX₈, and —C≡CX₈;
each of X₁ and X₂ is independently H or alkyl, or
X₁ and X₂ together form part of a heterocyclic ring having about 4 to about 7 ring members and optionally one additional heteroatom selected from O, N and S, or
X₁ and X₂ together form part of an imide ring having about 5 to about 6 members,
X₃ is selected from the group consisting of H, alkyl, aryl, NO₂, (CH₂)ₘCN, hydroxyloweralkyl, and alkyl-NX₁X₂,
each of X₄, X₅, and X₆ is independently selected from the group consisting of H, alkyl, carbocyclic ring, hydroxyloweralkyl, alkyl-OH, halogen, CN, SNO, S(SO₂)alkyl, NX₁X₂, COOX₃, CONX₃, OX₇, and O-alkyl-X₇ wherein
X₇ is selected from the group consisting of H, alkyl, NO₂, NO, P(O)(OX₈)₂, PH(O)(OX₈), S(O)ₖN(alkyl)₂, S(O)ₖX₈, S(O)ₖOX₈, COOX₈, CONX₈, SO₃H, and COX₈, wherein
X₈ is selected from the group consisting of H, alkyl, carbocyclic ring, heterocyclic ring, aromatic ring, heteroaromatic ring, and —CX₉═CHX₁₀, wherein
each of X₉ and X₁₀ is independently H or alkyl,
wherein m is an integer from 0 to 7,
j is an integer from 0 to about 6,
k is an integer from 0 to about 2; or
R2 is -Q₂-(CH₂)ₙ—Z;
Q₂ is optionally present and if present is selected from the group consisting of —CH₂—NH, —CH₂—O, —CH₂—S, —CH₂—SO₂ and —CH₂—OSO₂;
n is an integer from 0 to about 7;
wherein Z is selected from the group consisting of H, halogen, CF₃, CF₂H, N₃, NCS, CN, NO₂, NX₁X₂, OX₃, SX₃, OAc, OSO₂X₃, O-acyl, S-acyl, SO₂-alkyl, SO-alkyl, SC(CH₃)₂COOX₈, OC(CH₃)₂COOX₈, C(CH₃)₂COOX₈, Si(alkyl)₃, alkyl-CN, O-aroyl, O(CH₂)ⱼOX₃, O(CH₂)ⱼNX₁X₂, NH-acyl, NH-aroyl, CHO, C(halogen)₃, COOX₃, SO₃H, SO₂NX₁X₂, CONX₁X₂, NHC(O)O-alkyl, NHSO2-alkyl, alkoxy, alkyl, alcohol, alkylmercapto, alkylamino, di-alkylamino, alkylsulfinyl or alkylsulfonyl, CX₄X₅X₆, —CH═CHX₈, and —C≡CX₈;
each of X₁ and X₂ is independently H or alkyl, or
X₁ and X₂ together form part of a heterocyclic ring having about 4 to about 7 ring members and optionally one additional heteroatom selected from O, N and S, or
X₁ and X₂ together form part of an imide ring having about 5 to about 6 members,
X₃ is selected from the group consisting of H, alkyl, aryl, NO₂, (CH₂)ₘCN, hydroxyloweralkyl, and alkyl-NX₁X₂,
each of X₄, X₅, and X₆ is independently selected from the group consisting of H, alkyl, carbocyclic ring, hydroxyloweralkyl, alkyl-OH, halogen, CN, SNO, S(SO₂)alkyl, NX₁X₂, COOX₃, CONX₃, OX₇, and O-alkyl-X₇, wherein
X₇ is selected from the group consisting of H, alkyl, NO₂, NO, P(O)(OX₈)₂, PH(O)(OX₈), S(O)ₖN(alkyl)₂, S(O)ₖX₈, S(O)ₖOX₈, COOX₈, CONX₈, SO₃H, and COX₈, wherein
X₈ is selected from the group consisting of H, alkyl, carbocyclic ring, heterocyclic ring, aromatic ring, heteroaromatic ring, and —CX₉═CHX₁₀, wherein
each of X₉ and X₁₀ is independently H or alkyl,
wherein m is an integer from 0 to 7,
j is an integer from 0 to about 6,
k is an integer from 0 to about 2; or
R2 is —(CH₂)ₘ-Q₁-(CH₂)ₙ—Z;
Q₁ is selected from the group consisting of NH, O, S, CH═CH, C≡C, CO, SO₂ and OSO₂;
m is an integer from 1 to about 7;
n is an integer from 0 to about 7;
Z is selected from the group consisting of a bicyclic ring, a heterobicyclic ring, a tricyclic ring, a heterotricyclic ring, a polycyclic ring and a heteropolycyclic ring; or
R2 is —(CH₂)ₘ-Q₁-(CH₂)ₙ—Z;
Q₁ is selected from the group consisting of NH, O, S, CH═CH, C≡C, CO, SO₂ and OSO₂;
m is an integer from 1 to about 7;
n is an integer from 0 to about 7;
Z is selected from the group consisting of a carbocyclic ring having about 4 to about 7 ring members, a heterocyclic ring having about 4 to about 7 ring members, an aromatic ring having about 5 to about 7 ring members, a heteroaromatic ring having about 5 to about 7 ring members; a bicyclic ring, a heterobicyclic ring, a tricyclic ring, a heterotricyclic ring, a polycyclic ring, a heteropolycyclic ring or any above group substituted on at least one available ring atom by an alkyl group or any above group substituted on at least one available ring nitrogen atom by a benzyl group, a substituted benzyl group, an alkoxybenzyl group, a substituted alkoxybenzyl group, a benzhydryl group or a substituted benzhydryl group; and wherein the connecting point between the —(CH₂)ₙ— group and the Z group can be any available ring carbon atom or any available ring nitrogen atom; or
R2 is —(CH₂)ₘ-Q₁-(CH₂)ₙ—Z;
Q₁ is selected from the group consisting of NH, O, S, CH═CH, C≡C, CO, SO₂ and OSO₂;
m is an integer from 1 to about 7;
n is an integer from 0 to about 7; and
Z is selected from the group consisting of a 5 member unsaturated ring having 0 to 4 independently selected heteroatoms as ring members, a substituted 5 member unsaturated ring having 0 to 4 independently selected heteroatoms as ring members, a 6 member aromatic ring having 0 to 5 independently selected heteroatoms as ring members or a substituted 6 member aromatic ring having 0 to 5 independently selected heteroatoms; and wherein the connecting point between the —(CH₂)ₙ— group and the Z group can be any available ring carbon atom or any available ring nitrogen atom; or
R2 is —(CH₂)ₘ-Q₁-(CH₂)ₙ—Z;
Q₁ is selected from the group consisting of NH, O, S, CH═CH, C≡C, CO, SO₂ and OSO₂;
m is an integer from 1 to about 7;
n is an integer from 0 to about 7;
Z is selected from the group consisting of 1-, 2- or 3-pyrrolidinyl, 1-, 2-, 3- or 4-piperidinyl, 2-, 3- or 4-morpholinyl, 2-, 3- or 4-thiomorpholinyl, 1-, 2- or 3-azetidinyl, 1- or 2-piperazinyl, 2- or 3-tetrahydrofuranyl; or any above group substituted on at least one available ring atom by an alkyl group; or any above group substituted on at least one available ring nitrogen atom by a benzyl group, a substituted benzyl group, an alkoxybenzyl group, a substituted alkoxybenzyl group, a benzhydryl group or a substituted benzhydryl group; and wherein the connecting point between the —(CH₂)ₙ— group and the Z group can be any available ring carbon atom or any available ring nitrogen atom; or
R2 is —(CH₂)ₘ-Q₁-(CH₂)ₙ—Z;
Q₁ is selected from the group consisting of NH, O, S, CH═CH, C≡C, CO, SO₂ and OSO₂;
m is an integer from 1 to about 7;
n is an integer from 0 to about 7; and
Z is selected from the group consisting of

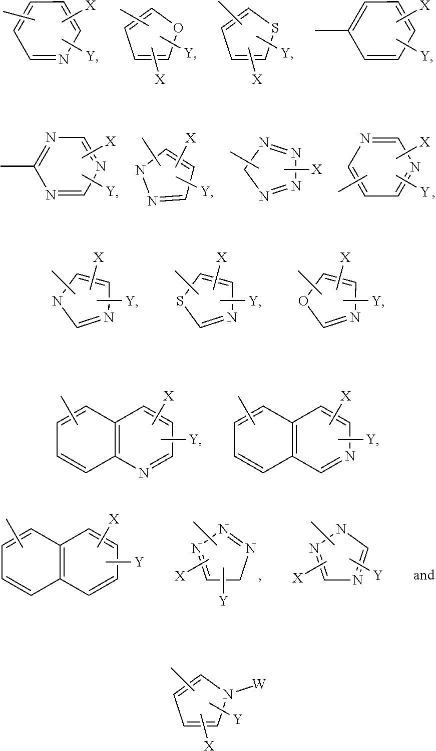

wherein each of X and Y is independently selected from the group consisting of H, acyl, SO₂-alkyl, SO-alkyl, SC(CH₃)₂COOX₈, OC(CH₃)₂COOX₈, C(CH₃)₂COOX₈, Si(alkyl)₃, alkyl-CN, O-aroyl, O(CH₂)ⱼOX₃, O(CH₂)ⱼNX₁X₂, NH-acyl, NH-aroyl, CHO, C(halogen)₃, COOX₃, SO₃H, SO₂NX₁X₂, CONX₁X₂, NHC(O)O-alkyl, NHSO2-alkyl, alkoxy, alkyl, alcohol, alkylmercapto, alkylamino, di-alkylamino, alkylsulfinyl or alkylsulfonyl, CX₄X₅X₆, —CH═CHX₈, and —C≡CX₈;
each of X₁ and X₂ is independently H or alkyl, or
X₁ and X₂ together form part of a heterocyclic ring having about 4 to about 7 ring members and optionally one additional heteroatom selected from O, N and S, or
X₁ and X₂ together form part of an imide ring having about 5 to about 6 members,
X₃ is selected from the group consisting of H, alkyl, aryl, NO₂, (CH₂)ₘCN, hydroxyloweralkyl, and alkyl-NX₁X₂, each of X₄, X₅, and X₆ is independently selected from the group consisting of H, alkyl, carbocyclic ring, hydroxyloweralkyl, alkyl-OH, halogen, CN, SNO, S(SO₂)alkyl, NX₁X₂, COOX₃, CONX₃, OX₇, and O-alkyl-X₇ wherein
X₇ is selected from the group consisting of H, alkyl, NO₂, NO, P(O)(OX₈)₂, PH(O)(OX₈), S(O)ₖN(alkyl)₂, S(O)ₖX₈, S(O)ₖOX₈, COOX₈, CONX₈, SO₃H, and COX₈, wherein
X₈ is selected from the group consisting of H, alkyl, carbocyclic ring, heterocyclic ring, aromatic ring, heteroaromatic ring, and —CX₉═CHX₁₀, wherein
each of X₉ and X₁₀ is independently H or alkyl,
wherein m is an integer from 0 to 7,
j is an integer from 0 to about 6,
k is an integer from 0 to about 2
W is H or alkyl; or
R2 is —(CH₂)ₘ-Q₁-(CH₂)ₙ—Z;
Q₁ is selected from the group consisting of NH, O, S, CH═CH, C≡C, CO, SO₂ and OSO₂;
m is an integer from 1 to about 7;
n is an integer from 0 to about 7; and
Z is selected from the group consisting of an unsaturated ring having 5 ring atoms and 0 to 2 independently selected heteroatoms as ring members fused to an unsaturated ring having 5 ring atoms and 0 to 4 independently selected heteroatoms as ring members, an unsaturated ring having 5 ring atoms and 0 to 2 independently selected heteroatoms as ring members fused to an unsaturated ring having 6 or 7 ring atoms and 0 to 4 independently selected heteroatoms as ring members or an unsaturated ring having 6 ring atoms and 0 to 4 independently selected heteroatoms as ring members fused to an unsaturated ring having 6 or 7 ring atoms and 0 to 4 independently selected heteroatoms as ring members; or
R2 is —(CH₂)ₘ-Q₁-(CH₂)ₙ—Z;
Q₁ is selected from the group consisting of NH, O, S, CH═CH, C≡C, CO, SO₂ and OSO₂;
m is an integer from 1 to about 7;
n is an integer from 0 to about 7; and
Z is selected from the group consisting of

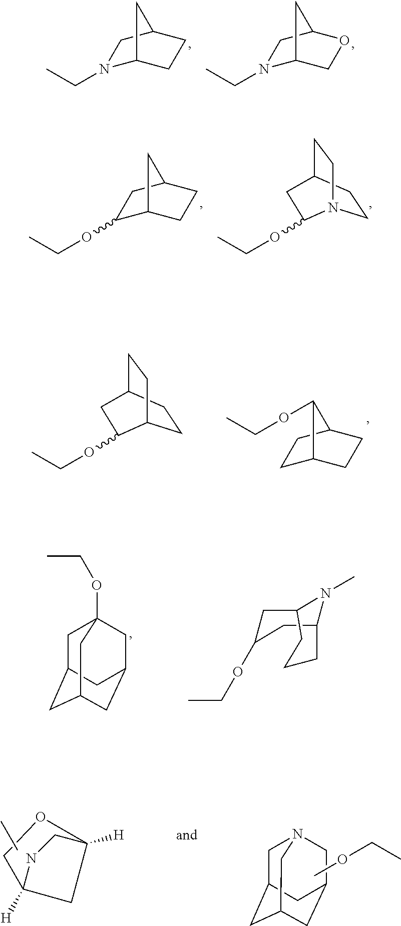

R2 is -T-(CH₂)ₙ—Z;
n is an integer from 0 to about 7;
T is selected from the group consisting of a carbocyclic ring having 3 to about 8 ring members, an unsaturated ring having 3 to about 8 carbon atoms as ring members, an aromatic ring having 5 to about 8 carbon atoms as ring members, a heterocyclic ring having 3 to about 8 ring members, a heteroaromatic ring having 5 to about 8 ring members, a bicyclic ring, a heterobicyclic ring, a tricyclic ring, a heterotricyclic ring, a polycyclic ring and a heteropolycyclic ring; and
Z is selected from the group consisting of, H, halogen, CF₃, CF₂H, N₃, NCS, CN, NO₂, NX₁X₂, OX₃, SX₃, OAc, OSO₂X₃, O-acyl, S-acyl, SO₂-alkyl, SO-alkyl, SC(CH₃)₂COOX₈, OC(CH₃)₂COOX₈, C(CH₃)₂COOX₈, Si(alkyl)₃, alkyl-CN, O-aroyl, O(CH₂)ⱼOX₃, O(CH₂)ⱼNX₁X₂, NH-acyl, NH-aroyl, CHO, C(halogen)₃, COOX₃, SO₃H, SO₂NX₁X₂, CONX₁X₂, NHC(O)O-alkyl, NHSO2-alkyl, alkoxy, alkyl, alcohol, alkylmercapto, alkylamino, di-alkylamino, alkylsulfinyl or alkylsulfonyl, CX₄X₅X₆, —CH═CHX₈, and —C≡CX₈;
each of X₁ and X₂ is independently H or alkyl, or
X₁ and X₂ together form part of a heterocyclic ring having about 4 to about 7 ring members and optionally one additional heteroatom selected from O, N and S, or
X₁ and X₂ together form part of an imide ring having about 5 to about 6 members,
X₃ is selected from the group consisting of H, alkyl, aryl, NO₂, (CH₂)ₘCN, hydroxyloweralkyl, or alkyl-NX₁X₂,
each of X₄, X₅, and X₆ is independently selected from the group consisting of H, alkyl, carbocyclic ring, hydroxyloweralkyl, alkyl-OH, halogen, CN, SNO, S(SO₂)alkyl, NX₁X₂, COOX₃, CONX₃, OX₇, and O-alkyl-X₇, wherein
X₇ is selected from the group consisting of H, alkyl, NO₂, NO, P(O)(OX₈)₂, PH(O)(OX₈), S(O)ₖN(alkyl)₂, S(O)ₖX₈, S(O)ₖOX₈, COOX₈, CONX₈, SO₃H, and COX₈, wherein
X₈ is selected from the group consisting of H, alkyl, carbocyclic ring, heterocyclic ring, aromatic ring, heteroaromatic ring, and —CX₉═CHX₁₀, wherein
each of X₉ and X₁₀ is independently H or alkyl, wherein
m is an integer from 0 to 7,
j is an integer from 0 to about 6,
k is an integer from 0 to about 2; or
R2 is -T-(CH₂)ₙ—Z;
n is an integer from 0 to about 7;
T is selected from the group consisting of a carbocyclic ring having 3 to about 8 ring members, an unsaturated ring having 3 to about 8 carbon atoms as ring members, an aromatic ring having 5 to about 8 carbon atoms as ring members, a heterocyclic ring having 3 to about 8 ring members, a heteroaromatic ring having 5 to about 8 ring members, a bicyclic ring, a heterobicyclic ring, a tricyclic ring, a heterotricyclic ring, a polycyclic ring and a heteropolycyclic ring; and
Z is selected from the group consisting of a carbocyclic ring having about 4 to about 7 ring members, a heterocyclic ring having about 4 to about 7 ring members, an aromatic ring having about 5 to about 7 ring members, a heteroaromatic ring having about 5 to about 7 ring members, a bicyclic ring, a heterobicyclic ring, a tricyclic ring, a heterotricyclic ring, a polycyclic ring, a heteropolycyclic ring; or any above group substituted on at least one available ring atom by an alkyl group; or any above group substituted on at least one available ring nitrogen atom by a benzyl group, a substituted benzyl group, an alkoxybenzyl group, a substituted alkoxybenzyl group, a benzhydryl group or a substituted benzhydryl group; and wherein the connecting point between the —(CH₂)ₙ— group and the Z group can be any available ring carbon atom or any available ring nitrogen atom; or
R2 is -T-(CH₂)ₙ—Z;
n is an integer from 0 to about 7;
T is selected from the group consisting of a carbocyclic ring having 3 to about 8 ring members, an unsaturated ring having 3 to about 8 carbon atoms as ring members, an aromatic ring having 5 to about 8 carbon atoms as ring members, a heterocyclic ring having 3 to about 8 ring members, a heteroaromatic ring having 5 to about 8 ring members, a bicyclic ring, a heterobicyclic ring, a tricyclic ring, a heterotricyclic ring, a polycyclic ring and a heteropolycyclic ring; and
Z is selected from the group consisting of 1-, 2- or 3-pyrrolidinyl, 1-, 2-, 3- or 4-piperidinyl, 2-, 3- or 4-morpholinyl, 2-, 3- or 4-thiomorpholinyl, 1-, 2- or 3-azetidinyl, 1- or 2-piperazinyl, 2- or 3-tetrahydrofuranyl; or any above group substituted on at least one available ring atom by an alkyl group; or any above group substituted on at least one available ring nitrogen atom by a benzyl group, a substituted benzyl group, an alkoxybenzyl group, a substituted alkoxybenzyl group, a benzhydryl group or a substituted benzhydryl group; and wherein the connecting point between the —(CH₂)ₙ— group and the Z group can be any available ring carbon atom or any available ring nitrogen atom; or
R2 is -T-(CH₂)ₙ—Z;
n is an integer from 0 to about 7;
T is selected from the group consisting of a carbocyclic ring having 3 to about 8 ring members, an unsaturated ring having 3 to about 8 carbon atoms as ring members, an aromatic ring having 5 to about 8 carbon atoms as ring members, a heterocyclic ring having 3 to about 8 ring members, a heteroaromatic ring having 5 to about 8 ring members, a bicyclic ring, a heterobicyclic ring, a tricyclic ring, a heterotricyclic ring, a polycyclic ring and a heteropolycyclic ring; and
Z is selected from the group consisting of

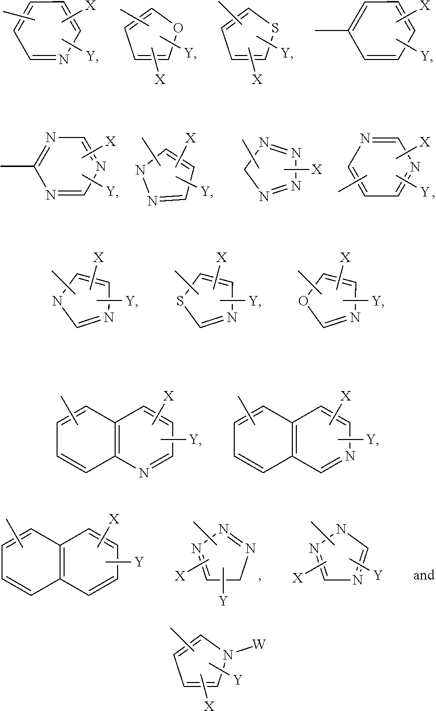

wherein each of X and Y is independently selected from the group consisting of H, halogen, CF₃, CF₂H, N₃, NCS, CN, NO₂, NX₁X₂, OX₃, SX₃, OAc, OSO₂X₃, O-acyl, S-acyl, SO₂-alkyl, SO-alkyl, SC(CH₃)₂COOX₈, OC(CH₃)₂COOX₈, C(CH₃)₂COOX₈, Si(alkyl)₃, alkyl-CN, O-aroyl, O(CH₂)ⱼOX₃, O(CH₂)ⱼNX₁X₂, NH-acyl, NH-aroyl, CHO, C(halogen)₃, COOX₃, SO₃H, SO₂NX₁X₂, CONX₁X₂, NHC(O)O-alkyl, NHSO2-alkyl, alkoxy, alkyl, alcohol, alkylmercapto, alkylamino, di-alkylamino, alkylsulfinyl or alkylsulfonyl, CX₄X₅X₆, —CH═CHX₈, and —C≡CX₈;
each of X₁ and X₂ is independently H or alkyl, or
X₁ and X₂ together form part of a heterocyclic ring having about 4 to about 7 ring members and optionally one additional heteroatom selected from O, N and S, or
X₁ and X₂ together form part of an imide ring having about 5 to about 6 members,
X₃ is selected from the group consisting of H, alkyl, aryl, NO₂, (CH₂)ₘCN, hydroxyloweralkyl, and alkyl-NX₁X₂,
each of X₄, X₅, and X₆ is independently selected from the group consisting of H, alkyl, carbocyclic ring, hydroxyloweralkyl, alkyl-OH, halogen, CN, SNO, S(SO₂)alkyl, NX₁X₂, COOX₃, CONX₃, OX₇, and O-alkyl-X₇, wherein
X₇ is selected from the group consisting of H, alkyl, NO₂, NO, P(O)(OX₈)₂, PH(O)(OX₈), S(O)ₖN(alkyl)₂, S(O)ₖX₈, S(O)ₖOX₈, COOX₈, CONX₈, SO₃H and COX₈, wherein
X₈ is selected from the group consisting of H, alkyl, carbocyclic ring, heterocyclic ring, aromatic ring, heteroaromatic ring, and —CX₉═CHX₁₀, wherein
each of X₉ and X₁₀ is independently H or alkyl, wherein
m is an integer from 0 to 7,
j is an integer from 0 to about 6,
k is an integer from 0 to about 2,
W comprises H or alkyl; or
R2 is -T-(CH₂)ₙ—Z;
n is an integer from 0 to about 7;
T is selected from the group consisting of a carbocyclic ring having 3 to about 8 ring members, an unsaturated ring having 3 to about 8 carbon atoms as ring members, an aromatic ring having 5 to about 8 carbon atoms as ring members, a heterocyclic ring having 3 to about 8 ring members, a heteroaromatic ring having 5 to about 8 ring members, a bicyclic ring, a heterobicyclic ring, a tricyclic ring, a heterotricyclic ring, a polycyclic ring and a heteropolycyclic ring; and
Z is selected from the group consisting of an unsaturated ring having 5 ring atoms and 0 to 2 independently selected heteroatoms as ring members fused to an unsaturated ring having 5 ring atoms and 0 to 4 independently selected heteroatoms as ring members, an unsaturated ring having 5 ring atoms and 0 to 2 independently selected heteroatoms as ring members fused to an unsaturated ring having 6 or 7 ring atoms and 0 to 4 independently selected heteroatoms as ring members or an unsaturated ring having 6 ring atoms and 0 to 3 independently selected heteroatoms as ring members fused to an unsaturated ring having 6 or 7 ring atoms and 0 to 4 independently selected heteroatoms as ring members; or
R2 is -T-(CH₂)ₘ-Q₁-(CH₂)ₙ—Z;
each of m and n is independently an integer from 0 to about 7;
T is selected from the group consisting of a carbocyclic ring having 3 to about 8 ring members, an unsaturated ring having 3 to about 8 carbon atoms as ring members, an aromatic ring having 5 to about 8 carbon atoms as ring members, a heterocyclic ring having 3 to about 8 ring members, a heteroaromatic ring having 5 to about 8 ring members, a bicyclic ring, a heterobicyclic ring, a tricyclic ring, a heterotricyclic ring, a polycyclic ring and a heteropolycyclic ring;
Q₁ is selected from the group consisting of NH, O, S, CH═CH, C≡C, CO, SO₂ and OSO₂; and
Z is selected from the group consisting of H, halogen, CF₃, CF₂H, N₃, NCS, CN, NO₂, NX₁X₂, OX₃, SX₃, OAc, OSO₂X₃, O-acyl, S-acyl, SO₂-alkyl, SO-alkyl, SC(CH₃)₂COOX₈, OC(CH₃)₂COOX₈, C(CH₃)₂COOX₈, Si(alkyl)₃, alkyl-CN, O-aroyl, O(CH₂)ⱼOX₃, O(CH₂)ⱼNX₁X₂, NH-acyl, NH-aroyl, CHO, C(halogen)₃, COOX₃, SO₃H, SO₂NX₁X₂, CONX₁X₂, NHC(O)O-alkyl, NHSO2-alkyl, alkoxy, alkyl, alcohol, alkylmercapto, alkylamino, di-alkylamino, alkylsulfinyl or alkylsulfonyl, CX₄X₅X₆, —CH═CHX₈, and —C≡CX₈;
each of X₁ and X₂ is independently H or alkyl, or
X₁ and X₂ together form part of a heterocyclic ring having about 4 to about 7 ring members and optionally one additional heteroatom selected from O, N and S, or
X₁ and X₂ together form part of an imide ring having about 5 to about 6 members,
X₃ is selected from the group consisting of H, alkyl, aryl, NO₂, (CH₂)ₘCN, hydroxyloweralkyl, and alkyl-NX₁X₂,
each of X₄, X₅, and X₆ is independently selected from the group consisting of H, alkyl, carbocyclic ring, hydroxyloweralkyl, alkyl-OH, halogen, CN, SNO, S(SO₂)alkyl, NX₁X₂, COOX₃, CONX₃, OX₇, and O-alkyl-X₇, wherein
X₇ comprises H, alkyl, NO₂, NO, P(O)(OX₈)₂, PH(O)(OX₈), S(O)ₖN(alkyl)₂, S(O)ₖX₈, S(O)ₖOX₈, COOX₈, CONX₈, SO₃H, and COX₈, wherein
X₈ is selected from the group consisting of a H, alkyl, carbocyclic ring, heterocyclic ring, aromatic ring, heteroaromatic ring, and —CX₉═CHX₁₀, wherein
each of X₉ and X₁₀ is independently H or alkyl, wherein
m is an integer from 0 to 7,
j is an integer from 0 to about 6,
k is an integer from 0 to about 2; or
R2 is -T-(CH₂)ₘ-Q₁-(CH₂)ₙ—Z;
each of m and n is independently an integer from 0 to about 7;
T is selected from the group consisting of a carbocyclic ring having 3 to about 8 ring members, an unsaturated ring having 3 to about 8 carbon atoms as ring members, an aromatic ring having 5 to about 8 carbon atoms as ring members, a heterocyclic ring having 3 to about 8 ring members, a heteroaromatic ring having 5 to about 8 ring members, a bicyclic ring, a heterobicyclic ring, a tricyclic ring, a heterotricyclic ring, a polycyclic ring and a heteropolycyclic ring;
Q₁ is selected from the group consisting of NH, O, S, CH═CH, C≡C, CO, SO₂ and OSO₂; and
Z is selected from the group consisting of a carbocyclic ring having about 4 to about 7 ring members, a heterocyclic ring having about 4 to about 7 ring members, an aromatic ring having about 5 to about 7 ring members, a heteroaromatic ring having about 5 to about 7 ring members, a bicyclic ring, a heterobicyclic ring, a polycyclic ring, a heteropolycyclic ring; or any above group substituted on at least one available ring atom by an alkyl group; or any above group substituted on at least one available ring nitrogen atom by a benzyl group, a substituted benzyl group, an alkoxybenzyl group, a substituted alkoxybenzyl group, a benzhydryl group or a substituted benzhydryl group; and wherein the connecting point between the —(CH₂)ₙ— group and the Z group can be any available ring carbon atom or any available ring nitrogen atom; or
R2 is -T-(CH₂)ₘ-Q₁-(CH₂)ₙ—Z;
each of m and n is independently an integer from 0 to about 7;
T is selected from the group consisting of a carbocyclic ring having 3 to about 8 ring members, an unsaturated ring having 3 to about 8 carbon atoms as ring members, an aromatic ring having 5 to about 8 carbon atoms as ring members, a heterocyclic ring having 3 to about 8 ring members, a heteroaromatic ring having 5 to about 8 ring members, a bicyclic ring, a heterobicyclic ring, a tricyclic ring, a heterotricyclic ring, a polycyclic ring and a heteropolycyclic ring;
Q₁ is selected from the group consisting of NH, O, S, CH═CH, C≡C, CO, SO₂ and OSO₂; and
Z is selected from the group consisting of 1-, 2- or 3-pyrrolidinyl, 1-, 2-, 3- or 4-piperidinyl, 2-, 3- or 4-morpholinyl, 2-, 3- or 4-thiomorpholinyl, 1-, 2- or 3-azetidinyl, 1- or 2-piperazinyl, 2- or 3-tetrahydrofuranyl; or any above group substituted on at least one available ring atom by an alkyl group; or any above group substituted on at least one available ring nitrogen atom by a benzyl group, a substituted benzyl group, an alkoxybenzyl group, a substituted alkoxybenzyl group, a benzhydryl group or a substituted benzhydryl group; and wherein the connecting point between the —(CH₂)ₙ— group and the Z group can be any available ring carbon atom or any available ring nitrogen atom; or
R2 is -T-(CH₂)ₘ-Q₁-(CH₂)ₙ—Z;
each of m and n is independently an integer from 0 to about 7;
T is selected from the group consisting of a carbocyclic ring having 3 to about 8 ring members, an unsaturated ring having 3 to about 8 carbon atoms as ring members, an aromatic ring having 5 to about 8 carbon atoms as ring members, a heterocyclic ring having 3 to about 8 ring members, a heteroaromatic ring having 5 to about 8 ring members, a bicyclic ring, a heterobicyclic ring, a tricyclic ring, a heterotricyclic ring, a polycyclic ring or a heteropolycyclic ring;
Q₁ is selected from the group consisting of N, O, S, CH═CH, C≡C, CO, SO₂ and OSO₂; and
Z is selected from the group consisting of

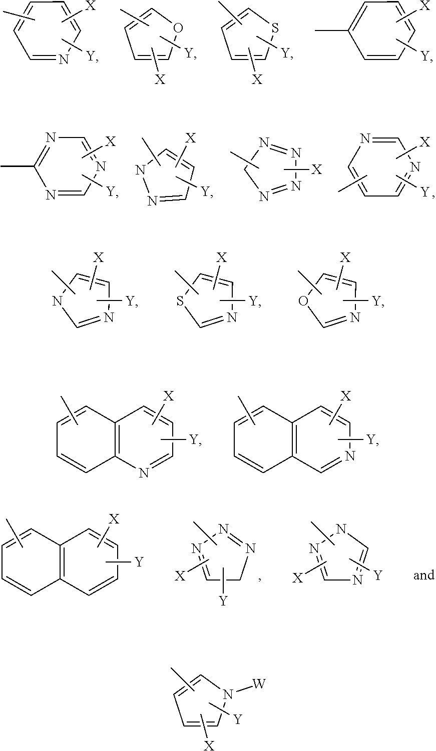

wherein each of X and Y is independently selected from the group consisting of H, halogen, CF₃, CF₂H, N₃, NCS, CN, NO₂, NX₁X₂, OX₃, SX₃, OAc, OSO₂X₃, O-acyl, S-acyl, SO₂-alkyl, SO-alkyl, SC(CH₃)₂COOX₈, OC(CH₃)₂COOX₈, C(CH₃)₂COOX₈, Si(alkyl)₃, alkyl-CN, O-aroyl, O(CH₂)ⱼOX₃, O(CH₂)ⱼNX₁X₂, NH-acyl, NH-aroyl, CHO, C(halogen)₃, COOX₃, SO₃H, SO₂NX₁X₂, CONX₁X₂, NHC(O)O-alkyl, NHSO2-alkyl, alkoxy, alkyl, alcohol, alkylmercapto, alkylamino, di-alkylamino, alkylsulfinyl or alkylsulfonyl, CX₄X₅X₆, —CH═CHX₈, and —C≡CX₈;
each of X₁ and X₂ is independently H or alkyl, or
X₁ and X₂ together form part of a heterocyclic ring having about 4 to about 7 ring members and optionally one additional heteroatom selected from O, N and S, or
X₁ and X₂ together form part of an imide ring having about 5 to about 6 members,
X₃ is selected from the group consisting of H, alkyl, aryl, NO₂, (CH₂)ₘCN, hydroxyloweralkyl, and alkyl-NX₁X₂,
each of X₄, X₅, and X₆ is independently selected from the group consisting of H, alkyl, carbocyclic ring, hydroxyloweralkyl, alkyl-OH, halogen, CN, SNO, S(SO₂)alkyl, NX₁X₂, COOX₃, CONX₃, OX₇, and O-alkyl-X₇, wherein
X₇ is selected from the group consisting of H, alkyl, NO₂, NO, P(O)(OX₈)₂, PH(O)(OX₈), S(O)ₖN(alkyl)₂, S(O)ₖX₈, S(O)ₖOX₈, COOX₈, CONX₈, SO₃H, and COX₈, wherein
X₈ is selected from the group consisting of H, alkyl, carbocyclic ring, heterocyclic ring, aromatic ring, heteroaromatic ring, and —CX₉═CHX₁₀, wherein
each of X₉ and X₁₀ is independently H or alkyl, wherein
m is an integer from 0 to 7,
j is an integer from 0 to about 6,
k is an integer from 0 to about 2,
W is H or alkyl; or
R2 is -T-(CH₂)ₘ-Q₁-(CH₂)ₙ—Z;
each of m and n is independently an integer from 0 to about 7;
T is selected from the group consisting of a carbocyclic ring having 3 to about 8 ring members, an unsaturated ring having 3 to about 8 carbon atoms as ring members, an aromatic ring having 5 to about 8 carbon atoms as ring members, a heterocyclic ring having 3 to about 8 ring members, a heteroaromatic ring having 5 to about 8 ring members, a bicyclic ring, a heterobicyclic ring, a tricyclic ring, a heterotricyclic ring, a polycyclic ring and a heteropolycyclic ring;
Q₁ is selected from the group consisting of NH, O, S, CH═CH, C≡C, CO, SO₂ and OSO₂; and
Z is selected from the group consisting of an unsaturated ring having 5 ring atoms and 0 to 2 independently selected heteroatoms as ring members fused to an unsaturated ring having 5 ring atoms and 0 to 2 independently selected heteroatoms as ring members, an unsaturated ring having 5 ring atoms and 0 to 2 independently selected heteroatoms as ring members fused to an unsaturated ring having 6 or 7 ring atoms and 0 to 4 independently selected heteroatoms as ring members or an unsaturated ring having 6 ring atoms and 0 to 3 independently selected heteroatoms as ring members fused to an unsaturated ring having 6 or 7 ring atoms and 0 to 4 independently selected heteroatoms as ring members, or
R2 is -T-(CH₂)ₘ-Q₁-(CH₂)ₙ—Z;
T is selected from the group consisting of a carbocyclic ring having 3 to about 8 ring members, an unsaturated ring having 3 to about 8 carbon atoms as ring members, an aromatic ring having 5 to about 8 carbon atoms as ring members, a heterocyclic ring having 3 to about 8 ring members, a heteroaromatic ring having 5 to about 8 ring members, a bicyclic ring, a heterobicyclic ring, a tricyclic ring, a heterotricyclic ring, a polycyclic ring and a heteropolycyclic ring;
each of m and n is independently an integer from 0 to about 7;
Q₁ is selected from the group consisting of NH, O, S, CH═CH, C≡C, CO, SO₂ and OSO₂; and
Z is

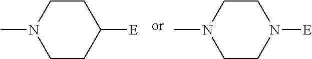

wherein E is selected from the group consisting of a C1 to about C4, linear or branched alkyl group, a phenyl group, a substituted phenyl group, a benzyl group and a substituted benzyl group; or
R2 is -T-(CH₂)ₘ-Q₁-(CH₂)ₙ—Z;
T is selected from the group consisting of a carbocyclic ring having 3 to about 8 ring members, an unsaturated ring having 3 to about 8 carbon atoms as ring members, an aromatic ring having 5 to about 8 carbon atoms as ring members, a heterocyclic ring having 3 to about 8 ring members, a heteroaromatic ring having 5 to about 8 ring members, a bicyclic ring, a heterobicyclic ring, a tricyclic ring, a heterotricyclic ring, a polycyclic ring and a heteropolycyclic ring;
each of m and n is independently an integer from 0 to about 7;
Q₁ is selected from the group consisting of NH, O, S, CH═CH, C≡C, CO, SO₂ and OSO₂;
Z is selected from the group consisting of

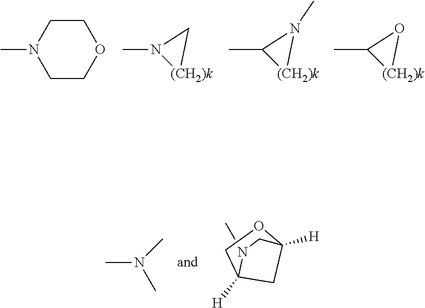

k is an integer from 1 to about 5,
each of A₁ and A₂ is independently selected from the group consisting of a C1 to about C4 alkyl group, a phenyl group and a substituted phenyl group;
R3 is

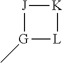

wherein G is selected from the group consisting of CH, C(CH₃), C(CN) and N,
each of L, K and J is independently selected from the group consisting of (CH₂)ₙ, (CH₃)₂, C═O, O, —CHOH, C(CH₃)OM₁, C(CH₂)ₙ(X)Y, NM₁, SO₂ SO and S,
and at least one of L, K or J is SO₂,
n is an integer from 0 to about 7;
M₁ is selected from the group consisting of H, alkyl, and C(O)M₂, wherein
M₂ is selected from the group consisting of H, alkyl, NM₃M₄, and OM₅, and each of M₃, M₄ and M₅ is independently H, OH or alkyl, and
each of X and Y is independently selected from the group consisting of H, halogen, CF₃, CF₂H, N₃, NCS, CN, NO₂, NX₁X₂, OX₃, SX₃, OAc, OSO₂X₃, O-acyl, S-acyl, SO₂-alkyl, SO-alkyl, SC(CH₃)₂COOX₈, OC(CH₃)₂COOX₈, C(CH₃)₂COOX₈, Si(alkyl)₃, alkyl-CN, O-aroyl, O(CH₂)ⱼOX₃, O(CH₂)ⱼNX₁X₂, NH-acyl, NH-aroyl, CHO, C(halogen)₃, COOX₃, SO₃H, SO₂NX₁X₂, CONX₁X₂, NHC(O)O-alkyl, NHSO2-alkyl, alkoxy, alkyl, alcohol, alkylmercapto, alkylamino, di-alkylamino, alkylsulfinyl or alkylsulfonyl, CX₄X₅X₆, —CH═CHX₈, and —C≡CX₈;
each of X₁ and X₂ is independently H or alkyl, or
X₁ and X₂ together form part of a heterocyclic ring having about 4 to about 7 ring members and optionally one additional heteroatom selected from O, N and S, or
X₁ and X₂ together form part of an imide ring having about 5 to about 6 members,
X₃ is selected from the group consisting of H, alkyl, NO₂, (CH₂)ₘCN, hydroxyloweralkyl, and alkyl-NX₁X₂,
each of X₄, X₅, and X₆ is independently selected from the group consisting of H, alkyl, carbocyclic ring, hydroxyloweralkyl, alkyl-OH, halogen, CN, SNO, S(SO₂)alkyl, NX₁X₂, COOX₃, CONX₃, OX₇, and O-alkyl-X₇, wherein
X₇ is selected from the group consisting of H, alkyl, NO₂, NO, P(O)(OX₈)₂, PH(O)(OX₈), S(O)ₖN(alkyl)₂, S(O)ₖX₈, S(O)ₖOX₈, COOX₈, CONX₈, SO₃H, and COX₈, wherein
X₈ is selected from the group consisting of H, alkyl, carbocyclic ring, heterocyclic ring, aromatic ring, heteroaromatic ring, and —CX₉═CHX₁₀, wherein
each of X₉ and X₁₀ is independently H or alkyl,
wherein m is an integer from 0 to 7,
j is an integer from 0 to about 6,
k is an integer from 0 to about 2; or
R4 is selected from the group consisting of H, halogen, CF₃, CF₂H, N₃, NCS, CN, NO₂, NX₁X₂, OH, ONO₂, SX₃, OAc, OSO₂X₃, O-acyl, S-acyl, SO₂-alkyl, SO-alkyl, SC(CH₃)₂COOX₈, OC(CH₃)₂COOX₈, C(CH₃)₂COOX₈, Si(alkyl)₃, alkyl-CN, O-aroyl, O(CH₂)ⱼOX₃, O(CH₂)ⱼNX₁X₂, NH-acyl, NH-aroyl, CHO, C(halogen)₃, COOX₃, SO₃H, SO₂NX₁X₂, CONX₁X₂, NHC(O)O-alkyl, NHSO2-alkyl, alkoxy, alkyl, alcohol, alkylmercapto, alkylamino, di-alkylamino, alkylsulfinyl or alkylsulfonyl, CX₄X₅X₆, —CH═CHX₈, and —C≡CX₈;
each of X₁ and X₂ is independently H or alkyl, or
X₁ and X₂ together form part of a heterocyclic ring having about 4 to about 7 ring members and optionally one additional heteroatom selected from O, N and S, or
X₁ and X₂ together form part of an imide ring having about 5 to about 6 members,
X₃ is selected from the group consisting of H, alkyl, NO₂, (CH₂)ₘCN, hydroxyloweralkyl, and alkyl-NX₁X₂,
each of X₄, X₅, and X₆ is independently selected from the group consisting of H, alkyl, carbocyclic ring, hydroxyloweralkyl, alkyl-OH, halogen, CN, SNO, S(SO₂)alkyl, NX₁X₂, COOX₃, CONX₃, OX₇, and O-alkyl-X₇, wherein
X₇ is selected from the group consisting of H, alkyl, NO₂, NO, P(O)(OX₈)₂, PH(O)(OX₈), S(O)ₖN(alkyl)₂, S(O)ₖX₈, S(O)ₖOX₈, COOX₈, CONX₈, SO₃H and COX₈, wherein
X₈ is selected from the group consisting of H, alkyl, carbocyclic ring, heterocyclic ring, aromatic ring, heteroaromatic ring, and —CX₉═CHX₁₀, wherein
each of X₉ and X₁₀ is independently H or alkyl, wherein
m is an integer from 0 to 7,
j is an integer from 0 to about 6,
k is an integer from 0 to about 2; or
R4 is selected from the group consisting of a carbocyclic ring having about 4 to about 7 ring members, a heterocyclic ring having about 4 to about 7 ring members, an aromatic ring having about 5 to about 7 ring members, a heteroaromatic ring having about 5 to about 7 ring members, a bicyclic ring, a heterobicyclic ring, a tricyclic ring, a heterotricyclic ring, a polycyclic ring and a heteropolycyclic ring; or
R4 is selected from the group consisting of

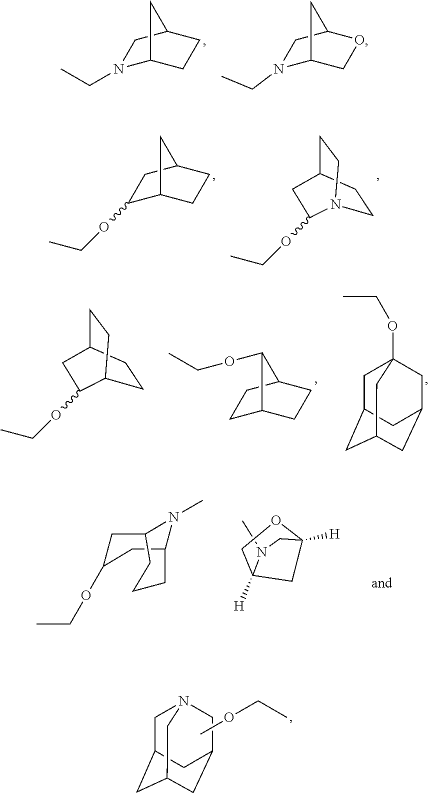

or
R4 is —(CH₂)_{d}—Z;
d is an integer from 1 to about 6;
Z is selected from the group consisting of H, halogen, CF₃, CF₂H, N₃, NCS, CN, NO₂, NX₁X₂, OX₃, SX₃, OAc, OSO₂X₃, O-acyl, S-acyl, SO₂-alkyl, SO-alkyl, SC(CH₃)₂COOX₈, OC(CH₃)₂COOX₈, C(CH₃)₂COOX₈, Si(alkyl)₃, alkyl-CN, O-aroyl, O(CH₂)ⱼOX₃, O(CH₂)ⱼNX₁X₂, NH-acyl, NH-aroyl, CHO, C(halogen)₃, COOX₃, SO₃H, SO₂NX₁X₂, CONX₁X₂, NHC(O)O-alkyl, NHSO2-alkyl, alkoxy, alkyl, alcohol, alkylmercapto, alkylamino, di-alkylamino, alkylsulfinyl or alkylsulfonyl, CX₄X₅X₆, —CH═CHX₈, and —C≡CX₈;
each of X₁ and X₂ is independently H or alkyl, or
X₁ and X₂ together form part of a heterocyclic ring having about 4 to about 7 ring members and optionally one additional heteroatom selected from O, N and S, or
X₁ and X₂ together form part of an imide ring having about 5 to about 6 members,
X₃ is selected from the group consisting of H, alkyl, NO₂, (CH₂)ₘCN, hydroxyloweralkyl, and alkyl-NX₁X₂,
each of X₄, X₅, and X₆ is independently selected from the group consisting of H, alkyl, carbocyclic ring, hydroxyloweralkyl, alkyl-OH, halogen, CN, SNO, S(SO₂)alkyl, NX₁X₂, COOX₃, CONX₃, OX₇, and O-alkyl-X₇, wherein
X₇ is selected from the group consisting of H, alkyl, NO₂, NO, P(O)(OX₈)₂, PH(O)(OX₈), S(O)ₖN(alkyl)₂, S(O)ₖX₈, S(O)ₖOX₈, COOX₈, CONX₈, SO₃H, and COX₈, wherein
X₈ is selected from the group consisting of H, alkyl, carbocyclic ring, heterocyclic ring, aromatic ring, heteroaromatic ring, and —CX₉═CHX₁₀, wherein
each of X₉ and X₁₀ is independently H or alkyl,
wherein m is an integer from 0 to 7,
j is an integer from 0 to about 6,
k is an integer from 0 to about 2; or
R4 is —(CH₂)_{d}—Z;
d is an integer from 1 to about 6; and
Z is selected from the group consisting of a carbocyclic ring having about 4 to about 7 ring members, a heterocyclic ring having about 4 to about 7 ring members, an aromatic ring having about 5 to about 7 ring members, a heteroaromatic ring having about 5 to about 7 ring members, a bicyclic ring, a heterobicyclic ring, a tricyclic ring, a heterotricyclic ring, a polycyclic ring, a heteropolycyclic ring; or any above group substituted on at least one available ring atom by an alkyl group; or any above group substituted on at least one available ring nitrogen atom by a benzyl group, a substituted benzyl group, an alkoxybenzyl group, a substituted alkoxybenzyl group, a benzhydryl group or a substituted benzhydryl group; and wherein the connecting point between the —(CH₂)_{d}— group and the Z group can be any available ring carbon atom or any available ring nitrogen atom; or
R4 is —(CH₂)_{d}—Z;
d is an integer from 1 to about 6; and
Z is selected from the group consisting of 1-, 2- or 3-pyrrolidinyl, 1-, 2-, 3- or 4-piperidinyl, 2-, 3- or 4-morpholinyl, 2-, 3- or 4-thiomorpholinyl, 1-, 2- or 3-azetidinyl, 1- or 2-piperazinyl, 2- or 3-tetrahydrofuranyl; or any above group substituted on at least one ring atom by an alkyl group; or any above group substituted on at least one available ring nitrogen atom by a benzyl group, a substituted benzyl group, an alkoxybenzyl group, a substituted alkoxybenzyl group, a benzhydryl group or a substituted benzhydryl group; and wherein the connecting point between the —(CH₂)_{d}— group and the Z group can be any available ring carbon atom or any available ring nitrogen atom; or
R4 is —(CH₂)ₘ-Q₁-(CH₂)ₙ—Z;
Q₁ is selected from the group consisting of NH, O, S, CH═CH, C≡C, CO, SO₂ and OSO₂;
m is an integer from 1 to about 7;
n is an integer from 0 to about 7; and
Z is selected from the group consisting of H, halogen, CF₃, CF₂H, N₃, NCS, CN, NO₂, NX₁X₂, OX₃, SX₃, OAc, OSO₂X₃, O-acyl, S-acyl, SO₂-alkyl, SO-alkyl, SC(CH₃)₂COOX₈, OC(CH₃)₂COOX₈, C(CH₃)₂COOX₈, Si(alkyl)₃, alkyl-CN, O-aroyl, O(CH₂)ⱼOX₃, O(CH₂)ⱼNX₁X₂, NH-acyl, NH-aroyl, CHO, C(halogen)₃, COOX₃, SO₃H, SO₂NX₁X₂, CONX₁X₂, NHC(O)O-alkyl, NHSO2-alkyl, alkoxy, alkyl, alcohol, alkylmercapto, alkylamino, di-alkylamino, alkylsulfinyl or alkylsulfonyl, CX₄X₅X₆, —CH═CHX₈, and —C≡CX₈;
each of X₁ and X₂ is independently H or alkyl, or
X₁ and X₂ together form part of a heterocyclic ring having about 4 to about 7 ring members and optionally one additional heteroatom selected from O, N and S, or
X₁ and X₂ together form part of an imide ring having about 5 to about 6 members,
X₃ is selected from the group consisting of H, alkyl, NO₂, (CH₂)ₘCN, hydroxyloweralkyl, and alkyl-NX₁X₂,
each of X₄, X₅, and X₆ is independently selected from the group consisting of H, alkyl, carbocyclic ring, hydroxyloweralkyl, alkyl-OH, halogen, CN, SNO, S(SO₂)alkyl, NX₁X₂, COOX₃, CONX₃, OX₇, and O-alkyl-X₇, wherein
X₇ is selected from the group consisting of H, alkyl, NO₂, NO, P(O)(OX₈)₂, PH(O)(OX₈), S(O)ₖN(alkyl)₂, S(O)ₖX₈, S(O)ₖOX₈, COOX₈, CONX₈, SO₃H, and COX₈, wherein
X₈ is selected from the group consisting of H, alkyl, carbocyclic ring, heterocyclic ring, aromatic ring, heteroaromatic ring, and —CX₉═CHX₁₀, wherein
each of X₉ and X₁₀ is independently H or alkyl,
wherein m is an integer from 0 to 7,
j is an integer from 0 to about 6, or
k is an integer from 0 to about 2; or
R4 is —(CH₂)ₘ-Q₁-(CH₂)ₙ—Z;
Q₁ is selected from the group consisting of NH, O, S, CH═CH, C≡C, CO, SO₂ and OSO₂;
m is an integer from 1 to about 7;
n is an integer from 0 to about 7; and
Z is selected from the group consisting of a carbocyclic ring having about 4 to about 7 ring members, a heterocyclic ring having about 4 to about 7 ring members, an aromatic ring having about 5 to about 7 ring members, a heteroaromatic ring having about 5 to about 7 ring members; a bicyclic ring, a heterobicyclic ring, a tricyclic ring, a heterotricyclic ring, a polycyclic ring, a heteropolycyclic ring; or any above group substituted on at least one available ring atom by an alkyl group; or any above group substituted on at least one available ring nitrogen atom by a benzyl group, a substituted benzyl group, an alkoxybenzyl group, a substituted alkoxybenzyl group, a benzhydryl group or a substituted benzhydryl group; and wherein the connecting point between the —(CH₂)ₙ— group and the Z group can be any available ring carbon atom or any available ring nitrogen atom; or
R4 is —(CH₂)ₘ-Q₁-(CH₂)ₙ—Z;
Q₁ is selected from the group consisting of NH, O, S, CH═CH, C≡C, CO, SO₂ and OSO₂;
m is an integer from 1 to about 7;
n is an integer from 0 to about 7; and
Z is selected from the group consisting of 1-, 2- or 3-pyrrolidinyl, 1-, 2-, 3- or 4-piperidinyl, 2-, 3- or 4-morpholinyl, 2-, 3- or 4-thiomorpholinyl, 1-, 2- or 3-azetidinyl, 1- or 2-piperazinyl, 2- or 3-tetrahydrofuranyl; or any above group substituted on at least one available ring atom by an alkyl; or any above group substituted on at least one available ring nitrogen atom by a benzyl group, a substituted benzyl group, an alkoxybenzyl group, a substituted alkoxybenzyl group, a benzhydryl group or a substituted benzhydryl group; and wherein the connecting point between the —(CH₂)ₙ— group and the Z group can be any available ring carbon atom or any available ring nitrogen atom; or
R4 is —(CH₂)ₘ-Q₁-(CH₂)ₙ—Z;
Q₁ is selected from the group consisting of NH, O, S, CH═CH, C≡C, CO, SO₂ and OSO₂;
m is an integer from 1 to about 7;
n is an integer from 0 to about 7; and
Z is selected from the group consisting of

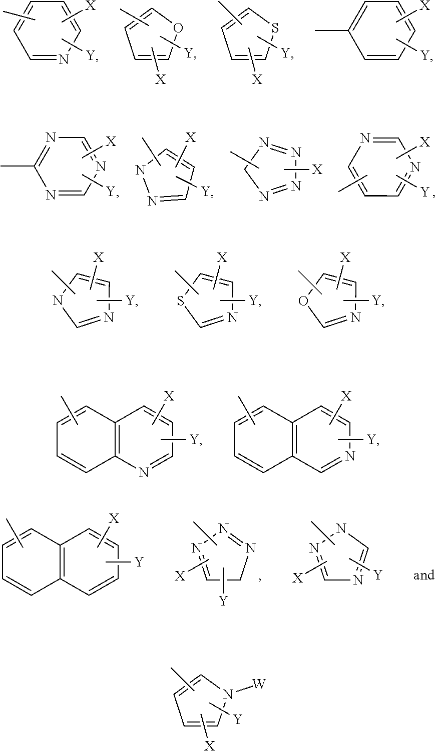

wherein each of X and Y is independently selected from the group consisting of H, halogen, CF₃, CF₂H, N₃, NCS, CN, NO₂, NX₁X₂, OX₃, SX₃, OAc, OSO₂X₃, O-acyl, S-acyl, SO₂-alkyl, SO-alkyl, SC(CH₃)₂COOX₈, OC(CH₃)₂COOX₈, C(CH₃)₂COOX₈, Si(alkyl)₃, alkyl-CN, O-aroyl, O(CH₂)ⱼOX₃, O(CH₂)ⱼNX₁X₂, NH-acyl, NH-aroyl, CHO, C(halogen)₃, COOX₃, SO₃H, SO₂NX₁X₂, CONX₁X₂, NHC(O)O-alkyl, NHSO2-alkyl, alkoxy, alkyl, alcohol, alkylmercapto, alkylamino, di-alkylamino, alkylsulfinyl or alkylsulfonyl, CX₄X₅X₆, —CH═CHX₈, and —C≡CX₈;
each of X₁ and X₂ is independently H or alkyl, or
X₁ and X₂ together form part of a heterocyclic ring having about 4 to about 7 ring members and optionally one additional heteroatom selected from O, N and S, or
X₁ and X₂ together form part of an imide ring having about 5 to about 6 members,
X₃ is selected from the group consisting of H, alkyl, NO₂, (CH₂)ₘCN, hydroxyloweralkyl, and alkyl-NX₁X₂,
each of X₄, X₅, and X₆ is independently selected from the group consisting of H, alkyl, carbocyclic ring, hydroxyloweralkyl, alkyl-OH, halogen, CN, SNO, S(SO₂)alkyl, NX₁X₂, COOX₃, CONX₃, OX₇, and O-alkyl-X₇, wherein
X₇ is selected from the group consisting of H, alkyl, NO₂, NO, P(O)(OX₈)₂, PH(O)(OX₈), S(O)ₖN(alkyl)₂, S(O)ₖX₈, S(O)ₖOX₈, COOX₈, CONX₈, SO₃H and COX₈, wherein
X₈ is selected from the group consisting of H, alkyl, carbocyclic ring, heterocyclic ring, aromatic ring, heteroaromatic ring, and —CX₉═CHX₁₀, wherein
each of X₉ and X₁₀ is independently H or alkyl,
m is an integer from 0 to 7,
j is an integer from 0 to about 6,
k is an integer from 0 to about 2, and
W is H or alkyl.

### DETAILED DESCRIPTION OF THE INVENTION

As used herein a “therapeutically effective amount” of a pharmaceutical composition or formulation in the present invention, is the quantity of a pharmaceutical composition or formulation which, when administered to an individual or animal, results in a sufficiently high level of that pharmaceutical composition or formulation in the individual or animal to cause a physiological response. The inventive pharmaceutical compositions or formulations described herein have pharmacological properties when administered in therapeutically effective amounts individually or in combination for providing a physiological response useful to treat fibrosis of the liver, lung or kidney, prostatic fibrosis and related inflammatory conditions including lower urinary tract symptoms, and benign prostatic hyperplasia.

Typically, a “therapeutically effective amount” of the inventive pharmaceutical composition or formulation is believed to range from about 0.01 mg/day to about 1,000 mg/day.

As used herein an “active pharmaceutical ingredient” is the main ingredient in the pharmaceutical composition or formulation disclosed in the invention that causes the desired effect of the medicine in an individual or an animal.

As used herein, an “individual” disclosed in the invention refers to a human. An “animal” disclosed in the invention refers to, for example nonhuman-primates such as squirrel monkeys, rhesus monkeys, marmosets, baboons, veterinary animals, such as rodents, dogs, cats, horses and the like, and farm animals, such as cows, pigs and the like.

In certain embodiments, the compound of Formula I or Formula II present in the pharmaceutical compositions or formulations disclosed in the invention could exist as pharmaceutically acceptable salts, solvates, hydrates, polymorphs, enantiomers, diastereomers, geometric isomers, racemates, tautomers, rotamers, atropisomers, isotopic variations, or N-oxides thereof,

Unless otherwise specifically defined, “acyl” refers to the general formula-C(O)alkyl.

Unless otherwise specifically defined, “acyloxy” refers to the general formula —O-acyl.

Unless otherwise specifically defined, “alcohol” refers to the general formula alkyl-OH and includes primary, secondary and tertiary variations.

Unless otherwise specifically defined, “alkyl” or “lower alkyl” refers to a linear, branched or cyclic alkyl group having from 1 to about 10 carbon atoms, and advantageously 1 to about 7 carbon atoms including, for example, methyl, ethyl, propyl, butyl, hexyl, octyl, isopropyl, isobutyl, tert-butyl, cyclopropyl, cyclohexyl, cyclooctyl, vinyl and allyl. The alkyl group can be saturated or unsaturated. The alkyl group can be unsubstituted, singly substituted or, if possible, multiply substituted, with substituent groups in any possible position. Unless otherwise specifically limited, a cyclic alkyl group includes monocyclic, bicyclic, tricyclic, tetracyclic and polycyclic rings, for example norbornyl, adamantyl and related terpenes.

Unless otherwise specifically defined, “alkoxy” refers to the general formula —O-alkyl.

Unless otherwise specifically defined, “alkylmercapto” refers to the general formula —S-alkyl.

Unless otherwise specifically defined, “alkylamino” refers to the general formula —(NH)-alkyl.

Unless otherwise specifically defined, “di-alkylamino” refers to the general formula —N-(alkyl)₂. Unless otherwise specifically limited di-alkylamino includes cyclic amine compounds such as piperidine and morpholine.

Unless otherwise specifically defined, an aromatic ring is an unsaturated ring structure having about 5 to about 7 ring members and including only carbon as ring atoms. The aromatic ring structure can be unsubstituted, singly substituted or, if possible, multiply substituted, with substituent groups in any possible position.

Unless otherwise specifically defined, “aryl” refers to an aromatic ring system that includes only carbon as ring atoms, for example phenyl, biphenyl or naphthyl. The aryl group can be unsubstituted, singly substituted or, if possible, multiply substituted, with substituent groups in any possible position.

Unless otherwise specifically defined, “aroyl” refers to the general formula —C(═O)-aryl.

Unless otherwise specifically defined, a bicyclic ring structure comprises 2 fused or bridged rings that include only carbon as ring atoms. The bicyclic ring structure can be saturated or unsaturated. The bicyclic ring structure can be unsubstituted, singly substituted or, if possible, multiply substituted, with substituent groups in any possible position. The individual rings may or may not be of the same type. Examples of bicyclic ring structures include naphthalene and bicyclooctane.

Unless otherwise specifically defined, a carbocyclic ring is a non-aromatic ring structure, saturated or unsaturated, having about 3 to about 8 ring members that includes only carbon as ring atoms, for example, cyclohexadiene or cyclohexane. The carbocyclic ring can be unsubstituted, singly substituted or, if possible, multiply substituted, with substituent groups in any possible position.

Unless otherwise specifically defined, “halogen” refers to an atom selected from fluorine, chlorine, bromine and iodine.

Unless otherwise specifically defined, a heteroaromatic ring is an unsaturated ring structure having about 5 to about 8 ring members independently selected from carbon atoms and one or more heteroatoms, including oxygen, nitrogen and/or sulfur, for example, pyridine, furan, quinoline, and their derivatives. The heteroaromatic ring can be unsubstituted, singly substituted or, if possible, multiply substituted, with substituent groups in any possible position.

Unless otherwise specifically defined, a heterobicyclic ring structure comprises 2 fused or bridged rings having ring members independently selected from carbon and one or more heteroatoms, including oxygen, nitrogen and/or sulfur. The heterobicyclic ring structure can be saturated or unsaturated. The heterobicyclic ring can be unsubstituted, singly substituted or, if possible, multiply substituted, with substituent groups in any possible position. The individual rings may or may not be of the same type. Examples of heterobicyclic ring structures include isobenzofuran and indole.

Unless otherwise specifically defined, a heterocyclic ring is a saturated or unsaturated ring structure having about 3 to about 8 ring members independently selected from carbon atoms and one or more heteroatoms, including oxygen, nitrogen and/or sulfur; for example, piperidine, morpholine, piperazine, pyrrolidine, thiomorpholine, 1,1-dioxothiomorpholine and their derivatives. The heterocyclic ring can be unsubstituted, singly substituted or, if possible, multiply substituted, with substituent groups in any possible position.

Unless otherwise specifically defined, a heterotricyclic ring structure comprises 3 fused, bridged, or both fused and bridged rings having ring members independently selected from carbon and one or more heteroatoms, including oxygen, nitrogen and/or sulfur. The heterotricyclic ring structure may be saturated or unsaturated. The heterotricyclic ring structure can be unsubstituted, singly substituted or, if possible, multiply substituted, with substituent groups in any possible position. The individual rings may or may not be of the same type. Examples of heterotricyclic ring structures include carbazole, phenanthroline, phenazine, 2,4,10-trioxaadamantane and tetradecahydro-phenanthroline.

Unless otherwise specifically defined, a heteropolycyclic ring structure comprises more than 3 rings that may be fused, bridged or both fused and bridged and that have ring members independently selected from carbon and one or more heteroatoms, including oxygen, nitrogen and/or sulfur. The heteropolycyclic ring structure can be saturated or unsaturated. The heteropolycyclic ring structure can be unsubstituted, singly substituted or, if possible, multiply substituted, with substituent groups in any possible position. The individual rings may or may not be of the same type. Examples of heteropolycyclic ring structures include azaadamantine, tropane, homotropane and 5-norbornene-2,3-dicarboximide.

Unless otherwise specifically defined, the term “phenacyl” refers to the general formula -phenyl-acyl.

Unless otherwise specifically defined, a polycyclic ring structure comprises more than 3 rings that may be fused, bridged or both fused and bridged, and that includes carbon as ring atoms. The polycyclic ring structure can be saturated or unsaturated. The polycyclic ring structure can be unsubstituted, singly substituted or, if possible, multiply substituted, with substituent groups in any possible position. The individual rings may or may not be of the same type. Examples of polycyclic ring structures include adamantine, bicyclooctane, norbornane and bicyclononanes.

Unless otherwise specifically defined, a spirocycle refers to a ring system wherein a single atom is the only common member of two rings. A spirocycle can comprise a saturated carbocyclic ring comprising about 3 to about 8 ring members, a heterocyclic ring comprising about 3 to about 8 ring atoms wherein up to about 3 ring atoms may be N, S, or O or a combination thereof.

Unless otherwise specifically defined, a tricyclic ring structure comprises 3 rings that may be fused, bridged or both fused and bridged, and that includes carbon as ring atoms. The tricyclic ring structure can be saturated or unsaturated. The tricyclic ring structure can be unsubstituted, singly substituted or, if possible, multiply substituted, with substituent groups in any possible position, and may be substituted or unsubstituted. The individual rings may or may not be of the same type. Examples of tricyclic ring structures include fluorene and anthracene.

Unless otherwise specifically limited the term substituted means substituted by a below-described substituent group in any possible position. Substituent groups for the above moieties useful in the invention are those groups that do not significantly diminish the biological activity of the inventive compound. Substituent groups that do not significantly diminish the biological activity of the inventive compound include, for example, H, halogen, N₃, NCS, CN, NO₂, NX₁X₂, OX₃, C(X₃)₃, OAc, O-acyl, O-aroyl, NH-acyl, NH-aroyl, NHCOalkyl, CHO, C(halogen)₃, COOX₃, SO₃H, PO₃H₂, SO₂NX₁X₂, CONX₁X₂, alkyl, alcohol, alkoxy, alkylmercapto, alkylamino, di-alkylamino, sulfonamide, thioalkoxy or methylene dioxy when the substituted structure has two adjacent carbon atoms, wherein X₁ and X₂ each independently comprise H or alkyl, or X₁ and X₂ together comprise part of a heterocyclic ring having about 4 to about 7 ring members and optionally one additional heteroatom selected from O, N or S, or X₁ and X₂ together comprise part of an imide ring having about 5 to about 6 members and X₃ comprises H, alkyl, hydroxyloweralkyl, or alkyl-NX₁X₂, wherein X₁-X₃ are as defined previously. Unless otherwise specifically limited a substituent group may be in any possible position.

In one embodiment, the polymer is a concentration enhancing polymer that increases bioavailability of the compound of claim 1, and is water soluble or dispersible in water.

In one embodiment, the polymer is selected from the group consisting of ionizable cellulosic polymers, non-ionizable cellulosic polymers, and vinyl polymers and copolymers having substituents selected from the group consisting of hydroxy, alkyl, acyloxy, and cyclic amide.

In one embodiment, the polymer is selected from the group consisting of hydroxypropyl methyl cellulose acetate succinate (HPMCAS), hydroxypropyl methyl cellulose phthalate (HPMCP), cellulose acetate phthalate (CAP), cellulose acetate trimellitate (CAT), methyl cellulose acetate phthalate, hydroxypropyl cellulose acetate phthalate, cellulose acetate terephthalate, cellulose acetate isophthalate, polyvinylpyrrolidinone, and polyvinylpyrrolidinone-polyvinylacetate copolymers.

In one embodiment, the polymer is an enteric polymer selected from the group consisting of cellulose derivatives including cellulose acetate phthalate (CAP), hydropropyl methylcellulose phthalate including HPMCP-50 or HPMCP-55, hydroxypropyl methylcellulose acetate succinate (HPMCAS), alkali-soluble acrylic copolymers inclusing Eudragit® L series and Eudragit® S series, polyvinyl acetate phthalate (PVAP), alginates, Carboxymethyl cellulose (CMC), and any combinations thereof.

In one embodiment, the polymer is a gastric-soluble polymer selected from the group consisting of methacrylic acid copolymers (such as Eudragit ER, Eudragit E100®), Eudragit E100 (also referred to as butylmethacylat-(2-dimethylaminoethyl)-methacrylat-methylmethacylat-copolymer (1:2:1), is a copolymer based on (2-dimethylaminoethyl) methacryalate, butyl methacrylate and methyl methacrylate having a mean molecular weight of about 150,000), chitosan and its derivatives (linear polysaccharide composed of randomly distributed-(l-4)-linked D-glucosamine (deacetylated unit) and N-acetyl-D-glucosamine (acetylated unit)), or other high molecular weight polymer with cationic function group, and any combinations thereof.

In one embodiment, the polymer is a pH-sensitive polymer selected from the group consisting of methacrylic acid copolymers including Eudragit E®, Eudragit E100®, Eudragit E100 (butylmethacylat-(2-dimethylaminoethyl)-methacrylat-methylmethacylat-copolymer (1:2:1), a copolymer based on (2-dimethyl-aminoethyl) methacryalate, butyl methacrylate and methyl methacrylate having a mean molecular weight of about 150,000), chitosan and its deritives (linear polysaccharide composed of randomly distributed-(l-4)-linked D-glucosamine (deacetylated unit) and N-acetyl-D-glucosamine (acetylated unit)), or other high molecule weigh polymer with cationic function group, cellulose derivatives such as cellulose acetate phthalate (CAP), hydropropyl methylcellulose phthalate including HPMCP-50 or HPMCP-55, hydroxypropyl methylcellulose acetate succinate (HPMCAS), alkali-soluble acrylic copolymers (Eudragit® L series and Eudragit® S series), polyvinyl acetate phthalate (PVAP), alginates, Carboxymethyl cellulose (CMC), and mixtures of one or more thereof.

In one embodiment, the polymer is a water soluble polymer selected from the group consisting homopolymers and copolymers of N-vinyl lactams, including polyvinylpyrrolidone (PVP), copolymers of N-vinyl pyrrolidone and vinyl acetate or vinyl propionate, polyvinyl caprolactam-polyvinyl acetate-polyethylene glycol graft copolymer, block copolymers of ethylene oxide and propylene oxide, lauroyl polyoxylglycerides cellulose esters and cellulose ethers including methylcellulose, hydroxyalkylcelluloses including hydroxypropylcellulose, hydroxyalkylalkylcelluloses including hydroxypropylmethylcellulose, high molecular poly alkylene oxides such as polyethylene oxide and polypropylene oxide and copolymers of ethylene oxide and propylene oxide, vinyl acetate polymers including copolymers of vinyl acetate and crotonic acid, partially saponified polyvinyl alcohol including partially hydrolyzed polyvinyl acetate, polyvinyl alcohol, oligo- and polysaccharides including carrageenans, galactomannans and xanthan gum, and mixtures thereof.

In one embodiment, the water-soluble polymer has a glass-transition temperature of at least 50° C.

In one embodiment, the polymer is an ionic or nonionic polymer selected from the group consisting from polymethylmethacrylates, polyvinylpyrrolidone, hydroxyethyl cellulose, hydroxypropyl cellulose, hydroxypropyl methylcellulose, ethylcellulose, polyvinylpyrrolidone-polyvinylalcohol, hydroxypropyl methylcellulose, acetate succinate (HPMC-AS), hydroxypropyl methylcellulose phthalate, cellulose acetate phthalate, hydroxypropyl cellulose acetate phthalate, methylcellulose acetate phthalate, polyoxyethylene-polyoxypropylene block copolymers, poloxamers, cyclodextrins, polyethylene glycol (PEG), polypropylene glycol (PPG), cellulose ethers and dextran.

In one embodiment, the polymer is selected from the group consisting of vinyl polymers and copolymers having substituents selected from the group consisting of hydroxy, alkyl, acyloxy, and cyclic amide.

In one embodiment, the polymer is selected from the group consisting of hypromellose phthalate and ethyl acrylate-methyl methacrylate-trimethylammonioethyl methacrylate chloride copolymer or hypromellose phthalate, and Eudragit.

In one embodiment, the polymer is selected from polyethylene glycol, polyvinylpyrrolidone, poly (2-ethyl-2-oxazaline), polyvinyl alcohol, hydroxypropyl cellulose and hydroxypropyl-methyl cellulose and alginate.

In one embodiment, the polymer is selected from the group consisting of polyvinylpyrrolidinone and polyvinylpyrrolidinone-polyvinylacetate copolymers.

In one embodiment, the polymer is a copovidone copolymer.

In one embodiment, the polymer is selected from HPMCP HP 55, HPMC E15, HPMC 100Cp, HPMC E4M, HPC Klucel, HPMC E5, Affinisol HPMCAS 716 (L), Affinisol HPMCAS 912 (M), Affinisol HPMCAS 126 (H), Kollidon VA64, PVP K30, Eudragit S100, Eudragit L100, Eudragit L100-55, Soluplus, ethyl cellulose, PEG 6000, PEG 1500, Poloxamer 188, Poloxamer 407, HPMCAS-L, HPMCAS-H, HPC-SSL, HPC-SL, Kollidon VA64, Kollidon K30, HPMCAS MMP, Gelucire 50/13, Compritol 888 ATO, Eudragit E PO, Ethocel Standard 10, Eudragit E PP, Eudragit E PO and combinations thereof.

In one embodiment, the surfactant or surfactant-like material is selected from the group consisting of anionic surfactants and nonionic surfactants.

In one embodiment, the surfactant or surfactant-like material is a homogeneous, amorphous or semi-crystalline powder.

In one embodiment, the surfactant or surfactant-like material is selected from the group consisting of polyoxyethylene alkyl ethers, polyoxyethylene alkylaryl ethers, polyethylene glycol fatty acid esters, alkylene glycol fatty acid mono esters, sucrose fatty acid esters, sorbitan fatty acid mono esters lauroyl polyoxylglycerides, polyvinyl caprolactam-polyvinyl acetate-polyethylene glycol graft copolymer marketed such as Soluplus®, sodium docusate, polyethylene glycol-26 glycerin marketed as Renex G26®, polyoxyehthylene monostearate, d-a-Tocopheryl polyethylene glycol 1000 succinate (vitamin E TPGS), polyoxyethylene alkyl ethers, polyethylene glycol fatty acid esters, alkylene glycol fatty acid mono esters, sucrose fatty acid esters, sorbitan fatty acid mono esters, sorbitan stearate, polyoxyethylene castor oil derivatives, or polyoxyethyleneglycerol oxystearate or block copolymers of ethylene oxide and propylene oxide, also known as polyoxyethylene polyoxypropylene block copolymers or polyoxyethylene polypropyleneglycol, such as Poloxamer or a mono fatty acid ester of polyoxyethylene sorbitan, e.g. polyoxyethylene sorbitan monooleate (Tween® 80), and mixtures thereof.

In one embodiment, the surfactant or surfactant-like material is non-ionic, is water soluble and is selected from sorbitan fatty acid esters, polyoxyethylene sorbitan fatty acid esters, polyoxyethylene castor oils, polyoxyethylene hydrogenated castor oils, vitamin E TPGS, Gelucires, and mixtures thereof.

In one embodiment, the surfactant or surfactant-like material is selected from the group consisting of fatty acid esters of saccharose, fatty alcohol ethers of oligoglucosides, fatty acid esters of glycerol, fatty acid esters of sorbitan, polyethoxylated fatty acid esters of sorbitan, fatty acid esters of poly(ethylene oxide), fatty alcohol ethers of poly(ethylene oxide), alkylphenol ethers of poly(ethylene oxide), polyoxyethylene-polyoxypropylene block copolymers, ethoxylated fats and oils.

In one embodiment, the surfactant or surfactant-like material is anionic.

In one embodiment, the surfactant or surfactant-like material is selected from polysorbates, poloxamines, carboxylates, alkyl sulfonates, alkylaryl sulfonates, alkyl sulfates, quaternary ammonium salts, phospholipids, medium-chain triglycerides, and long-chain triglycerides.

In one embodiment, the surfactant or surfactant-like material is present and is selected from Maisine® CC, Labrafac® Lipophile, castor oil, corn oil, Transcutol® HP, and PEG400.

In one embodiment, the surfactant or surfactant-like material is selected from the group consisting of polyethylene glycol-glyceryl triricinoleate (PEG-GTR), Labrasol® ALF, Gelucire® 44/14, Labrafil® M1944, Capryol® 90, Kolliphor RH40, Poloxamer P407, SLS, D-α-Tocopheryl polyethylene glycol 1000 succinate, and Tween 80.

In one embodiment, a carrier includes at least one of alkoxylated non-ionic surfactant, ether sulphate surfactant, cationic surfactant and ester surfactant.

In one embodiment, the excipient is selected from the group consisting of lactose monohydrate, microcrystalline cellulose, croscarmellose sodium, colloidal silicon dioxide, magnesium stearate, and combinations thereof.

In one embodiment, the excipient is selected from the group consisting polymer based excipients, surfactant based excipients and lipid based excipients.

In one embodiment, the excipient is a tonicity-adjusting agent listed by USP-NF selected from the group consisting of dextrose, glycerin, mannitol, potassium chloride and sodium chloride.

In one embodiment, the solvent is protic or aprotic.

In one embodiment, the a solvent is selected from acetone, ethyl acetate, methyl ethyl ketone, 2-propanol, water, acetone, ethanol, methanol, caustic water, chloroform, tetrahydrofuran, methylacetate, dichloromethane, and mixtures thereof.

By “physiologically acceptable salts” is meant, salts typically useful for pharmaceutical applications including acid addition salts and basic salts. Examples of acid addition salts are hydrochloride salts, hydrobromide salts, methane sulfonate salts etc. Examples of basic salts are salts where the cation is selected from alkali metals, such as sodium and potassium, alkaline earth metals, such as calcium, and ammonium ions. Other examples of physiologically acceptable salts can be found in “Remington's Pharmaceutical Sciences” 17. Ed. Alfonso R. Gennaro (Ed.), Mark Publishing Company, Easton, Pa., U.S.A., 1985 and more recent editions, and in Encyclopedia of Pharmaceutical Technology.

Polymorphic forms show improved physiochemical properties and stability. In one embodiment, the compounds present in the pharmaceutical compositions or formulations disclosed in the invention could exist in various solid forms. The solid forms can be crystalline and amorphous forms, but not limited to, solvates, hydrates, hydrolyzable esters and N-oxides of the compounds defined in the specification. These solid forms can be obtained by treating either the free base or their salts at a certain adjusted pH and certain temperature with an solvent or a combination of solvents. The solvents can be and not limited to a hydrocarbon solvent such as toluene, xylene, hexanes, heptane, or petroleum ether, alcohol such as methanol, ethanol, n-butanol, n-propanol and 2-propanol, di-isopropyl ether, ethyl-acetate, dichloromethane, acetic acid, acetone, tetrahydrofuran, dichloromethane, and water.

In certain embodiments, the application discloses a novel process of manufacturing pharmaceutical compositions or formulations of formula I-II.

In one embodiment, in order to improve the dehepaticability of the pharmaceutical compositions or formulations disclosed in the present invention for the required physiological effect, a “pro-drug” of the compound of Formula I or Formula II present in the pharmaceutical compositions or formulations can be made available. For example, the pro-drug can be a obtained by conjugation of the parent drug with polyethylene glycol(PEG).

In some embodiments, the compounds of Formula I or Formula II present in the pharmaceutical compositions or formulations disclosed in the invention have at least one fluorine atom incorporated at or adjacent to a site that is susceptible to metabolism. The fluorine atom can be part or adjacent to a substituent group as defined.

In another embodiment, the compounds of formula I-II present in the pharmaceutical compositions or formulations disclosed in the invention can also comprise isotopes at one or more of their atoms. For example, the compounds can be radiolabeled with isotopes, such as deuterium.

In some embodiments, the compounds of formula I-II present in the pharmaceutical compositions or formulations disclosed in the invention have at least one deuterium atom incorporated at or adjacent to a site that is susceptible to metabolism. The deuterium atom can be part or adjacent to a substituent group as defined.

In one embodiment, the compound of Formula I or Formula II present in the pharmaceutical compositions or formulations disclosed in the invention can be a “neutral antagonist”. These agents are said to have no effect on intrinsic receptor activity at least in certain test systems. However, these agents may be able to block receptor binding and activation, usually by a competitive agonist.

In some embodiments, the compound of Formula I or Formula II present in the pharmaceutical compositions or formulations disclosed in the invention exhibit essentially no CB1 receptor intrinsic activity and block or significantly reduce receptor activation by a suitable agonist. It would be further desirable to have pharmaceutical compositions or formulations disclosed in the invention that can be used to prevent, treat, or reduce the severity of symptoms of certain medical conditions. It would be especially desirable to have the pharmaceutical compositions or formulations that exhibit no or minimal side-effects in vivo.

In another embodiment, the compound of Formula I or Formula II present in the pharmaceutical compositions or formulations disclosed in the invention may act preferentially at the CB1 or CB2 receptors located in the periphery. In certain embodiments, the compounds do not penetrate the blood-brain-barrier, have restricted penetration or have slow penetration. In certain embodiments, peripherally acting compounds that are excluded from the CNS may have advantages over centrally acting compounds, for example, reduced psychotropic adverse effects.

In another embodiment, the compound of Formula I or Formula II present in the pharmaceutical compositions or formulations disclosed in the invention acts on the CB1 or CB2 receptors located in the periphery and could be behave either as a neutral antagonist, an inverse agonist or a partial antagonist.

In another embodiment, the compound of Formula I or Formula II present in the pharmaceutical compositions or formulations disclosed in the invention on the CB1 receptors located centrally, and could behave as a neutral antagonist, an inverse agonist or a partial antagonist.

In another embodiment, the compound of Formula I or Formula II present in the pharmaceutical compositions or formulations disclosed in the invention could act either as inverse agonists with no or reduced side effects.

In another embodiment, the compound of Formula I or Formula II present in the pharmaceutical compositions or formulations disclosed in the invention could act as partial agonists with no or reduced side effects.

The CB1 cannabinoid receptor binding affinities (Ki) for the compounds of Formula I or Formula II present in the pharmaceutical compositions or formulations disclosed in the invention range between 0.1 nM and less than 100 nM. For example, in one embodiment, human and rodent CB1 cannabinoid receptor binding affinities (Ki) are 1-5 nM and the human and rodent CB2 cannabinoid receptor binding affinity (Ki) are greater than 1000 nM. In another embodiment, human and rodent CB1 cannabinoid receptor binding affinities (Ki) are 1-5 nM and the human and rodent CB2 cannabinoid receptor binding affinity (Ki) are 1-5 nM, or human and rodent CB1 cannabinoid receptor binding affinities (Ki) are are greater than 1000 nM and the human and rodent CB2 cannabinoid receptor binding affinity (Ki) are 1-5 nM.

In one embodiment, the pharmaceutical compositions or formulations disclosed in the invention could in itself act as a drug with a combination effect. For example, pharmaceutical compositions or formulations disclosed in the invention could dually or exclusively act as a CB1 antagonist and a CB2 antagonist, CB2 agonist or 11ß-hydroxy steroid dehydrogenase-1 inhibitor. In certain embodiments, the compound could act dually as a CB1 antagonist, CB2 antagonist or CB2 agonist and a nitric oxide donor or a nitric oxide synthase inhibitor.

The pharmaceutical compositions or formulations of the present invention can be administered by a variety of known methods, including orally. The specific dosage level of active ingredient will depend upon a number of factors, including, for example, biological activity of the particular preparation, age, body weight, sex and general health of the individual being treated.

In another embodiment the disclosure also provides a pharmaceutical composition or formulation comprising a surface-active agent or an exogenous surfactant and a pharmaceutical composition or formulation disclosed in the invention to provide a pulmonary active drug. The surface-active agent has an affinity for the human alveolar/gas interface and comprises at least a portion of a mammalian pulmonary surfactant or a mimic thereof. In certain embodiments, this exogenous surfactant may act as a carrier of the pulmonary active drug and in some embodiments the carrier will facilitate the drug to reach the edematous areas of the lung while improving the bioavailability.

The disclosure also provides a method of treating a subject suffering from or at risk of suffering from a lung disease such as fibrosis, neonatal respiratory distress syndrome (RDS) and acute RDS (ARDS) that may occur due to COVID-19 comprising administering to the subject a mixture comprising a pharmaceutical composition or formulation of the present invention for lung treatment and a surface active agent in an amount effective to induce a drug effect in the lungs.

In certain embodiments, a compound of formula I-II present in the pharmaceutical compositions or formulations disclosed in the invention may be formulated with lavage-derived surfactants, minced lung surfactants or synthetic surfactants and may contain in various ratios a combination of dipalmitoyl phosphatidylcholine, unsaturated phosphatidylcholine, phosphatidylglycerol, other phospholipids including phosphatidylserine, phosphatidylethanolamine, phosphatidylinositol, lyso-phosphatidylcholine; and sphingomyelin, a neutral lipid such as Cholesterol and free fatty acids, in combination with surfactant proteins SP-B and SP—C. In some cases, synthetic additives such as Hexadecanol, Tyloxapol recombinant SP—C, KL4 peptide, SP—B and SP—C analogs that can mimic the native peptide sequence can be used instead.

As used herein, an “inflammatory condition” refers to a condition involving hepatic, renal, pulmonary disease or prostrate.

In a particular embodiment, the hepatic disease may be alcoholic or non-alcoholic steatohepatitis.

In a particular embodiment, the hepatic disease may be acute or chronic viral hepatitis including hepatitis C virus.

In a particular embodiment, the hepatic disease may be alcoholic or non-alcoholic cirrhosis.

In a particular embodiment, the hepatic disease may be liver cancer.

In a particular embodiment, the pulmonary disease may be idiopathic, genetic, radiation-induced or environmental agent induced, including chemicals.

In a particular embodiment, the renal disease may be chronic kidney disease including diabetic nephropathy

In a particular embodiment, the “inflammatory condition” can be relate to fibrosis of the lung, liver or kidney.

In a particular embodiment, fibrosis can be a condition related to the liver, lung, kidney or prostrate.

In a particular embodiment, the condition can be prostatic fibrosis and related inflammatory conditions including lower urinary tract symptoms.

In a particular embodiment, the inflammatory condition can be immune inflammatory disease such as benign prostatic hyperplasia.

Several embodiments of the invention are further described in the Specification. It will be recognized that one or more features of any embodiments disclosed herein may be combined and/or rearranged within the scope of the invention to produce further embodiments that are also within the scope of the invention.

Still other objects and advantages of the invention will become apparent by those of skill in the art from the disclosure herein, which are simply illustrative and not restrictive.

Thus, other embodiments will be recognized by the ordinarily skilled artisan without departing from the spirit and scope of the invention.

The disclosure is further illustrated by the following examples, which are not to be construed as limiting this disclosure in scope or spirit to the specific procedures described in this disclosure. It is to be understood that the examples are provided to illustrate certain embodiments and that no limitation to the scope of the disclosure is intended thereby. It is to be further understood that resort may be had to various other embodiments, modifications, and equivalents thereof which may suggest themselves to those skilled in the art without departing from the spirit of the present disclosure and/or scope of the appended claims.

The inventive embodiments are disclosed herein with reference to the following Examples and related description.

### Example 1

Micro-evaporation, X-ray diffraction (XRD) patterns and differential scanning calorimetry (DSC) were used to understand and determine amorphous and crystalline dispersion formulations for the API and for feasibility towards Spray Dried Dispersions (SDD) and/or extrudates (Hot melt Extrusion). Micro-evaporation studies were performed to identify the polymer and polymer/surfactant matrix that best enhanced solubility. Micro-evaporative dispersion method allowed for the parallel evaluation of multiple approaches, including different carriers, excipients, and solvents in various proportions, and concentrations. The results from micro-evaporation studies were leveraged into downstream development of the most suitable formulation to put on a spray dryer. API-polymer mixtures were prepared in an organic solvent and micro evaporation was performed in a speedvac concentrator. The dried films were re-suspended in pH 6.8 phosphate buffer by vortexing and transferring test samples to 96-well plates followed by dissolution analysis at pH 6.8 by UV absorbance. Re-dissolution behavior was measured to determine performance of matrix system. Concentrations were adjusted to 100-250 μg/mL for drug loads ranging between 10-25% for API formulations with either polymer alone or polymer/surfactant. Spray drying of the selected formulation was performed on a Buchi-B290 spray dryer. The solid content of the solution was fixed at an optimal wt % in an organic solvent of choice.

For example, using a formulation of Compound 1:HPMCAS L:HPC SSL (20:50:30), the solid content of the solution was fixed at 5 wt % in DCM:Methanol (80:20 by volume) with total solids of 3 grams. The initial solubility assessment showed ˜100 mg/mL solubility of API in the solvent mixture. The spray drying conditions are shown below:

| | Inlet | Outlet | | | | |
|---|---|---|---|---|---|---|
| | Temp. | Temp. | Aspirator | Gas flow | Feed rate | % |
| Variable | (° C.) | (° C.) | (%) | (L/hr.) | (g/min) | Yield |
| Value | 75 | 53-57 | 95 | 470 | 6.5 | 66.7 |

The powder sample was collected from the collection vessel and cyclone with a final yield of 2 grams (66%). pXRD analysis of the new formulation confirmed amorphous nature of the SDD. In another example of Compound 1:HPMCAS L:TPGS (20:70:10), the outlet temperature was maintained at 50-51° C. with the other parameters remaining the same. Spray dried dispersions were analyzed for residual crystallinity using Rigaku Miniflex II X-ray diffractometer.

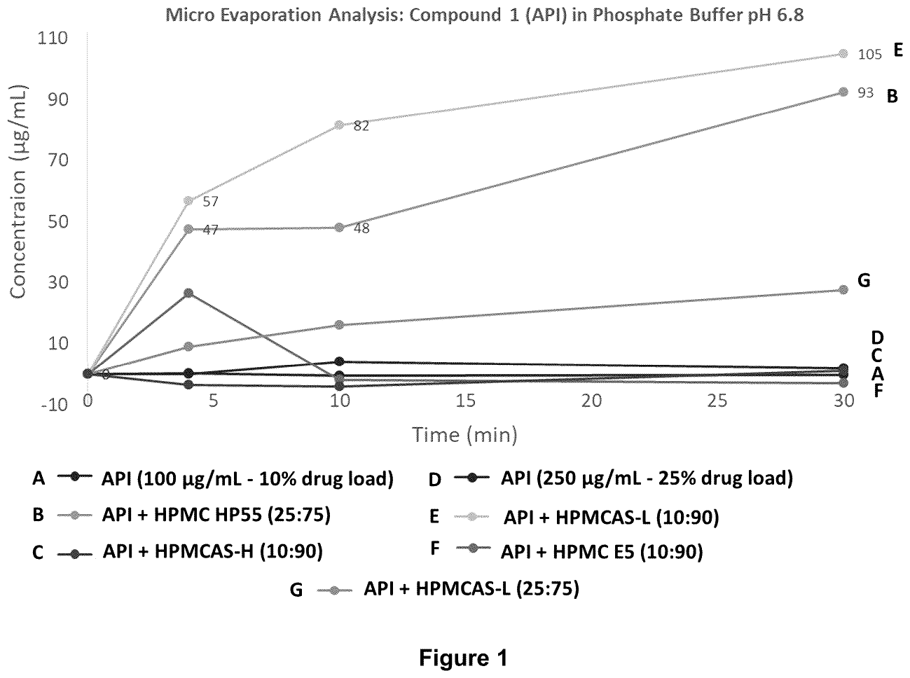

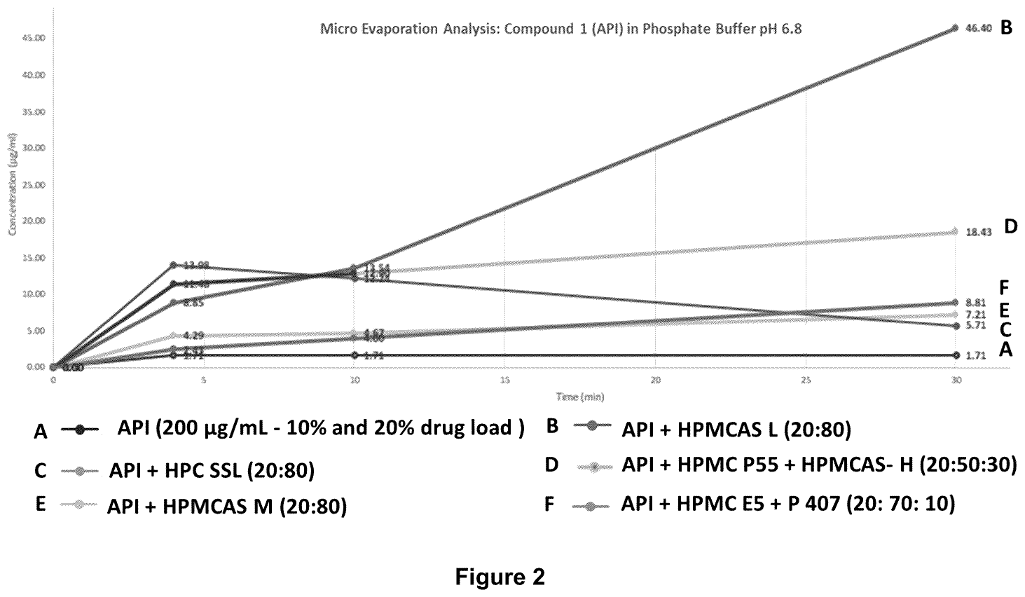

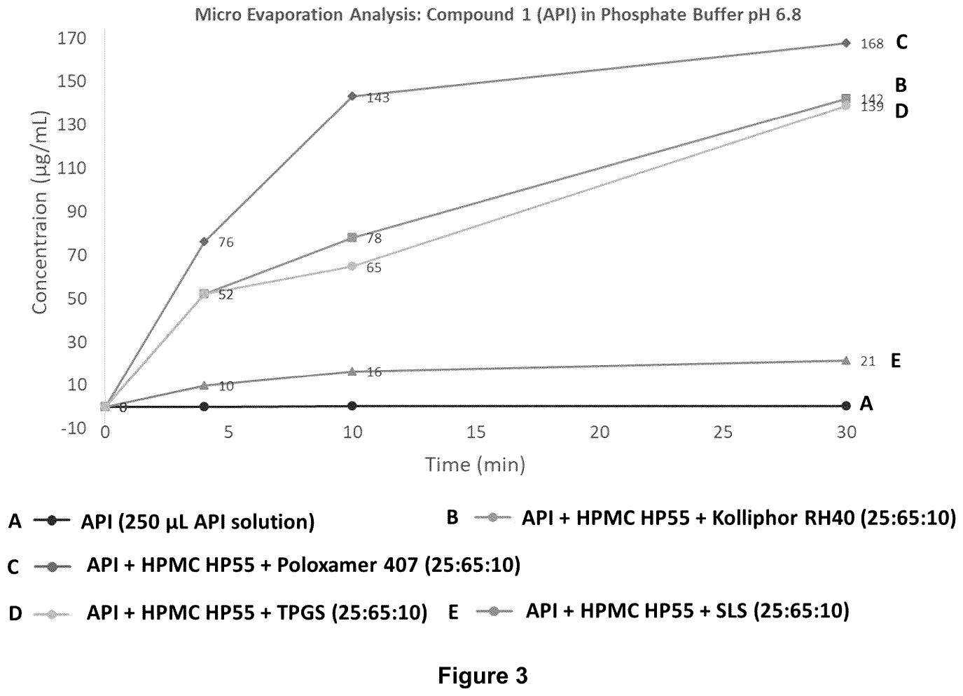

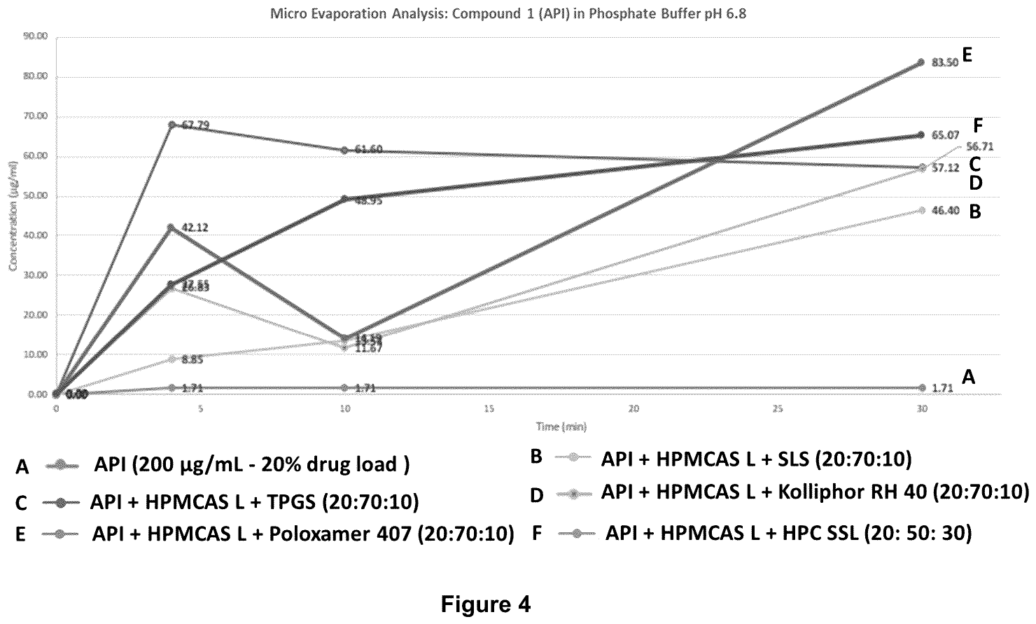

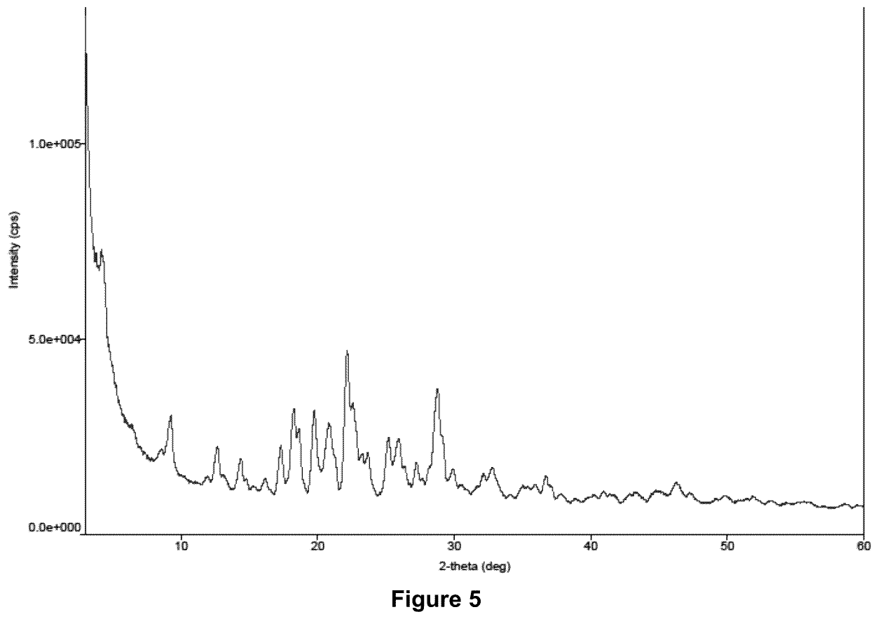

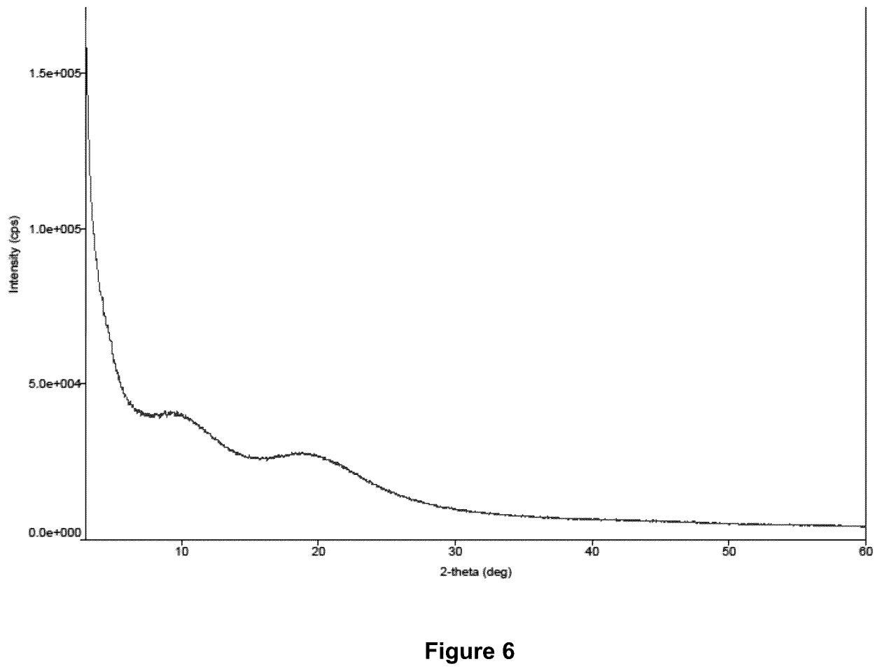

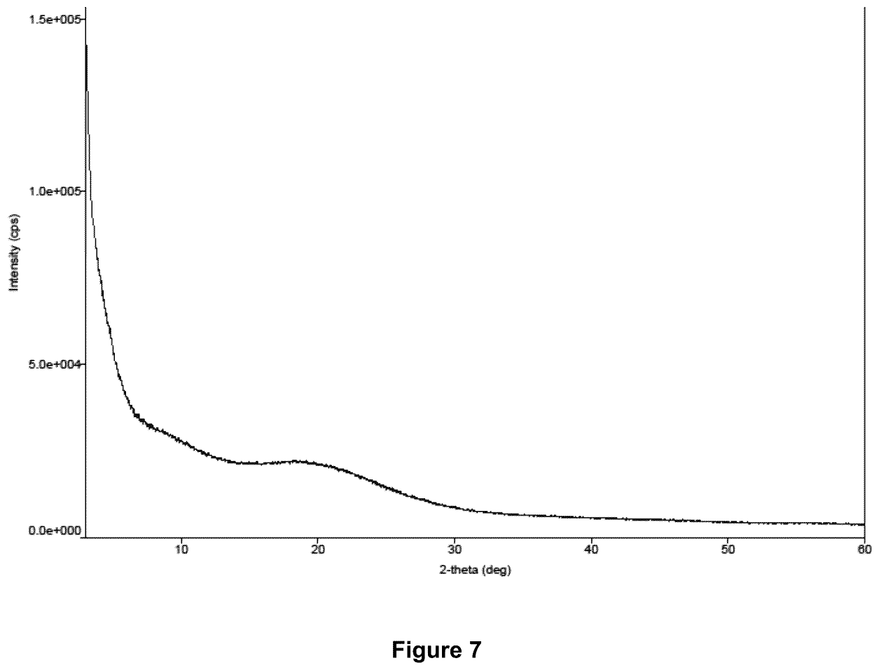

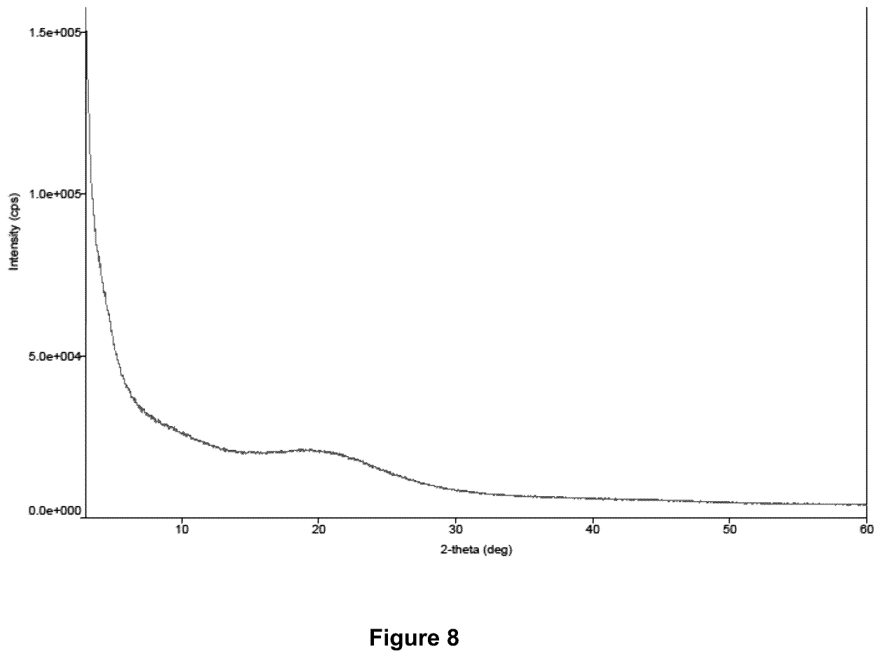

As shown in FIGS. 1-4, pharmaceutical compositions or formulations of Compound 1 (API) with either polymer or polymer/surfactant showed substantially improved re-dissolution behavior and a significant improvement in supersaturation concentration for the API. Compound 1 on its own showed high crystallinity in XRD analysis (FIG. 5) while various formulations of API with polymer or polymer/surfactant showed that the API is amorphous in pXRD analysis (FIGS. 6-8).

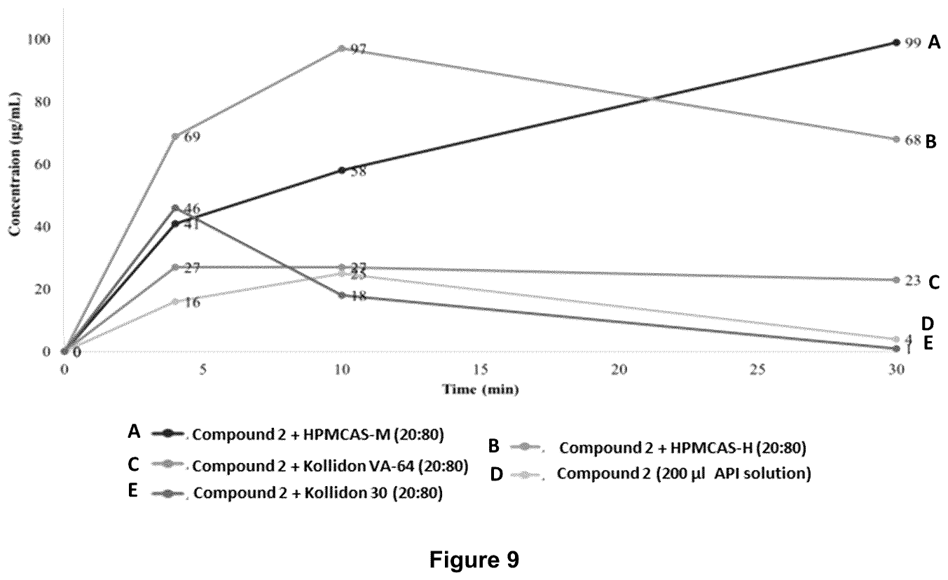

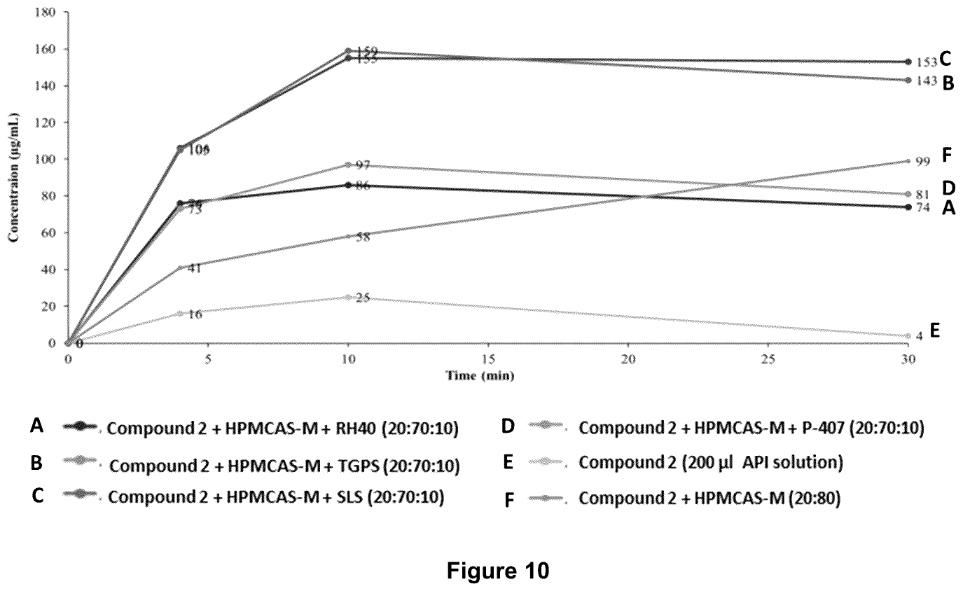

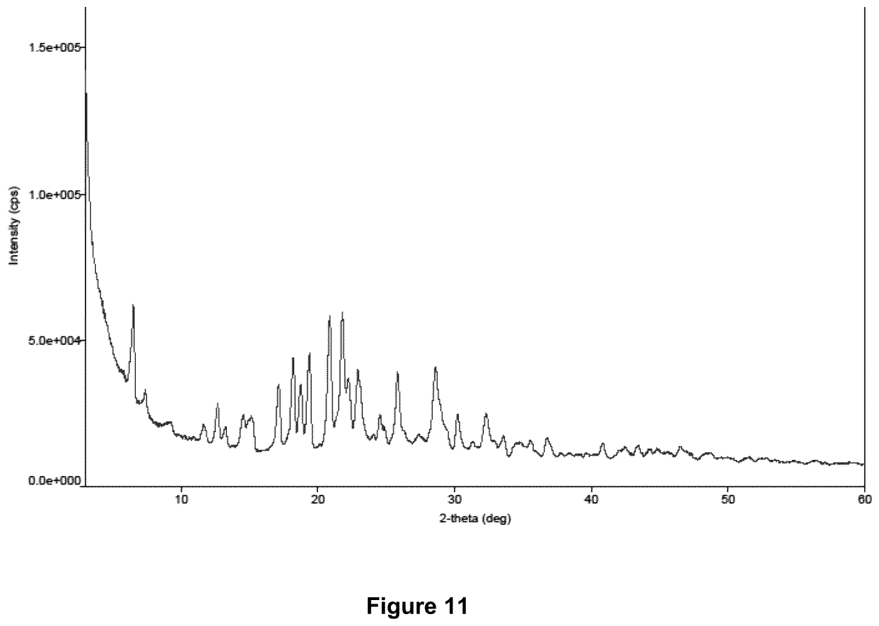

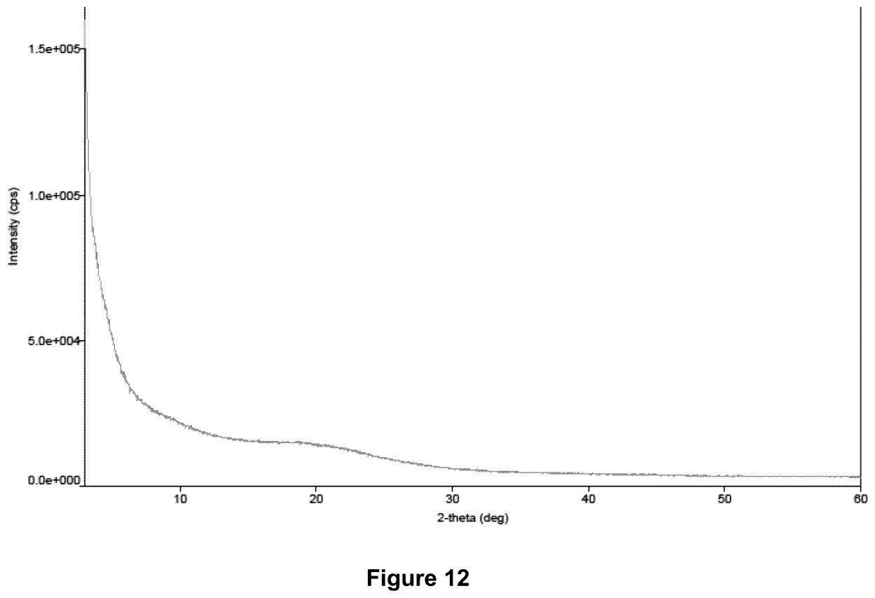

Pharmaceutical compositions or formulations of Compound 2 (API) with either polymer or polymer/surfactant also showed remarkable re-dissolution behavior and significant improvement in supersaturation concentration for the API (FIGS. 9-10). Compound 1 on its own showed high crystallinity in XRD analysis (FIG. 11) while the pharmaceutical compositions or formulations of the API showed that the API is amorphous in pXRD analysis (FIG. 12).

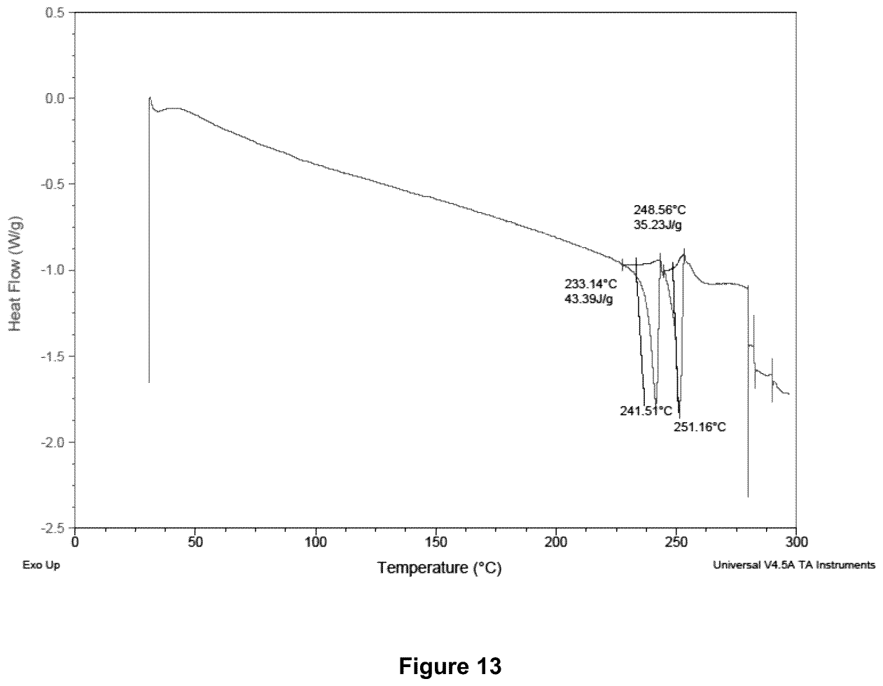

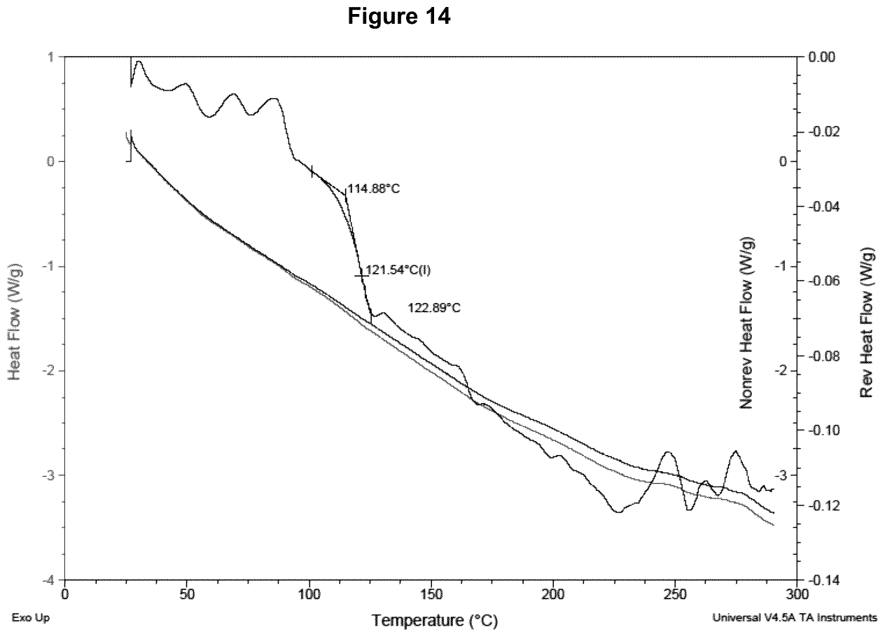

DSC thermogram for Compound 2 on its own shows two melting endotherms at 241° C. and 251° C. (FIG. 13). DSC thermogram for the pharmaceutical composition or formulations of Compound 2 in HPMC HP55 and Poloxamer 407 (25:65:10) thin film shows Tg at 121° C. confirming its feasibility towards HME (FIG. 14).

### Example 2

Mice (CD-1, weighing 25-30 g, n=4) are dosed using reconstitution vehicles intravenously (10% DMSO/70% PEG400/20% Water) or by oral gavage (1% Tween80 in 50 mM PBS (pH=6.8) alone or with lead formulations comprising of API+polymer+surfactant) with 30 mg/kg of the compound dissolved using the appropriate vehicle (concentration of 1 mg/mL with dosing volume of 10-30 ml/kg (PO) or 1-2 ml/kg (IV)). Drug load in the spray drying dispersion was maintained at ˜20% by weight for selected formulations. Time points include: 0.083 (IV only), 0.25, 0.5, 1, 2, 4, 8 and 24 hr. Plasma was extracted and analyzed using a Thermo-Finnigan Quantum Ultra triple quadrupole mass spectrometer with an Agilent 1100 HPLC front-end. Chromatographic separation is achieved using a Phenomenex Gemini column (2×50 mm, 5u). Hardware consists of a Finnigan TSQ Quantum Ultra triple quad mass spectrometer with both an APCI and ESI source and an Agilent 1100 front end. The mass spectrometer with mobile phase with 0.1% formic acid in water (A) and 0.1% formic acid in methanol (B).

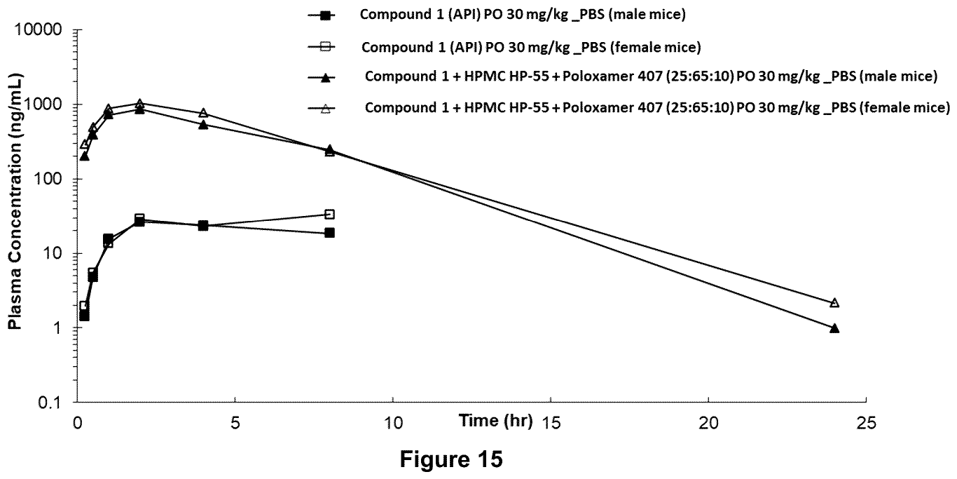

The pharmaceutical composition or formulation of Compound 1:HPMC HP-55:Poloxamer 407 (25:65:10) displayed an approximately 13-fold increase in AUCinf/D and an approximately 17-fold increase in oral bioavailability (F) compared to Compound 1 on its own (FIG. 15, Table 1).

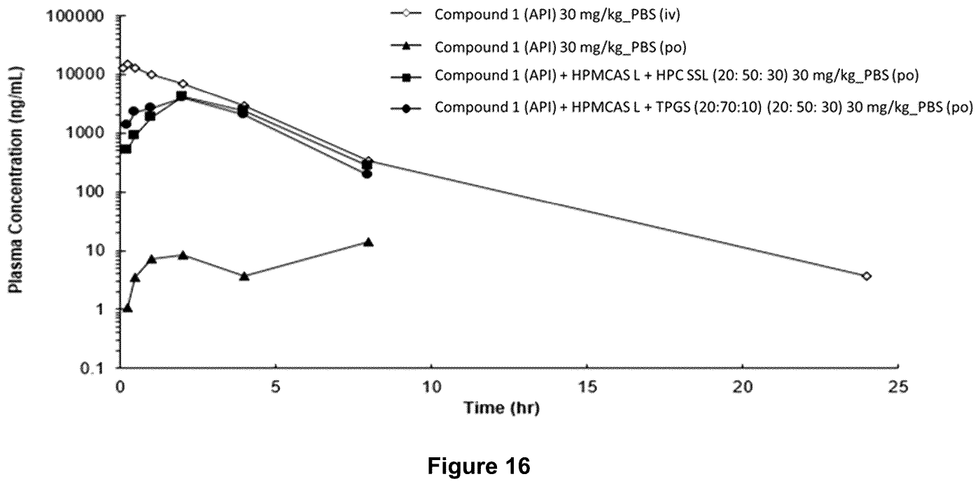

The pharmaceutical composition or formulation of Compound 1:HPMCAS L:HPC SSL (20:50:30) displayed more than a 300-fold increase in AUCinf/D and more than 280-fold increase in oral bioavailability (F) compared to Compound 2 on its own (FIG. 16, Table 2).

The pharmaceutical composition or formulation of Compound 1:HPMCAS L:TPGS (20:70:10) displayed more than a 300-fold increase in AUCinf/D and a more than a 280-fold increase in oral bioavailability (F) compared to Compound 2 on its own (FIG. 16, Table 2).

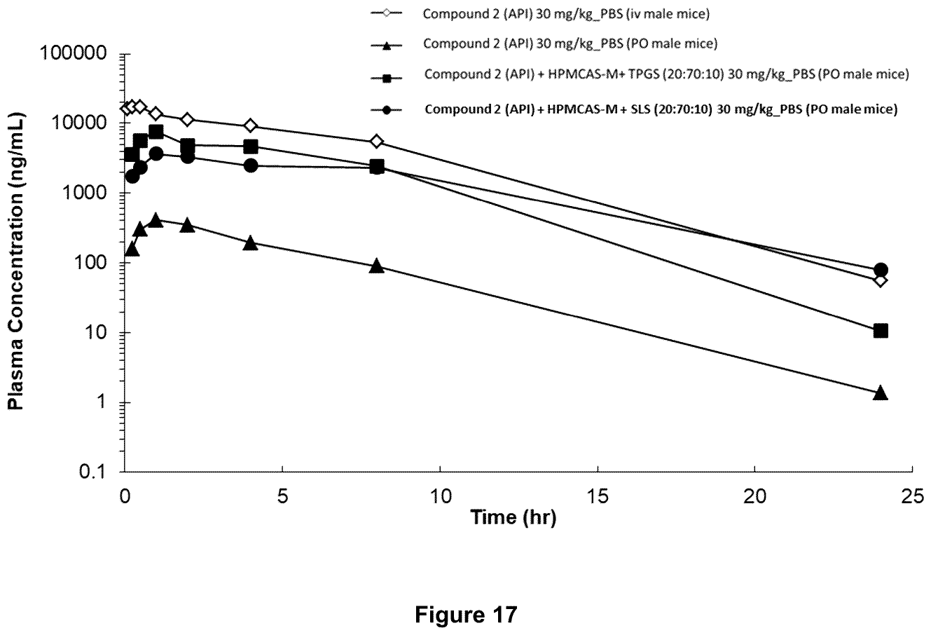

The pharmaceutical composition or formulation of Compound 2:HPMCAS-M:TPGS (20:70:10) displayed an approximately 22-fold increase in AUCinf/D and an approximately 22-fold increase in oral bioavailability (F) compared to Compound 2 on its own (FIG. 17, Table 3).

The formulation of Compound 2:HPMCAS-M:SLS (20:70:10) displayed an approximately 16.5-fold increase in AUCinf/D and an approximately 16.5-fold increase in oral bioavailability (F) compared to Compound 2 on its own (FIG. 17 and Table 3).

The examples and data included herein clearly establishes the efficacy of formulations including Compound 1 or Compound 2 in combination with a variety of polymers or a polymer/surfactant combinations in varying component concentrations for drug delivery. Those skilled in the art would readily understand that the inventive embodiments are not limited to the exact formulations disclosed herein. The specific formulations of the Experiments and accompanying data are merely exemplary working examples and themselves prophetic of the broader inventive scope of the formulations and methods described.

1. A pharmaceutical composition or formulation, comprising:
(i) a compound of Formula I or Formula II: 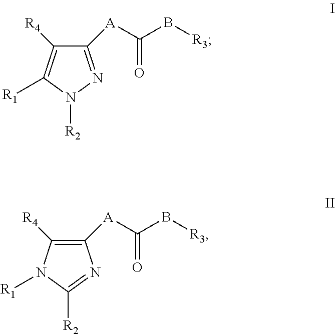
(ii) one or more pharmaceutically acceptable polymers;
(iii) optionally one or more pharmaceutically acceptable surfactants and/or one or more pharmaceutically acceptable surfactant-like materials;
(iv) optionally one or more pharmaceutically acceptable carriers and/or one or more pharmaceutically acceptable excipients; and
(v) optionally one or more solvents, in varying ratios, wherein
A is a direct bond,
B is N(H),
R1 is -T-(CH₂)ₘ-Q-(CH₂)ₙ—Z,
each of m and n is independently an integer from 0 to about 7;
T is selected from the group consisting of a carbocyclic ring having 3 to about 8 ring members, an unsaturated ring having 3 to about 8 carbon atoms as ring members, an aromatic ring having 5 to about 8 carbon atoms as ring members, a heterocyclic ring having 3 to about 8 ring members, a heteroaromatic ring having 5 to about 8 ring members, a bicyclic ring, a heterobicyclic ring, a tricyclic ring, a heterotricyclic ring, a polycyclic ring or a heteropolycyclic ring;
Q is CH═CH or C≡C;
Z is selected from the group consisting of H, halogen, CF₃, CF₂H, N₃, NCS, CN, NO₂, NX₁X₂, OX₃, SX₃, OAc, OSO₂X₃, O-acyl, S-acyl, SO₂-alkyl, SO-alkyl, SC(CH₃)₂COOX₈, OC(CH₃)₂COOX₈, C(CH₃)₂COOX₈, Si(alkyl)₃, alkyl-CN, O-aroyl, O(CH₂)ⱼOX₃, O(CH₂)ⱼNX₁X₂, NH-acyl, NH-aroyl, CHO, C(halogen)₃, COOX₃, SO₃H, SO₂NX₁X₂, CONX₁X₂, NHC(O)O-alkyl, NHSO2-alkyl, alkoxy, alkyl, alcohol, alkylmercapto, alkylamino, di-alkylamino, alkylsulfinyl, alkylsulfonyl, CX₄X₅X₆, —CH═CHX₈, and —C≡CX₈;
each of X₁ and X₂ is independently H or alkyl, or X₁ and X₂ together form part of a heterocyclic ring having about 4 to about 7 ring members and optionally one additional heteroatom selected from O, N and S, or
X₁ and X₂ together form part of an imide ring having about 5 to about 6 members,
X₃ is selected from the group consisting of H, alkyl, NO₂, (CH₂)ₘCN, hydroxyloweralkyl, and alkyl-NX₁X₂,
each of X₄, X₅, and X₆ is independently selected from the group consisting of H, alkyl, carbocyclic ring, hydroxyloweralkyl, alkyl-OH, halogen, CN, SNO, S(SO₂)alkyl, NX₁X₂, COOX₃, CONX₃, OX₇, and O-alkyl-X₇, wherein
X₇ is selected from the group consisting of H, alkyl, NO₂, NO, P(O)(OX₈)₂, PH(O)(OX₈), S(O)ₖN(alkyl)₂, S(O)ₖX₈, S(O)ₖOX₈, COOX₈, CONX₈, SO₃H and COX₈, wherein
X₈ is selected from the group consisting of H, alkyl, carbocyclic ring, heterocyclic ring, aromatic ring, heteroaromatic ring and —CX₉═CHX₁₀,
each of X₉ and X₁₀ is independently H or alkyl,
m is an integer from 0 to 7,
j is an integer from 0 to about 6, and
k is an integer from 0 to about 2;
R1 is -T-(CH₂)ₘ-Q-(CH₂)ₙ—Z;
each of m and n is independently an integer from 0 to about 7;
T is selected from the group consisting of a carbocyclic ring having 3 to about 8 ring members, an unsaturated ring having 3 to about 8 carbon atoms as ring members, an aromatic ring having 5 to about 8 carbon atoms as ring members, a heterocyclic ring having 3 to about 8 ring members, a heteroaromatic ring having 5 to about 8 ring members, a bicyclic ring, a heterobicyclic ring, a tricyclic ring, a heterotricyclic ring, a polycyclic ring and a heteropolycyclic ring;
Q is CH═CH or C≡C; and
Z is selected from the group consisting of a carbocyclic ring having about 4 to about 7 ring members, a heterocyclic ring having about 4 to about 7 ring members, an aromatic ring having about 5 to about 7 ring members, a heteroaromatic ring having about 5 to about 7 ring members, a bicyclic ring, a heterobicyclic ring, a tricyclic ring, a heterotricyclic ring, a polycyclic ring, a heteropolycyclic ring; or any above group substituted on at least one available ring atom by an alkyl group; or any above group substituted on at least one available ring nitrogen atom by a benzyl group, a substituted benzyl group, an alkoxybenzyl group, a substituted alkoxybenzyl group, a benzhydryl group or a substituted benzhydryl group; and wherein the connecting point between the —(CH₂)ₙ— group and the Z group can be any available ring carbon atom or any available ring nitrogen atom; or
R1 is -T-(CH₂)ₘ-Q-(CH₂)ₙ—Z;
each of m and n is independently an integer from 0 to about 7;
T is selected from the group consisting of a carbocyclic ring having 3 to about 8 ring members, an unsaturated ring having 3 to about 8 carbon atoms as ring members, an aromatic ring having 5 to about 8 carbon atoms as ring members, a heterocyclic ring having 3 to about 8 ring members, a heteroaromatic ring having 5 to about 8 ring members, a bicyclic ring, a heterobicyclic ring, a tricyclic ring, a heterotricyclic ring, a polycyclic ring and a heteropolycyclic ring;
Q is CH═CH or C≡C; and
Z is selected from the group consisting of 1-, 2- or 3-pyrrolidinyl, 1-, 2-, 3- or 4-piperidinyl, 2-, 3- or 4-morpholinyl, 2-, 3- or 4-thiomorpholinyl, 1-, 2- or 3-azetidinyl, 1- or 2-piperazinyl, 2- or 3-tetrahydrofuranyl; or any above group substituted on at least one available ring atom by an alkyl group; or any above group substituted on at least one available ring nitrogen atom by a benzyl group, a substituted benzyl group, an alkoxybenzyl group, a substituted alkoxybenzyl group, a benzhydryl group or a substituted benzhydryl group; and wherein the connecting point between the —(CH₂)ₙ— group and the Z group can be any available ring carbon atom or any available ring nitrogen atom; or
R1 is -T-(CH₂)ₘ-Q-(CH₂)ₙ—Z;
each of m and n is independently an integer from 0 to about 7;
T is selected from the group consisting of a carbocyclic ring having 3 to about 8 ring members, an unsaturated ring having 3 to about 8 carbon atoms as ring members, an aromatic ring having 5 to about 8 carbon atoms as ring members, a heterocyclic ring having 3 to about 8 ring members, a heteroaromatic ring having 5 to about 8 ring members, a bicyclic ring, a heterobicyclic ring, a tricyclic ring, a heterotricyclic ring, a polycyclic ring and a heteropolycyclic ring;
Q is CH═CH or C≡C; and
Z is selected from the group consisting of 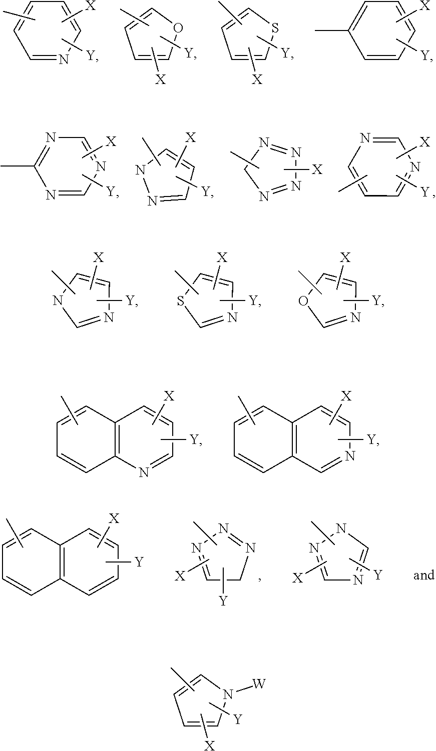
wherein each of X and Y is independently selected from the group consisting of H, halogen, CF₃, CF₂H, N₃, NCS, CN, NO₂, NX₁X₂, OX₃, SX₃, OAc, OSO₂X₃, O-acyl, S-acyl, SO₂-alkyl, SO-alkyl, SC(CH₃)₂COOX₈, OC(CH₃)₂COOX₈, C(CH₃)₂COOX₈, Si(alkyl)₃, alkyl-CN, O-aroyl, O(CH₂)ⱼOX₃, O(CH₂)ⱼNX₁X₂, NH-acyl, NH-aroyl, CHO, C(halogen)₃, COOX₃, SO₃H, SO₂NX₁X₂, CONX₁X₂, NHC(O)O-alkyl, NHSO2-alkyl, alkoxy, alkyl, alcohol, alkylmercapto, alkylamino, di-alkylamino, alkylsulfinyl or alkylsulfonyl, CX₄X₅X₆, —CH═CHX₈, and —C≡CX₈;
each of X₁ and X₂ is independently H or alkyl, or
X₁ and X₂ together form part of a heterocyclic ring having about 4 to about 7 ring members and optionally one additional heteroatom selected from O, N and S, or
X₁ and X₂ together form part of an imide ring having about 5 to about 6 members,
X₃ is selected from the group consisting of H, alkyl, NO₂, (CH₂)ₘCN, hydroxyloweralkyl, and alkyl-NX₁X₂,
each of X₄, X₅, and X₆ is independently selected from H, alkyl, carbocyclic ring, hydroxyloweralkyl, alkyl-OH, halogen, CN, SNO, S(SO₂)alkyl, NX₁X₂, COOX₃, CONX₃, OX₇, and O-alkyl-X₇, wherein
X₇ is selected from the group consisting of H, alkyl, NO₂, NO, P(O)(OX₈)₂, PH(O)(OX₈), S(O)ₖN(alkyl)₂, S(O)ₖX₈, S(O)ₖOX₈, COOX₈, CONX₈, SO₃H, and COX₈, wherein
X₈ is selected from the group consisting of H, alkyl, carbocyclic ring, heterocyclic ring, aromatic ring, heteroaromatic ring, and —CX₉═CHX₁₀, wherein
each of X₉ and X₁₀ is independently H or alkyl, wherein m is an integer from 0 to 7;
j is an integer from 0 to about 6,
k is an integer from 0 to about 2,
W is H or alkyl; or
R1 is -T-(CH₂)ₘ-Q-(CH₂)ₙ—Z;
each of m and n is independently an integer from 0 to about 7;
T is selected from the group consisting of a carbocyclic ring having 3 to about 8 ring members, an unsaturated ring having 3 to about 8 carbon atoms as ring members, an aromatic ring having 5 to about 8 carbon atoms as ring members, a heterocyclic ring having 3 to about 8 ring members, a heteroaromatic ring having 5 to about 8 ring members, a bicyclic ring, a heterobicyclic ring, a tricyclic ring, a heterotricyclic ring, a polycyclic ring and a heteropolycyclic ring;
Q is CH═CH or C≡C; and
Z is selected from the group consisting of an unsaturated ring having 5 ring atoms and 0 to 2 independently selected heteroatoms as ring members fused to an unsaturated ring having 5 ring atoms and 0 to 4 independently selected heteroatoms as ring members, an unsaturated ring having 5 ring atoms and 0 to 2 independently selected heteroatoms as ring members fused to an unsaturated ring having 6 or 7 ring atoms and 0 to 4 independently selected heteroatoms as ring members or an unsaturated ring having 6 ring atoms and 0 to 3 independently selected heteroatoms as ring members fused to an unsaturated ring having 6 or 7 ring atoms and 0 to 4 independently selected heteroatoms as ring members; or
R1 is -T-(CH₂)ₘ-Q-(CH₂)ₙ—Z;
T is selected from the group consisting of a carbocyclic ring having 3 to about 8 ring members, an unsaturated ring having 3 to about 8 carbon atoms as ring members, an aromatic ring having 5 to about 8 carbon atoms as ring members, a heterocyclic ring having 3 to about 8 ring members, a heteroaromatic ring having 5 to about 8 ring members, a bicyclic ring, a heterobicyclic ring, a tricyclic ring, a heterotricyclic ring, a polycyclic ring and a heteropolycyclic ring;
each of m and n is independently an integer from 0 to about 7;
Q is CH═CH or C≡C;
Z is 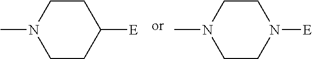
E is selected from the group consisting of a C1 to about C4, linear or branched alkyl group, a phenyl group, a substituted phenyl group, a benzyl group and a substituted benzyl group; or
R1 is -T-(CH₂)ₘ-Q-(CH₂)ₙ—Z;
T is selected from the group consisting of a carbocyclic ring having 3 to about 8 ring members, an unsaturated ring having 3 to about 8 carbon atoms as ring members, an aromatic ring having 5 to about 8 carbon atoms as ring members, a heterocyclic ring having 3 to about 8 ring members, a heteroaromatic ring having 5 to about 8 ring members, a bicyclic ring, a heterobicyclic ring, a tricyclic ring, a heterotricyclic ring, a polycyclic ring and a heteropolycyclic ring;
each of m and n is independently an integer from 0 to about 7;
Q is CH═CH or C≡C; and
Z is selected from the group consisting of 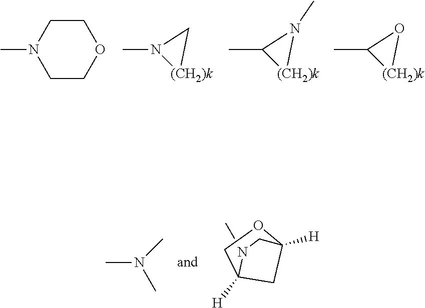
k is an integer from 1 to about 5, and
each of A₁ and A₂ is independently selected from the group consisting of a C1 to about C4 alkyl group, a phenyl group or a substituted phenyl group; or
R1 is —(CH₂)ₙ—Z;
n is an integer from 0 to about 7; wherein
Z is selected from the group consisting of H, halogen, CF₃, CF₂H, N₃, NCS, CN, NO₂, NX₁X₂, OX₃, SX₃, OAc, OSO₂X₃, O-acyl, S-acyl, SO₂-alkyl, SO-alkyl, SC(CH₃)₂COOX₈, OC(CH₃)₂COOX₈, C(CH₃)₂COOX₈, Si(alkyl)₃, alkyl-CN, O-aroyl, O(CH₂)ⱼOX₃, O(CH₂)ⱼNX₁X₂, NH-acyl, NH-aroyl, CHO, C(halogen)₃, COOX₃, SO₃H, SO₂NX₁X₂, CONX₁X₂, NHC(O)O-alkyl, NHSO2-alkyl, alkoxy, alkyl, alcohol, alkylmercapto, alkylamino, di-alkylamino, alkylsulfinyl or alkylsulfonyl, CX₄X₅X₆, —CH═CHX₈, and —C≡CX₈;
each of X₁ and X₂ is independently H or alkyl, or
X₁ and X₂ together form part of a heterocyclic ring having about 4 to about 7 ring members and optionally one additional heteroatom selected from O, N and S, or
X₁ and X₂ together form part of an imide ring having about 5 to about 6 members,
X₃ is selected from the group consisting of H, alkyl, aryl, NO₂, (CH₂)ₘCN, hydroxyloweralkyl, and alkyl-NX₁X₂,
each of X₄, X₅, and X₆ is independently selected from the group consisting of H, alkyl, carbocyclic ring, hydroxyloweralkyl, alkyl-OH, halogen, CN, SNO, S(SO₂)alkyl, NX₁X₂, COOX₃, CONX₃, OX₇, and O-alkyl-X₇, wherein
X₇ is selected from the group consisting of H, alkyl, NO₂, NO, P(O)(OX₈)₂, PH(O)(OX₈), S(O)ₖN(alkyl)₂, S(O)ₖX₈, S(O)ₖOX₈, COOX₈, CONX₈, SO₃H, and COX₈, wherein
X₈ is selected from the group consisting of H, alkyl, carbocyclic ring, heterocyclic ring, aromatic ring, heteroaromatic ring, and —CX₉═CHX₁₀, wherein
each of X₉ and X₁₀ is independently H or alkyl,
wherein m is an integer from 0 to 7,
j is an integer from 0 to about 6, or
k is an integer from 0 to about 2; or
R1 is —(CH₂)ₙ—Z;
n is an integer from 0 to about 7; and
Z is selected from the group consisting of a carbocyclic ring having about 4 to about 7 ring members, a heterocyclic ring having about 4 to about 7 ring members, an aromatic ring having about 5 to about 7 ring members, a heteroaromatic ring having about 5 to about 7 ring members, a bicyclic ring, a heterobicyclic ring, a tricyclic ring, a heterotricyclic ring, a polycyclic ring, a heteropolycyclic ring; or any above group substituted on at least one available ring atom by an alkyl group; or any above group substituted on at least one available ring nitrogen atom by a benzyl group, a substituted benzyl group, an alkoxybenzyl group, a substituted alkoxybenzyl group, a benzhydryl group or a substituted benzhydryl group; and wherein the connecting point between the —(CH₂)ₙ— group and the Z group can be any available ring carbon atom or any available ring nitrogen atom; or
R1 is —(CH₂)ₙ—Z;
n is an integer from 0 to about 7; and
Z is selected from the group consisting of a 5 member unsaturated ring having 0 to 4 independently selected heteroatoms as ring members, a substituted 5 member unsaturated ring having 0 to 4 independently selected heteroatoms as ring members, a 6 member aromatic ring having 0 to 5 independently selected heteroatoms as ring members or a substituted 6 member aromatic ring having 0 to 5 independently selected heteroatoms; and wherein the connecting point between the —(CH₂)ₙ— group and the Z group can be any available ring carbon atom or any available ring nitrogen atom; or
R1 is —(CH₂)ₙ—Z;
n is an integer from 0 to about 7; and
Z is selected from the group consisting of 1-, 2- or 3-pyrrolidinyl, 1-, 2-, 3- or 4-piperidinyl, 2-, 3- or 4-morpholinyl, 2-, 3- or 4-thiomorpholinyl, 1-, 2- or 3-azetidinyl, 1- or 2-piperazinyl, 2- or 3-tetrahydrofuranyl; or any above group substituted on at least one available ring atom by an alkyl group; or any above group substituted on at least one available ring nitrogen atom by a benzyl group, a substituted benzyl group, an alkoxybenzyl group, a substituted alkoxybenzyl group, a benzhydryl group or a substituted benzhydryl group; and wherein the connecting point between the —(CH₂)ₙ— group and the Z group can be any available ring carbon atom or any available ring nitrogen atom; or
R1 is —(CH₂)ₙ—Z;
n is an integer from 0 to about 7; and
Z is selected from the group consisting of 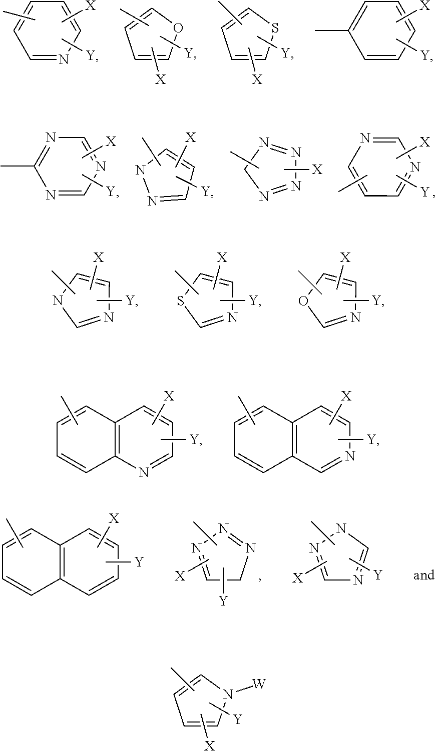
wherein each of X and Y is independently selected from the group consisting of, H, halogen, CF₃, CF₂H, N₃, NCS, CN, NO₂, NX₁X₂, OX₃, SX₃, OAc, OSO₂X₃, O-acyl, S-acyl, SO₂-alkyl, SO-alkyl, SC(CH₃)₂COOX₈, OC(CH₃)₂COOX₈, C(CH₃)₂COOX₈, Si(alkyl)₃, alkyl-CN, O-aroyl, O(CH₂)ⱼOX₃, O(CH₂)ⱼNX₁X₂, NH-acyl, NH-aroyl, CHO, C(halogen)₃, COOX₃, SO₃H, SO₂NX₁X₂, CONX₁X₂, NHC(O)O-alkyl, NHSO2-alkyl, alkoxy, alkyl, alcohol, alkylmercapto, alkylamino, di-alkylamino, alkylsulfinyl or alkylsulfonyl, CX₄X₅X₆, —CH═CHX₈, and —C≡CX₈;
each of X₁ and X₂ is independently H or alkyl, or
X₁ and X₂ together form part of a heterocyclic ring having about 4 to about 7 ring members and optionally one additional heteroatom selected from O, N and S, or
X₁ and X₂ together form part of an imide ring having about 5 to about 6 members,
X₃ is selected from the group consisting of H, alkyl, aryl, NO₂, (CH₂)ₘCN, hydroxyloweralkyl, and alkyl-NX₁X₂,
each of X₄, X₅, and X₆ is independently selected from H, alkyl, carbocyclic ring, hydroxyloweralkyl, alkyl-OH, halogen, CN, SNO, S(SO₂)alkyl, NX₁X₂, COOX₃, CONX₃, OX₇, and O-alkyl-X₇, wherein
X₇ is selected from the group consisting of H, alkyl, NO₂, NO, P(O)(OX₈)₂, PH(O)(OX₈), S(O)ₖN(alkyl)₂, S(O)ₖX₈, S(O)ₖOX₈, COOX₈, CONX₈, SO₃H, and COX₈, wherein
X₈ is selected from the group consisting of H, alkyl, carbocyclic ring, heterocyclic ring, aromatic ring, heteroaromatic ring, and —CX₉═CHX₁₀, wherein
each of X₉ and X₁₀ is independently H or alkyl,
wherein m is an integer from 0 to 7,
j is an integer from 0 to about 6, or
k is an integer from 0 to about 2
W is H or alkyl; or
R1 is —(CH₂)ₙ—Z;
n is an integer from 0 to about 7; and
Z is selected from the group consisting of a carbocyclic ring having 6 ring atoms fused to a heterocyclic ring having from 5 to 7 ring atoms, a carbocyclic ring having 6 ring atoms fused to a heteroaromatic ring having from 5 to 7 ring atoms, a heterocyclic ring having 6 ring atoms fused to a heterocyclic ring having from 5 to 7 ring atoms, an heterocyclic ring having 6 ring atoms fused to a heteroaromatic ring having from 5 to 7 ring atoms, an aromatic ring having 6 ring atoms fused to a heterocyclic ring having from 5 to 7 ring atoms, an aromatic ring having 6 ring atoms fused to a heteroaromatic ring having from 5 to 7 ring atoms, a heteroaromatic ring having 6 ring atoms fused to a heterocyclic ring having from 5 to 7 ring atoms or a heteroaromatic ring having 6 ring atoms fused to a heteroaromatic ring having from 5 to 7 ring atoms; or
R1 is —(CH₂)ₙ—Z;
n is an integer from 0 to about 7; and
Z is selected from the group consisting of an unsaturated ring having 5 ring atoms and 0 to 2 independently selected heteroatoms as ring members fused to an unsaturated ring having 5 ring atoms and 0 to 4 independently selected heteroatoms as ring members, an unsaturated ring having 5 ring atoms and 0 to 2 independently selected heteroatoms as ring members fused to an unsaturated ring having 6 or 7 ring atoms and 0 to 3 independently selected heteroatoms as ring members or an unsaturated ring having 6 ring atoms and 0 to 4 independently selected heteroatoms as ring members fused to an unsaturated ring having 6 or 7 ring atoms and 0 to 4 independently selected heteroatoms as ring members; or
R1 is —(CH₂)ₘ-Q₁-(CH₂)ₙ—Z;
Q₁ is selected from the group consisting of NH, O, S, —CH═CH—, —C≡C—, —CO, SO₂ and OSO₂;
m is an integer from 1 to about 7;
n is an integer from 0 to about 7; and
wherein Z is selected from the group consisting of H, halogen, CF₃, CF₂H, N₃, NCS, CN, NO₂, NX₁X₂, OX₃, SX₃, OAc, OSO₂X₃, O-acyl, S-acyl, SO₂-alkyl, SO-alkyl, SC(CH₃)₂COOX₈, OC(CH₃)₂COOX₈, C(CH₃)₂COOX₈, Si(alkyl)₃, alkyl-CN, O-aroyl, O(CH₂)ⱼOX₃, O(CH₂)ⱼNX₁X₂, NH-acyl, NH-aroyl, CHO, C(halogen)₃, COOX₃, SO₃H, SO₂NX₁X₂, CONX₁X₂, NHC(O)O-alkyl, NHSO2-alkyl, alkoxy, alkyl, alcohol, alkylmercapto, alkylamino, di-alkylamino, alkylsulfinyl or alkylsulfonyl, CX₄X₅X₆, —CH═CHX₈, and —C≡CX₈;
each of X₁ and X₂ is independently H or alkyl, or
X₁ and X₂ together form part of a heterocyclic ring having about 4 to about 7 ring members and optionally one additional heteroatom selected from O, N and S, or
X₁ and X₂ together form part of an imide ring having about 5 to about 6 members,
X₃ is selected from the group consisting of H, alkyl, aryl, NO₂, (CH₂)ₘCN, hydroxyloweralkyl, and alkyl-NX₁X₂,
X₄, X₅, and X₆ is independently selected from the group consisting of H, alkyl, carbocyclic ring, hydroxyloweralkyl, alkyl-OH, halogen, CN, SNO, S(SO₂)alkyl, NX₁X₂, COOX₃, CONX₃, OX₇, and O-alkyl-X₇ wherein
X₇ is selected from the group consisting of H, alkyl, NO₂, NO, P(O)(OX₈)₂, PH(O)(OX₈), S(O)ₖN(alkyl)₂, S(O)ₖX₈, S(O)ₖOX₈, COOX₈, CONX₈, SO₃H, and COX₈, wherein
X₈ is selected from the group consisting of H, alkyl, carbocyclic ring, heterocyclic ring, aromatic ring, heteroaromatic ring, and —CX₉═CHX₁₀, wherein
each of X₉ and X₁₀ is independently H or alkyl,
wherein m is an integer from 0 to 7,
j is an integer from 0 to about 6, or
k is an integer from 0 to about 2; or
R1 is -Q₂-(CH₂)ₙ—Z;
Q₂ is optionally present and if present is selected from the group consisting of —CH₂—NH, —CH₂—O, —CH₂—S, —CH₂—SO₂ and —CH₂—OSO₂;
n is an integer from 0 to about 7;
wherein Z is selected from the group consisting of H, halogen, CF₃, CF₂H, N₃, NCS, CN, NO₂, NX₁X₂, OX₃, SX₃, OAc, OSO₂X₃, O-acyl, S-acyl, SO₂-alkyl, SO-alkyl, SC(CH₃)₂COOX₈, OC(CH₃)₂COOX₈, C(CH₃)₂COOX₈, Si(alkyl)₃, alkyl-CN, O-aroyl, O(CH₂)ⱼOX₃, O(CH₂)ⱼNX₁X₂, NH-acyl, NH-aroyl, CHO, C(halogen)₃, COOX₃, SO₃H, SO₂NX₁X₂, CONX₁X₂, NHC(O)O-alkyl, NHSO2-alkyl, alkoxy, alkyl, alcohol, alkylmercapto, alkylamino, di-alkylamino, alkylsulfinyl or alkylsulfonyl, CX₄X₅X₆, —CH═CHX₈, and —C≡CX₈;
each of X₁ and X₂ is independently H or alkyl, or
X₁ and X₂ together form part of a heterocyclic ring having about 4 to about 7 ring members and optionally one additional heteroatom selected from O, N and S, or
X₁ and X₂ together form part of an imide ring having about 5 to about 6 members,
X₃ is selected from the group consisting of H, alkyl, aryl, NO₂, (CH₂)ₘCN, hydroxyloweralkyl, and alkyl-NX₁X₂,
each of X₄, X₅, and X₆ is independently selected from the group consisting of H, alkyl, carbocyclic ring, hydroxyloweralkyl, alkyl-OH, halogen, CN, SNO, S(SO₂)alkyl, NX₁X₂, COOX₃, CONX₃, OX₇, and O-alkyl-X₇ wherein
X₇ is selected from the group consisting of H, alkyl, NO₂, NO, P(O)(OX₈)₂, PH(O)(OX₈), S(O)ₖN(alkyl)₂, S(O)ₖX₈, S(O)ₖOX₈, COOX₈, CONX₈, SO₃H, and COX₈, wherein
X₈ is selected from the group consisting of H, alkyl, carbocyclic ring, heterocyclic ring, aromatic ring, heteroaromatic ring, and —CX₉═CHX₁₀, wherein
X₉ and X₁₀ is independently H or alkyl,
wherein m is an integer from 0 to 7,
j is an integer from 0 to about 6,
k is an integer from 0 to about 2; or
R1 is —(CH₂)ₘ-Q₁-(CH₂)ₙ—Z;
Q₁ is selected from the group consisting of NH, O, S, CH═CH, C≡C, CO, SO₂ and OSO₂;
m is an integer from 1 to about 7;
n is an integer from 0 to about 7;
Z is selected from the group consisting of a bicyclic ring, a heterobicyclic ring, a tricyclic ring, a heterotricyclic ring, a polycyclic ring and a heteropolycyclic ring; or
R1 is —(CH₂)ₘ-Q₁-(CH₂)ₙ—Z;
Q₁ is selected from the group consisting of NH, O, S, CH═CH, C≡C, CO, SO₂ and OSO₂;
m is an integer from 1 to about 7;
n is an integer from 0 to about 7;
Z is selected from the group consisting of a carbocyclic ring having about 4 to about 7 ring members, a heterocyclic ring having about 4 to about 7 ring members, an aromatic ring having about 5 to about 7 ring members, a heteroaromatic ring having about 5 to about 7 ring members; a bicyclic ring, a heterobicyclic ring, a tricyclic ring, a heterotricyclic ring, a polycyclic ring, a heteropolycyclic ring or any above group substituted on at least one available ring atom by an alkyl group or any above group substituted on at least one available ring nitrogen atom by a benzyl group, a substituted benzyl group, an alkoxybenzyl group, a substituted alkoxybenzyl group, a benzhydryl group or a substituted benzhydryl group; and wherein the connecting point between the —(CH₂)ₙ— group and the Z group can be any available ring carbon atom or any available ring nitrogen atom; or
R1 is —(CH₂)ₘ-Q₁-(CH₂)ₙ—Z;
Q₁ is selected from the group consisting of NH, O, S, CH═CH, C≡C, CO, SO₂ and OSO₂;
m is an integer from 1 to about 7;
n is an integer from 0 to about 7; and
Z is selected from the group consisting of a 5 member unsaturated ring having 0 to 4 independently selected heteroatoms as ring members, a substituted 5 member unsaturated ring having 0 to 4 independently selected heteroatoms as ring members, a 6 member aromatic ring having 0 to 5 independently selected heteroatoms as ring members or a substituted 6 member aromatic ring having 0 to 5 independently selected heteroatoms; and wherein the connecting point between the —(CH₂)ₙ— group and the Z group can be any available ring carbon atom or any available ring nitrogen atom; or
R1 is —(CH₂)ₘ-Q₁-(CH₂)ₙ—Z;
Q₁ is selected from the group consisting of NH, O, S, CH═CH, C≡C, CO, SO₂ and OSO₂;
m is an integer from 1 to about 7;
n is an integer from 0 to about 7;
Z is selected from the group consisting of 1-, 2- or 3-pyrrolidinyl, 1-, 2-, 3- or 4-piperidinyl, 2-, 3- or 4-morpholinyl, 2-, 3- or 4-thiomorpholinyl, 1-, 2- or 3-azetidinyl, 1- or 2-piperazinyl, 2- or 3-tetrahydrofuranyl; or any above group substituted on at least one available ring atom by an alkyl group; or any above group substituted on at least one available ring nitrogen atom by a benzyl group, a substituted benzyl group, an alkoxybenzyl group, a substituted alkoxybenzyl group, a benzhydryl group or a substituted benzhydryl group; and wherein the connecting point between the —(CH₂)ₙ— group and the Z group can be any available ring carbon atom or any available ring nitrogen atom; or
R1 is —(CH₂)ₘ-Q₁-(CH₂)ₙ—Z;
Q₁ is selected from the group consisting of NH, O, S, CH═CH, C≡C, CO, SO₂ and OSO₂;
m is an integer from 1 to about 7;
n is an integer from 0 to about 7; and
Z is selected from the group consisting of 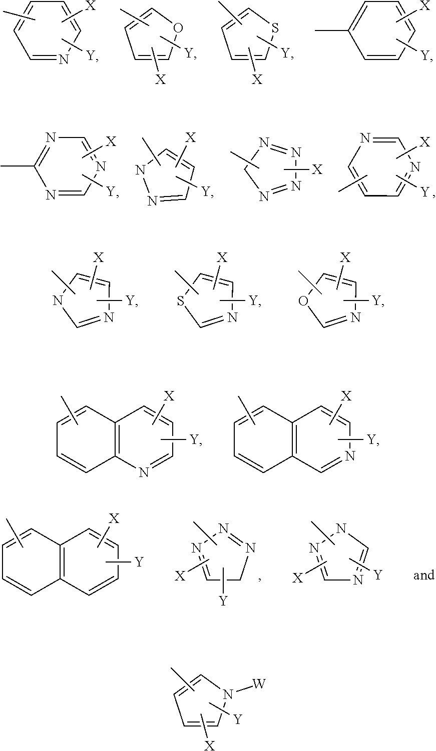
wherein each of X and Y is independently selected from the group consisting of H, halogen, CF₃, CF₂H, N₃, NCS, CN, NO₂, NX₁X₂, OX₃, SX₃, OAc, OSO₂X₃, O-acyl, S-acyl, SO₂-alkyl, SO-alkyl, SC(CH₃)₂COOX₈, OC(CH₃)₂COOX₈, C(CH₃)₂COOX₈, Si(alkyl)₃, alkyl-CN, O-aroyl, O(CH₂)ⱼOX₃, O(CH₂)ⱼNX₁X₂, NH-acyl, NH-aroyl, CHO, C(halogen)₃, COOX₃, SO₃H, SO₂NX₁X₂, CONX₁X₂, NHC(O)O-alkyl, NHSO2-alkyl, alkoxy, alkyl, alcohol, alkylmercapto, alkylamino, di-alkylamino, alkylsulfinyl or alkylsulfonyl, CX₄X₅X₆, —CH═CHX₈, and —C≡CX₈;
each of X₁ and X₂ is independently H or alkyl, or
X₁ and X₂ together form part of a heterocyclic ring having about 4 to about 7 ring members and optionally one additional heteroatom selected from O, N and S, or
X₁ and X₂ together form part of an imide ring having about 5 to about 6 members,
X₃ is selected from the group consisting of H, alkyl, aryl, NO₂, (CH₂)ₘCN, hydroxyloweralkyl, and alkyl-NX₁X₂,
X₄, X₅, and X₆ is independently selected from the group consisting of H, alkyl, carbocyclic ring, hydroxyloweralkyl, alkyl-OH, halogen, CN, SNO, S(SO₂)alkyl, NX₁X₂, COOX₃, CONX₃, OX₇, and O-alkyl-X₇, wherein
X₇ is selected from the group consisting of H, alkyl, NO₂, NO, P(O)(OX₈)₂, PH(O)(OX₈), S(O)ₖN(alkyl)₂, S(O)ₖX₈, S(O)ₖOX₈, COOX₈, CONX₈, SO₃H, and COX₈, wherein
X₈ is selected from the group consisting of H, alkyl, carbocyclic ring, heterocyclic ring, aromatic ring, heteroaromatic ring, and —CX₉═CHX₁₀, wherein
each of X₉ and X₁₀ is independently H or alkyl,
wherein m is an integer from 0 to 7,
j is an integer from 0 to about 6,
k is an integer from 0 to about 2,
W comprises H or alkyl; or
R1 is —(CH₂)ₘ-Q₁-(CH₂)ₙ—Z;
Q₁ is selected from the group consisting of NH, O, S, CH═CH, C≡C, CO, SO₂ and OSO₂;
m is an integer from 1 to about 7;
n is an integer from 0 to about 7; and
Z is selected from the group consisting of an unsaturated ring having 5 ring atoms and 0 to 2 independently selected heteroatoms as ring members fused to an unsaturated ring having 5 ring atoms and 0 to 4 independently selected heteroatoms as ring members, an unsaturated ring having 5 ring atoms and 0 to 2 independently selected heteroatoms as ring members fused to an unsaturated ring having 6 or 7 ring atoms and 0 to 4 independently selected heteroatoms as ring members or an unsaturated ring having 6 ring atoms and 0 to 4 independently selected heteroatoms as ring members fused to an unsaturated ring having 6 or 7 ring atoms and 0 to 4 independently selected heteroatoms as ring members; or
R1 is —(CH₂)ₘ-Q₁-(CH₂)ₙ—Z;
Q₁ is selected from the group consisting of NH, O, S, CH═CH, C≡C, CO, SO₂ and OSO₂;
m is an integer from 1 to about 7;
n is an integer from 0 to about 7; and
Z is selected from the group consisting of 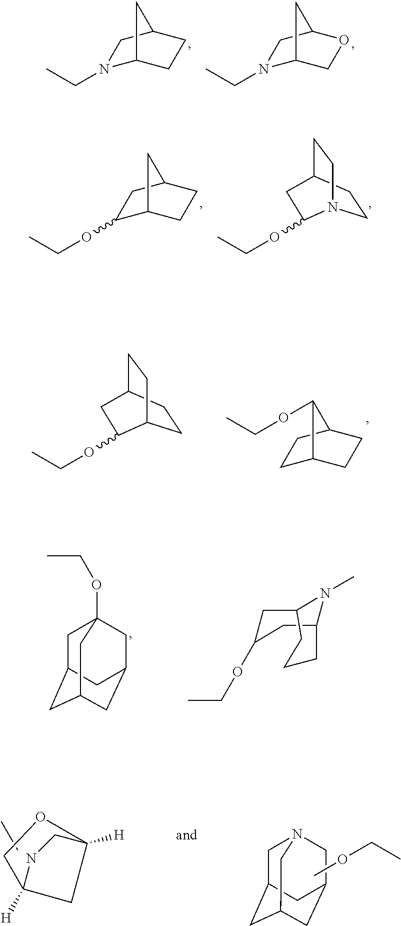
R1 is -T-(CH₂)ₙ—Z;
n is an integer from 0 to about 7;
T is selected from the group consisting of a carbocyclic ring having 3 to about 8 ring members, an unsaturated ring having 3 to about 8 carbon atoms as ring members, an aromatic ring having 5 to about 8 carbon atoms as ring members, a heterocyclic ring having 3 to about 8 ring members, a heteroaromatic ring having 5 to about 8 ring members, a bicyclic ring, a heterobicyclic ring, a tricyclic ring, a heterotricyclic ring, a polycyclic ring and a heteropolycyclic ring; and
Z is selected from the group consisting of, H, halogen, CF₃, CF₂H, N₃, NCS, CN, NO₂, NX₁X₂, OX₃, SX₃, OAc, OSO₂X₃, O-acyl, S-acyl, SO₂-alkyl, SO-alkyl, SC(CH₃)₂COOX₈, OC(CH₃)₂COOX₈, C(CH₃)₂COOX₈, Si(alkyl)₃, alkyl-CN, O-aroyl, O(CH₂)ⱼOX₃, O(CH₂)ⱼNX₁X₂, NH-acyl, NH-aroyl, CHO, C(halogen)₃, COOX₃, SO₃H, SO₂NX₁X₂, CONX₁X₂, NHC(O)O-alkyl, NHSO2-alkyl, alkoxy, alkyl, alcohol, alkylmercapto, alkylamino, di-alkylamino, alkylsulfinyl or alkylsulfonyl, CX₄X₅X₆, —CH═CHX₈, and —C≡CX₈;
each of X₁ and X₂ is independently H or alkyl, or
X₁ and X₂ together form part of a heterocyclic ring having about 4 to about 7 ring members and optionally one additional heteroatom selected from O, N and S, or
X₁ and X₂ together form part of an imide ring having about 5 to about 6 members,
X₃ is selected from the group consisting of H, alkyl, aryl, NO₂, (CH₂)ₘCN, hydroxyloweralkyl, and alkyl-NX₁X₂,
each of X₄, X₅, and X₆ is independently selected from the group consisting of H, alkyl, carbocyclic ring, hydroxyloweralkyl, alkyl-OH, halogen, CN, SNO, S(SO₂)alkyl, NX₁X₂, COOX₃, CONX₃, OX₇, and O-alkyl-X₇, wherein
X₇ is selected from the group consisting of H, alkyl, NO₂, NO, P(O)(OX₈)₂, PH(O)(OX₈), S(O)ₖN(alkyl)₂, S(O)ₖX₈, S(O)ₖOX₈, COOX₈, CONX₈, SO₃H, and COX₈, wherein
X₈ is selected from the group consisting of H, alkyl, carbocyclic ring, heterocyclic ring, aromatic ring, heteroaromatic ring, and —CX₉═CHX₁₀, wherein
each of X₉ and X₁₀ is independently H or alkyl,
wherein m is an integer from 0 to 7,
j is an integer from 0 to about 6,
k is an integer from 0 to about 2; or
R1 is -T-(CH₂)ₙ—Z;
n is an integer from 0 to about 7;
T is selected from the group consisting of a carbocyclic ring having 3 to about 8 ring members, an unsaturated ring having 3 to about 8 carbon atoms as ring members, an aromatic ring having 5 to about 8 carbon atoms as ring members, a heterocyclic ring having 3 to about 8 ring members, a heteroaromatic ring having 5 to about 8 ring members, a bicyclic ring, a heterobicyclic ring, a tricyclic ring, a heterotricyclic ring, a polycyclic ring and a heteropolycyclic ring; and
Z is selected from the group consisting of a carbocyclic ring having about 4 to about 7 ring members, a heterocyclic ring having about 4 to about 7 ring members, an aromatic ring having about 5 to about 7 ring members, a heteroaromatic ring having about 5 to about 7 ring members, a bicyclic ring, a heterobicyclic ring, a tricyclic ring, a heterotricyclic ring, a polycyclic ring, a heteropolycyclic ring; or any above group substituted on at least one available ring atom by an alkyl group; or any above group substituted on at least one available ring nitrogen atom by a benzyl group, a substituted benzyl group, an alkoxybenzyl group, a substituted alkoxybenzyl group, a benzhydryl group or a substituted benzhydryl group; and wherein the connecting point between the —(CH₂)ₙ— group and the Z group can be any available ring carbon atom or any available ring nitrogen atom; or
R1 is -T-(CH₂)ₙ—Z;
n is an integer from 0 to about 7;
T is selected from the group consisting of a carbocyclic ring having 3 to about 8 ring members, an unsaturated ring having 3 to about 8 carbon atoms as ring members, an aromatic ring having 5 to about 8 carbon atoms as ring members, a heterocyclic ring having 3 to about 8 ring members, a heteroaromatic ring having 5 to about 8 ring members, a bicyclic ring, a heterobicyclic ring, a tricyclic ring, a heterotricyclic ring, a polycyclic ring and a heteropolycyclic ring; and
Z is selected from the group consisting of 1-, 2- or 3-pyrrolidinyl, 1-, 2-, 3-azetidinyl, 1- or 2-piperazinyl, 2- or 3-tetrahydrofuranyl; or any above group substituted on at least one available ring atom by an alkyl group; or any above group substituted on at least one available ring nitrogen atom by a benzyl group, a substituted benzyl group, an alkoxybenzyl group, a substituted alkoxybenzyl group, a benzhydryl group or a substituted benzhydryl group; and wherein the connecting point between the —(CH₂)ₙ— group and the Z group can be any available ring carbon atom or any available ring nitrogen atom; or
R1 is -T-(CH₂)ₙ—Z;
n is an integer from 0 to about 7;
T is selected from the group consisting of a carbocyclic ring having 3 to about 8 ring members, an unsaturated ring having 3 to about 8 carbon atoms as ring members, an aromatic ring having 5 to about 8 carbon atoms as ring members, a heterocyclic ring having 3 to about 8 ring members, a heteroaromatic ring having 5 to about 8 ring members, a bicyclic ring, a heterobicyclic ring, a tricyclic ring, a heterotricyclic ring, a polycyclic ring and a heteropolycyclic ring; and
Z is selected from the group consisting of 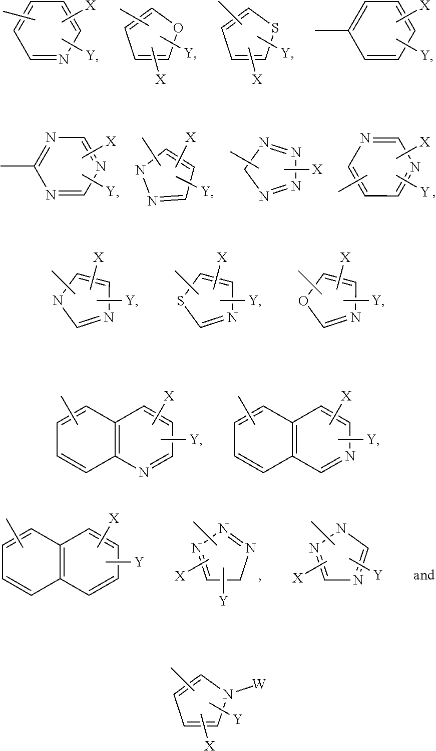
wherein X and Y are independently selected from the group consisting of H, halogen, CF₃, CF₂H, N₃, NCS, CN, NO₂, NX₁X₂, OX₃, SX₃, OAc, OSO₂X₃, O-acyl, S-acyl, SO₂-alkyl, SO-alkyl, SC(CH₃)₂COOX₈, OC(CH₃)₂COOX₈, C(CH₃)₂COOX₈, Si(alkyl)₃, alkyl-CN, O-aroyl, O(CH₂)ⱼOX₃, O(CH₂)ⱼNX₁X₂, NH-acyl, NH-aroyl, CHO, C(halogen)₃, COOX₃, SO₃H, SO₂NX₁X₂, CONX₁X₂, NHC(O)O-alkyl, NHSO2-alkyl, alkoxy, alkyl, alcohol, alkylmercapto, alkylamino, di-alkylamino, alkylsulfinyl or alkylsulfonyl, CX₄X₅X₆, —CH═CHX₈, and —C≡CX₈;
each of X₁ and X₂ is independently H or alkyl, or
X₁ and X₂ together form part of a heterocyclic ring having about 4 to about 7 ring members and optionally one additional heteroatom selected from O, N and S, or
X₁ and X₂ together form part of an imide ring having about 5 to about 6 members,
X₃ is selected from the group consisting of H, alkyl, aryl, NO₂, (CH₂)ₘCN, hydroxyloweralkyl, and alkyl-NX₁X₂,
each of X₄, X₅, and X₆ is independently selected from the group consisting of H, alkyl, carbocyclic ring, hydroxyloweralkyl, alkyl-OH, halogen, CN, SNO, S(SO₂)alkyl, NX₁X₂, COOX₃, CONX₃, OX₇, and O-alkyl-X₇, wherein
X₇ is selected from the group consisting of H, alkyl, NO₂, NO, P(O)(OX₈)₂, PH(O)(OX₈), S(O)ₖN(alkyl)₂, S(O)ₖX₈, S(O)ₖOX₈, COOX₈, CONX₈, SO₃H, and COX₈, wherein
X₈ is selected from the group consisting of H, alkyl, carbocyclic ring, heterocyclic ring, aromatic ring, heteroaromatic ring, and —CX₉═CHX₁₀, wherein
each of X₉ and X₁₀ is independently H or alkyl,
wherein m is an integer from 0 to 7,
j is an integer from 0 to about 6,
k is an integer from 0 to about 2
W is H or alkyl; or
R1 is -T-(CH₂)ₙ—Z;
n is an integer from 0 to about 7;
T is selected from the group consisting of a carbocyclic ring having 3 to about 8 ring members, an unsaturated ring having 3 to about 8 carbon atoms as ring members, an aromatic ring having 5 to about 8 carbon atoms as ring members, a heterocyclic ring having 3 to about 8 ring members, a heteroaromatic ring having 5 to about 8 ring members, a bicyclic ring, a heterobicyclic ring, a tricyclic ring, a heterotricyclic ring, a polycyclic ring and a heteropolycyclic ring; and
Z is selected from the group consisting of an unsaturated ring having 5 ring atoms and 0 to 2 independently selected heteroatoms as ring members fused to an unsaturated ring having 5 ring atoms and 0 to 4 independently selected heteroatoms as ring members, an unsaturated ring having 5 ring atoms and 0 to 2 independently selected heteroatoms as ring members fused to an unsaturated ring having 6 or 7 ring atoms and 0 to 4 independently selected heteroatoms as ring members or an unsaturated ring having 6 ring atoms and 0 to 3 independently selected heteroatoms as ring members fused to an unsaturated ring having 6 or 7 ring atoms and 0 to 4 independently selected heteroatoms as ring members; or
R1 is -T-Q-(CH₂)ₙ—Z,
each n is independently an integer from 0 to about 7;
T is selected from the group consisting of a carbocyclic ring having 3 to about 8 ring members, an unsaturated ring having 3 to about 8 carbon atoms as ring members, an aromatic ring having 5 to about 8 carbon atoms as ring members, a heterocyclic ring having 3 to about 8 ring members, a heteroaromatic ring having 5 to about 8 ring members, a bicyclic ring, a heterobicyclic ring, a tricyclic ring, a heterotricyclic ring, a polycyclic ring and a heteropolycyclic ring;
Q is CH═CH or C≡C;
Z is selected from the group consisting of H, halogen, CF₃, CF₂H, N₃, NCS, CN, NO₂, NX₁X₂, OX₃, SX₃, OAc, OSO₂X₃, O-acyl, S-acyl, SO₂-alkyl, SO-alkyl, SC(CH₃)₂COOX₈, OC(CH₃)₂COOX₈, C(CH₃)₂COOX₈, Si(alkyl)₃, alkyl-CN, O-aroyl, O(CH₂)ⱼOX₃, O(CH₂)ⱼNX₁X₂, NH-acyl, NH-aroyl, CHO, C(halogen)₃, COOX₃, SO₃H, SO₂NX₁X₂, CONX₁X₂, NHC(O)O-alkyl, NHSO2-alkyl, alkoxy, alkyl, alcohol, alkylmercapto, alkylamino, di-alkylamino, alkylsulfinyl or alkylsulfonyl, CX₄X₅X₆, —CH═CHX₈, and —C≡CX₈;
each of X₁ and X₂ is independently H or alkyl, or
X₁ and X₂ together form part of a heterocyclic ring having about 4 to about 7 ring members and optionally one additional heteroatom selected from O, N and S, or
X₁ and X₂ together form part of an imide ring having about 5 to about 6 members,
X₃ is selected from the group consisting of H, alkyl, NO₂, (CH₂)ₘCN, hydroxyloweralkyl, and alkyl-NX₁X₂,
each of X₄, X₅, and X₆ is independently selected from the group consisting of H, alkyl, carbocyclic ring, hydroxyloweralkyl, alkyl-OH, halogen, CN, SNO, S(SO₂)alkyl, NX₁X₂, COOX₃, CONX₃, OX₇, and O-alkyl-X₇, wherein
X₇ is selected from the group consisting of H, alkyl, NO₂, NO, P(O)(OX₈)₂, PH(O)(OX₈), S(O)ₖN(alkyl)₂, S(O)ₖX₈, S(O)ₖOX₈, COOX₈, CONX₈, SO₃H, and COX₈, wherein
X₈ is selected from the group consisting of H, alkyl, carbocyclic ring, heterocyclic ring, aromatic ring, heteroaromatic ring, and —CX₉═CHX₁₀, wherein
each of X₉ and X₁₀ is independently H or alkyl,
wherein m is an integer from 0 to 7,
j is an integer from 0 to about 6, or
k is an integer from 0 to about 2; or
R1 is -T-Q-(CH₂)ₙ—Z;
each n is independently an integer from 0 to about 7;
T is selected from the group consisting of a carbocyclic ring having 3 to about 8 ring members, an unsaturated ring having 3 to about 8 carbon atoms as ring members, an aromatic ring having 5 to about 8 carbon atoms as ring members, a heterocyclic ring having 3 to about 8 ring members, a heteroaromatic ring having 5 to about 8 ring members, a bicyclic ring, a heterobicyclic ring, a tricyclic ring, a heterotricyclic ring, a polycyclic ring and a heteropolycyclic ring;
Q is CH═CH or C≡C; and
Z is selected from the group consisting of a carbocyclic ring having about 4 to about 7 ring members, a heterocyclic ring having about 4 to about 7 ring members, an aromatic ring having about 5 to about 7 ring members, a heteroaromatic ring having about 5 to about 7 ring members, a bicyclic ring, a heterobicyclic ring, a tricyclic ring, a heterotricyclic ring, a polycyclic ring, a heteropolycyclic ring; or any above group substituted on at least one available ring atom by an alkyl group; or any above group substituted on at least one available ring nitrogen atom by a benzyl group, a substituted benzyl group, an alkoxybenzyl group, a substituted alkoxybenzyl group, a benzhydryl group or a substituted benzhydryl group; and wherein the connecting point between the —(CH₂)ₙ— group and the Z group can be any available ring carbon atom or any available ring nitrogen atom; or
R1 is -T-Q-(CH₂)ₙ—Z;
each n is independently an integer from 0 to about 7;
T is selected from the group consisting of a carbocyclic ring having 3 to about 8 ring members, an unsaturated ring having 3 to about 8 carbon atoms as ring members, an aromatic ring having 5 to about 8 carbon atoms as ring members, a heterocyclic ring having 3 to about 8 ring members, a heteroaromatic ring having 5 to about 8 ring members, a bicyclic ring, a heterobicyclic ring, a tricyclic ring, a heterotricyclic ring, a polycyclic ring and a heteropolycyclic ring;
Q is CH═CH or C≡C; and
Z is selected from the group consisting of 1-, 2- or 3-pyrrolidinyl, 1-, 2-, 3- or 4-piperidinyl, 2-, 3- or 4-morpholinyl, 2-, 3- or 4-thiomorpholinyl, 1-, 2- or 3-azetidinyl, 1- or 2-piperazinyl, 2- or 3-tetrahydrofuranyl; or any above group substituted on at least one available ring atom by an alkyl group; or any above group substituted on at least one available ring nitrogen atom by a benzyl group, a substituted benzyl group, an alkoxybenzyl group, a substituted alkoxybenzyl group, a benzhydryl group or a substituted benzhydryl group; and wherein the connecting point between the —(CH₂)ₙ— group and the Z group can be any available ring carbon atom or any available ring nitrogen atom; or
R1 is -T-Q-(CH₂)ₙ—Z;
each n is independently an integer from 0 to about 7;
T is selected from the group consisting of a carbocyclic ring having 3 to about 8 ring members, an unsaturated ring having 3 to about 8 carbon atoms as ring members, an aromatic ring having 5 to about 8 carbon atoms as ring members, a heterocyclic ring having 3 to about 8 ring members, a heteroaromatic ring having 5 to about 8 ring members, a bicyclic ring, a heterobicyclic ring, a tricyclic ring, a heterotricyclic ring, a polycyclic ring and a heteropolycyclic ring;
Q is CH═CH or C≡C; and
Z is selected from the group consisting of 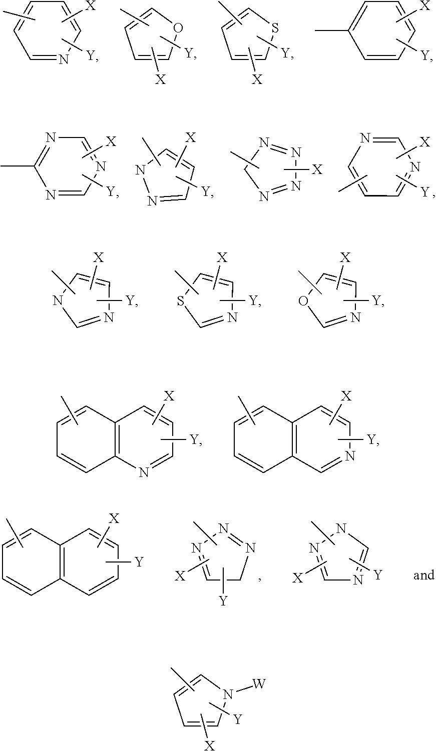
wherein X and Y are independently selected from the group consisting of H, halogen, CF₃, CF₂H, N₃, NCS, CN, NO₂, NX₁X₂, OX₃, SX₃, OAc, OSO₂X₃, O-acyl, S-acyl, SO₂-alkyl, SO-alkyl, SC(CH₃)₂COOX₈, OC(CH₃)₂COOX₈, C(CH₃)₂COOX₈, Si(alkyl)₃, alkyl-CN, O-aroyl, O(CH₂)ⱼOX₃, O(CH₂)ⱼNX₁X₂, NH-acyl, NH-aroyl, CHO, C(halogen)₃, COOX₃, SO₃H, SO₂NX₁X₂, CONX₁X₂, NHC(O)O-alkyl, NHSO2-alkyl, alkoxy, alkyl, alcohol, alkylmercapto, alkylamino, di-alkylamino, alkylsulfinyl or alkylsulfonyl, CX₄X₅X₆, —CH═CHX₈, and —C≡CX₈;
each of X₁ and X₂ is independently H or alkyl, or
X₁ and X₂ together form part of a heterocyclic ring having about 4 to about 7 ring members and optionally one additional heteroatom selected from O, N and S, or
X₁ and X₂ together form part of an imide ring having about 5 to about 6 members,
X₃ is selected from the group consisting of H, alkyl, NO₂, (CH₂)ₘCN, hydroxyloweralkyl, and alkyl-NX₁X₂,
each of X₄, X₅, and X₆ is independently selected from the group consisting of H, alkyl, carbocyclic ring, hydroxyloweralkyl, alkyl-OH, halogen, CN, SNO, S(SO₂)alkyl, NX₁X₂, COOX₃, CONX₃, OX₇, and O-alkyl-X₇, wherein
X₇ is selected from the group consisting of H, alkyl, NO₂, NO, P(O)(OX₈)₂, PH(O)(OX₈), S(O)ₖN(alkyl)₂, S(O)ₖX₈, S(O)ₖOX₈, COOX₈, CONX₈, SO₃H, and COX₈, wherein
X₈ is selected from the group consisting of H, alkyl, carbocyclic ring, heterocyclic ring, aromatic ring, heteroaromatic ring, and —CX₉═CHX₁₀, wherein
each of X₉ and X₁₀ is independently H or alkyl,
wherein m is an integer from 0 to 7,
j is an integer from 0 to about 6,
k is an integer from 0 to about 2,
W is H or alkyl; or
R1 is -T-Q-(CH₂)ₙ—Z;
each n is independently an integer from 0 to about 7;
T is selected from the group consisting of a carbocyclic ring having 3 to about 8 ring members, an unsaturated ring having 3 to about 8 carbon atoms as ring members, an aromatic ring having 5 to about 8 carbon atoms as ring members, a heterocyclic ring having 3 to about 8 ring members, a heteroaromatic ring having 5 to about 8 ring members, a bicyclic ring, a heterobicyclic ring, a tricyclic ring, a heterotricyclic ring, a polycyclic ring and a heteropolycyclic ring;
Q is CH═CH or C≡C; and
Z is selected from the group consisting of an unsaturated ring having 5 ring atoms and 0 to 2 independently selected heteroatoms as ring members fused to an unsaturated ring having 5 ring atoms and 0 to 4 independently selected heteroatoms as ring members, an unsaturated ring having 5 ring atoms and 0 to 2 independently selected heteroatoms as ring members fused to an unsaturated ring having 6 or 7 ring atoms and 0 to 4 independently selected heteroatoms as ring members or an unsaturated ring having 6 ring atoms and 0 to 3 independently selected heteroatoms as ring members fused to an unsaturated ring having 6 or 7 ring atoms and 0 to 4 independently selected heteroatoms as ring members; or
R1 is -T-Q-(CH₂)ₙ—Z;
T is selected from the group consisting of a carbocyclic ring having 3 to about 8 ring members, an unsaturated ring having 3 to about 8 carbon atoms as ring members, an aromatic ring having 5 to about 8 carbon atoms as ring members, a heterocyclic ring having 3 to about 8 ring members, a heteroaromatic ring having 5 to about 8 ring members, a bicyclic ring, a heterobicyclic ring, a tricyclic ring, a heterotricyclic ring, a polycyclic ring and a heteropolycyclic ring;
each n is independently an integer from 0 to about 7;
Q is CH═CH or C≡C;
Z is 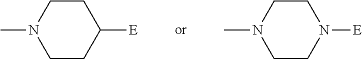
E is selected from the group consisting of a C1 to about C4, linear or branched alkyl group, a phenyl group, a substituted phenyl group, a benzyl group and a substituted benzyl group; or
R1 is -T-Q-(CH₂)ₙ—Z;
T is selected from the group consisting of a carbocyclic ring having 3 to about 8 ring members, an unsaturated ring having 3 to about 8 carbon atoms as ring members, an aromatic ring having 5 to about 8 carbon atoms as ring members, a heterocyclic ring having 3 to about 8 ring members, a heteroaromatic ring having 5 to about 8 ring members, a bicyclic ring, a heterobicyclic ring, a tricyclic ring, a heterotricyclic ring, a polycyclic ring and a heteropolycyclic ring;
each n is independently an integer from 0 to about 7;
Q is CH═CH or C≡C; and
Z is selected from the group consisting of 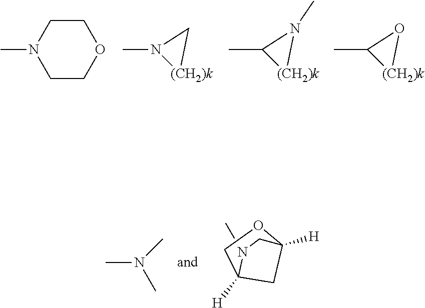
k is an integer from 1 to about 5, and
each of A₁ and A₂ is independently selected from the group consisting of a C1 to about C4 alkyl group, a phenyl group and a substituted phenyl group;
R2 is —(CH₂)ₙ—Z;
n is an integer from 0 to about 7; wherein
is selected from the group consisting of H, halogen, CF₃, CF₂H, N₃, NCS, CN, NO₂, NX₁X₂, OX₃, SX₃, OAc, OSO₂X₃, O-acyl, S-acyl, SO₂-alkyl, SO-alkyl, SC(CH₃)₂COOX₈, OC(CH₃)₂COOX₈, C(CH₃)₂COOX₈, Si(alkyl)₃, alkyl-CN, O-aroyl, O(CH₂)ⱼOX₃, O(CH₂)ⱼNX₁X₂, NH-acyl, NH-aroyl, CHO, C(halogen)₃, COOX₃, SO₃H, SO₂NX₁X₂, CONX₁X₂, NHC(O)O-alkyl, NHSO2-alkyl, alkoxy, alkyl, alcohol, alkylmercapto, alkylamino, di-alkylamino, alkylsulfinyl or alkylsulfonyl, CX₄X₅X₆, —CH═CHX₈, and —C≡CX₈;
each of X₁ and X₂ is independently H or alkyl, or
X₁ and X₂ together form part of a heterocyclic ring having about 4 to about 7 ring members and optionally one additional heteroatom selected from O, N and S, or
X₁ and X₂ together form part of an imide ring having about 5 to about 6 members,
X₃ is selected from the group consisting of H, alkyl, aryl, NO₂, (CH₂)ₘCN, hydroxyloweralkyl, and alkyl-NX₁X₂,
each of X₄, X₅, and X₆ is independently selected from the group consisting of H, alkyl, carbocyclic ring, hydroxyloweralkyl, alkyl-OH, halogen, CN, SNO, S(SO₂)alkyl, NX₁X₂, COOX₃, CONX₃, OX₇, and O-alkyl-X₇, wherein
X₇ is selected from the group consisting of H, alkyl, NO₂, NO, P(O)(OX₈)₂, PH(O)(OX₈), S(O)ₖN(alkyl)₂, S(O)ₖX₈, S(O)ₖOX₈, COOX₈, CONX₈, SO₃H and COX₈, wherein
X₈ is selected from the group consisting of H, alkyl, carbocyclic ring, heterocyclic ring, aromatic ring, heteroaromatic ring, and —CX₉═CHX₁₀, wherein
each of X₉ and X₁₀ is independently H or alkyl,
wherein m is an integer from 0 to 7,
j is an integer from 0 to about 6, or
k is an integer from 0 to about 2; or
R2 is —(CH₂)ₙ—Z;
n is an integer from 0 to about 7; and
Z is selected from the group consisting of a carbocyclic ring having about 4 to about 7 ring members, a heterocyclic ring having about 4 to about 7 ring members, an aromatic ring having about 5 to about 7 ring members, a heteroaromatic ring having about 5 to about 7 ring members, a bicyclic ring, a heterobicyclic ring, a tricyclic ring, a heterotricyclic ring, a polycyclic ring, a heteropolycyclic ring; or any above group substituted on at least one available ring atom by an alkyl group; or any above group substituted on at least one available ring nitrogen atom by a benzyl group, a substituted benzyl group, an alkoxybenzyl group, a substituted alkoxybenzyl group, a benzhydryl group or a substituted benzhydryl group; and wherein the connecting point between the —(CH₂)ₙ— group and the Z group can be any available ring carbon atom or any available ring nitrogen atom; or
R2 is —(CH₂)ₙ—Z;
n is an integer from 0 to about 7; and
Z is selected from the group consisting of a 5 member unsaturated ring having 0 to 4 independently selected heteroatoms as ring members, a substituted 5 member unsaturated ring having 0 to 4 independently selected heteroatoms as ring members, a 6 member aromatic ring having 0 to 5 independently selected heteroatoms as ring members or a substituted 6 member aromatic ring having 0 to 5 independently selected heteroatoms; and wherein the connecting point between the —(CH₂)ₙ— group and the Z group can be any available ring carbon atom or any available ring nitrogen atom; or
R2 is —(CH₂)ₙ—Z;
n is an integer from 0 to about 7; and
Z is selected from the group consisting of 1-, 2- or 3-pyrrolidinyl, 1-, 2-, 3- or 4-piperidinyl, 2-, 3- or 4-morpholinyl, 2-, 3- or 4-thiomorpholinyl, 1-, 2- or 3-azetidinyl, 1- or 2-piperazinyl, 2- or 3-tetrahydrofuranyl; or any above group substituted on at least one available ring atom by an alkyl group; or any above group substituted on at least one available ring nitrogen atom by a benzyl group, a substituted benzyl group, an alkoxybenzyl group, a substituted alkoxybenzyl group, a benzhydryl group or a substituted benzhydryl group; and wherein the connecting point between the —(CH₂)ₙ— group and the Z group can be any available ring carbon atom or any available ring nitrogen atom; or
R2 is —(CH₂)ₙ—Z;
n is an integer from 0 to about 7; and
Z is selected from the group consisting of 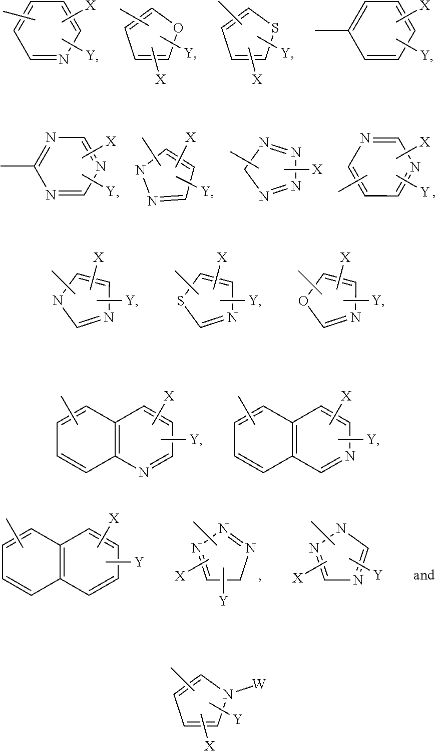
wherein each of X and Y is independently selected from the group consisting of H, halogen, CF₃, CF₂H, N₃, NCS, CN, NO₂, NX₁X₂, OX₃, SX₃, OAc, OSO₂X₃, O-acyl, S-acyl, SO₂-alkyl, SO-alkyl, SC(CH₃)₂COOX₈, OC(CH₃)₂COOX₈, C(CH₃)₂COOX₈, Si(alkyl)₃, alkyl-CN, O-aroyl, O(CH₂)ⱼOX₃, O(CH₂)ⱼNX₁X₂, NH-acyl, NH-aroyl, CHO, C(halogen)₃, COOX₃, SO₃H, SO₂NX₁X₂, CONX₁X₂, NHC(O)O-alkyl, NHSO2-alkyl, alkoxy, alkyl, alcohol, alkylmercapto, alkylamino, di-alkylamino, alkylsulfinyl or alkylsulfonyl, CX₄X₅X₆, —CH═CHX₈, and —C≡CX₈;
each of X₁ and X₂ is independently H or alkyl, or
X₁ and X₂ together form part of a heterocyclic ring having about 4 to about 7 ring members and optionally one additional heteroatom selected from O, N and S, or
X₁ and X₂ together form part of an imide ring having about 5 to about 6 members,
X₃ is selected from the group consisting of H, alkyl, aryl, NO₂, (CH₂)ₘCN, hydroxyloweralkyl, and alkyl-NX₁X₂,
each of X₄, X₅, and X₆ is independently selected from the group consisting of H, alkyl, carbocyclic ring, hydroxyloweralkyl, alkyl-OH, halogen, CN, SNO, S(SO₂)alkyl, NX₁X₂, COOX₃, CONX₃, OX₇, and O-alkyl-X₇, wherein
X₇ is selected from the group consisting of H, alkyl, NO₂, NO, P(O)(OX₈)₂, PH(O)(OX₈), S(O)ₖN(alkyl)₂, S(O)ₖX₈, S(O)ₖOX₈, COOX₈, CONX₈, SO₃H, and COX₈, wherein
X₈ is selected from the group consisting of H, alkyl, carbocyclic ring, heterocyclic ring, aromatic ring, heteroaromatic ring, and —CX₉═CHX₁₀, wherein
each of X₉ and X₁₀ is independently H or alkyl,
wherein m is an integer from 0 to 7,
j is an integer from 0 to about 6, or
k is an integer from 0 to about 2,
W comprises H or alkyl; or
R2 is —(CH₂)ₙ—Z;
n is an integer from 0 to about 7; and
Z is selected from the group consisting of a carbocyclic ring having 6 ring atoms fused to a heterocyclic ring having from 5 to 7 ring atoms, a carbocyclic ring having 6 ring atoms fused to a heteroaromatic ring having from 5 to 7 ring atoms, a heterocyclic ring having 6 ring atoms fused to a heterocyclic ring having from 5 to 7 ring atoms, an heterocyclic ring having 6 ring atoms fused to a heteroaromatic ring having from 5 to 7 ring atoms, an aromatic ring having 6 ring atoms fused to a heterocyclic ring having from 5 to 7 ring atoms, an aromatic ring having 6 ring atoms fused to a heteroaromatic ring having from 5 to 7 ring atoms, a heteroaromatic ring having 6 ring atoms fused to a heterocyclic ring having from 5 to 7 ring atoms or a heteroaromatic ring having 6 ring atoms fused to a heteroaromatic ring having from 5 to 7 ring atoms; or
R2 is —(CH₂)ₙ—Z;
n is an integer from 0 to about 7; and
Z is selected from the group consisting of an unsaturated ring having 5 ring atoms and 0 to 2 independently selected heteroatoms as ring members fused to an unsaturated ring having 5 ring atoms and 0 to 4 independently selected heteroatoms as ring members, an unsaturated ring having 5 ring atoms and 0 to 2 independently selected heteroatoms as ring members fused to an unsaturated ring having 6 or 7 ring atoms and 0 to 3 independently selected heteroatoms as ring members or an unsaturated ring having 6 ring atoms and 0 to 4 independently selected heteroatoms as ring members fused to an unsaturated ring having 6 or 7 ring atoms and 0 to 4 independently selected heteroatoms as ring members; or
R2 is —(CH₂)ₘ-Q₁-(CH₂)ₙ—Z;
Q₁ is selected from the group consisting of NH, O, S, —CH═CH—, —C≡C—, —CO, SO₂ and OSO₂;
m is an integer from 1 to about 7;
n is an integer from 0 to about 7; and
wherein Z is selected from the group consisting of H, halogen, CF₃, CF₂H, N₃, NCS, CN, NO₂, NX₁X₂, OX₃, SX₃, OAc, OSO₂X₃, O-acyl, S-acyl, SO₂-alkyl, SO-alkyl, SC(CH₃)₂COOX₈, OC(CH₃)₂COOX₈, C(CH₃)₂COOX₈, Si(alkyl)₃, alkyl-CN, O-aroyl, O(CH₂)ⱼOX₃, O(CH₂)ⱼNX₁X₂, NH-acyl, NH-aroyl, CHO, C(halogen)₃, COOX₃, SO₃H, SO₂NX₁X₂, CONX₁X₂, NHC(O)O-alkyl, NHSO2-alkyl, alkoxy, alkyl, alcohol, alkylmercapto, alkylamino, di-alkylamino, alkylsulfinyl or alkylsulfonyl, CX₄X₅X₆, —CH═CHX₈, and —C≡CX₈;
each of X₁ and X₂ is independently H or alkyl, or
X₁ and X₂ together form part of a heterocyclic ring having about 4 to about 7 ring members and optionally one additional heteroatom selected from O, N and S, or
X₁ and X₂ together form part of an imide ring having about 5 to about 6 members,
X₃ is selected from the group consisting of H, alkyl, aryl, NO₂, (CH₂)ₘCN, hydroxyloweralkyl, and alkyl-NX₁X₂,
each of X₄, X₅, and X₆ is independently selected from the group consisting of H, alkyl, carbocyclic ring, hydroxyloweralkyl, alkyl-OH, halogen, CN, SNO, S(SO₂)alkyl, NX₁X₂, COOX₃, CONX₃, OX₇, and O-alkyl-X₇ wherein
X₇ is selected from the group consisting of H, alkyl, NO₂, NO, P(O)(OX₈)₂, PH(O)(OX₈), S(O)ₖN(alkyl)₂, S(O)ₖX₈, S(O)ₖOX₈, COOX₈, CONX₈, SO₃H, and COX₈, wherein
X₈ is selected from the group consisting of H, alkyl, carbocyclic ring, heterocyclic ring, aromatic ring, heteroaromatic ring, and —CX₉═CHX₁₀, wherein
each of X₉ and X₁₀ is independently H or alkyl,
wherein m is an integer from 0 to 7,
j is an integer from 0 to about 6,
k is an integer from 0 to about 2; or
R2 is -Q₂-(CH₂)ₙ—Z;
Q₂ is optionally present and if present is selected from the group consisting of —CH₂—NH, —CH₂—O, —CH₂—S, —CH₂—SO₂ and —CH₂—OSO₂;
n is an integer from 0 to about 7;
wherein Z is selected from the group consisting of H, halogen, CF₃, CF₂H, N₃, NCS, CN, NO₂, NX₁X₂, OX₃, SX₃, OAc, OSO₂X₃, O-acyl, S-acyl, SO₂-alkyl, SO-alkyl, SC(CH₃)₂COOX₈, OC(CH₃)₂COOX₈, C(CH₃)₂COOX₈, Si(alkyl)₃, alkyl-CN, O-aroyl, O(CH₂)ⱼOX₃, O(CH₂)ⱼNX₁X₂, NH-acyl, NH-aroyl, CHO, C(halogen)₃, COOX₃, SO₃H, SO₂NX₁X₂, CONX₁X₂, NHC(O)O-alkyl, NHSO2-alkyl, alkoxy, alkyl, alcohol, alkylmercapto, alkylamino, di-alkylamino, alkylsulfinyl or alkylsulfonyl, CX₄X₅X₆, —CH═CHX₈, and —C≡CX₈;
each of X₁ and X₂ is independently H or alkyl, or
X₁ and X₂ together form part of a heterocyclic ring having about 4 to about 7 ring members and optionally one additional heteroatom selected from O, N and S, or
X₁ and X₂ together form part of an imide ring having about 5 to about 6 members,
X₃ is selected from the group consisting of H, alkyl, aryl, NO₂, (CH₂)ₘCN, hydroxyloweralkyl, and alkyl-NX₁X₂,
each of X₄, X₅, and X₆ is independently selected from the group consisting of H, alkyl, carbocyclic ring, hydroxyloweralkyl, alkyl-OH, halogen, CN, SNO, S(SO₂)alkyl, NX₁X₂, COOX₃, CONX₃, OX₇, and O-alkyl-X₇, wherein
X₇ is selected from the group consisting of H, alkyl, NO₂, NO, P(O)(OX₈)₂, PH(O)(OX₈), S(O)ₖN(alkyl)₂, S(O)ₖX₈, S(O)ₖOX₈, COOX₈, CONX₈, SO₃H, and COX₈, wherein
X₈ is selected from the group consisting of H, alkyl, carbocyclic ring, heterocyclic ring, aromatic ring, heteroaromatic ring, and —CX₉═CHX₁₀, wherein
each of X₉ and X₁₀ is independently H or alkyl,
wherein m is an integer from 0 to 7,
j is an integer from 0 to about 6,
k is an integer from 0 to about 2; or
R2 is —(CH₂)ₘ-Q₁-(CH₂)ₙ—Z;
Q₁ is selected from the group consisting of NH, O, S, CH═CH, C≡C, CO, SO₂ and OSO₂;
m is an integer from 1 to about 7;
n is an integer from 0 to about 7;
Z is selected from the group consisting of a bicyclic ring, a heterobicyclic ring, a tricyclic ring, a heterotricyclic ring, a polycyclic ring and a heteropolycyclic ring; or
R2 is —(CH₂)ₘ-Q₁-(CH₂)ₙ—Z;
Q₁ is selected from the group consisting of NH, O, S, CH═CH, C≡C, CO, SO₂ and OSO₂;
m is an integer from 1 to about 7;
n is an integer from 0 to about 7;
Z is selected from the group consisting of a carbocyclic ring having about 4 to about 7 ring members, a heterocyclic ring having about 4 to about 7 ring members, an aromatic ring having about 5 to about 7 ring members, a heteroaromatic ring having about 5 to about 7 ring members; a bicyclic ring, a heterobicyclic ring, a tricyclic ring, a heterotricyclic ring, a polycyclic ring, a heteropolycyclic ring or any above group substituted on at least one available ring atom by an alkyl group or any above group substituted on at least one available ring nitrogen atom by a benzyl group, a substituted benzyl group, an alkoxybenzyl group, a substituted alkoxybenzyl group, a benzhydryl group or a substituted benzhydryl group; and wherein the connecting point between the —(CH₂)ₙ— group and the Z group can be any available ring carbon atom or any available ring nitrogen atom; or
R2 is —(CH₂)ₘ-Q₁-(CH₂)ₙ—Z;
Q₁ is selected from the group consisting of NH, O, S, CH═CH, C≡C, CO, SO₂ and OSO₂;
m is an integer from 1 to about 7;
n is an integer from 0 to about 7; and
Z is selected from the group consisting of a 5 member unsaturated ring having 0 to 4 independently selected heteroatoms as ring members, a substituted 5 member unsaturated ring having 0 to 4 independently selected heteroatoms as ring members, a 6 member aromatic ring having 0 to 5 independently selected heteroatoms as ring members or a substituted 6 member aromatic ring having 0 to 5 independently selected heteroatoms; and wherein the connecting point between the —(CH₂)ₙ— group and the Z group can be any available ring carbon atom or any available ring nitrogen atom; or
R2 is —(CH₂)ₘ-Q₁-(CH₂)ₙ—Z;
Q₁ is selected from the group consisting of NH, O, S, CH═CH, C≡C, CO, SO₂ and OSO₂;
m is an integer from 1 to about 7;
n is an integer from 0 to about 7;
Z is selected from the group consisting of 1-, 2- or 3-pyrrolidinyl, 1-, 2-, 3- or 4-piperidinyl, 2-, 3- or 4-morpholinyl, 2-, 3- or 4-thiomorpholinyl, 1-, 2- or 3-azetidinyl, 1- or 2-piperazinyl, 2- or 3-tetrahydrofuranyl; or any above group substituted on at least one available ring atom by an alkyl group; or any above group substituted on at least one available ring nitrogen atom by a benzyl group, a substituted benzyl group, an alkoxybenzyl group, a substituted alkoxybenzyl group, a benzhydryl group or a substituted benzhydryl group; and wherein the connecting point between the —(CH₂)ₙ— group and the Z group can be any available ring carbon atom or any available ring nitrogen atom; or
R2 is —(CH₂)ₘ-Q₁-(CH₂)ₙ—Z;
Q₁ is selected from the group consisting of NH, O, S, CH═CH, C≡C, CO, SO₂ and OSO₂;
m is an integer from 1 to about 7;
n is an integer from 0 to about 7; and
Z is selected from the group consisting of 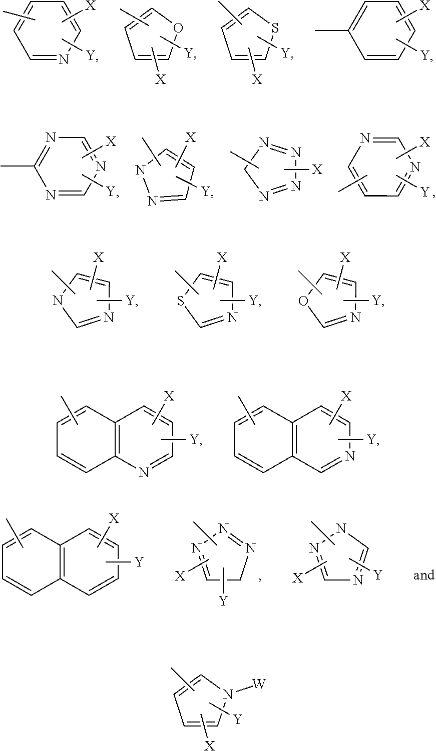
wherein each of X and Y is independently selected from the group consisting of H, halogen, CF₃, CF₂H, N₃, NCS, CN, NO₂, NX₁X₂, OX₃, SX₃, OAc, OSO₂X₃, O-acyl, S-acyl, SO₂-alkyl, SO-alkyl, SC(CH₃)₂COOX₈, OC(CH₃)₂COOX₈, C(CH₃)₂COOX₈, Si(alkyl)₃, alkyl-CN, O-aroyl, O(CH₂)ⱼOX₃, O(CH₂)ⱼNX₁X₂, NH-acyl, NH-aroyl, CHO, C(halogen)₃, COOX₃, SO₃H, SO₂NX₁X₂, CONX₁X₂, NHC(O)O-alkyl, NHSO2-alkyl, alkoxy, alkyl, alcohol, alkylmercapto, alkylamino, di-alkylamino, alkylsulfinyl or alkylsulfonyl, CX₄X₅X₆, —CH═CHX₈, and —C≡CX₈;
each of X₁ and X₂ is independently H or alkyl, or
X₁ and X₂ together form part of a heterocyclic ring having about 4 to about 7 ring members and optionally one additional heteroatom selected from O, N and S, or
X₁ and X₂ together form part of an imide ring having about 5 to about 6 members,
X₃ is selected from the group consisting of H, alkyl, aryl, NO₂, (CH₂)ₘCN, hydroxyloweralkyl, and alkyl-NX₁X₂,
each of X₄, X₅, and X₆ is independently selected from the group consisting of H, alkyl, carbocyclic ring, hydroxyloweralkyl, alkyl-OH, halogen, CN, SNO, S(SO₂)alkyl, NX₁X₂, COOX₃, CONX₃, OX₇, and O-alkyl-X₇ wherein
X₇ is selected from the group consisting of H, alkyl, NO₂, NO, P(O)(OX₈)₂, PH(O)(OX₈), S(O)ₖN(alkyl)₂, S(O)ₖX₈, S(O)ₖOX₈, COOX₈, CONX₈, SO₃H, and COX₈, wherein
X₈ is selected from the group consisting of H, alkyl, carbocyclic ring, heterocyclic ring, aromatic ring, heteroaromatic ring, and —CX₉═CHX₁₀, wherein
each of X₉ and X₁₀ is independently H or alkyl,
wherein m is an integer from 0 to 7,
j is an integer from 0 to about 6,
k is an integer from 0 to about 2
W is H or alkyl; or
R2 is —(CH₂)ₘ-Q₁-(CH₂)ₙ—Z;
Q₁ is selected from the group consisting of NH, O, S, CH═CH, C≡C, CO, SO₂ and OSO₂;
m is an integer from 1 to about 7;
n is an integer from 0 to about 7; and
Z is selected from the group consisting of an unsaturated ring having 5 ring atoms and 0 to 2 independently selected heteroatoms as ring members fused to an unsaturated ring having 5 ring atoms and 0 to 4 independently selected heteroatoms as ring members, an unsaturated ring having 5 ring atoms and 0 to 2 independently selected heteroatoms as ring members fused to an unsaturated ring having 6 or 7 ring atoms and 0 to 4 independently selected heteroatoms as ring members or an unsaturated ring having 6 ring atoms and 0 to 4 independently selected heteroatoms as ring members fused to an unsaturated ring having 6 or 7 ring atoms and 0 to 4 independently selected heteroatoms as ring members; or
R2 is —(CH₂)ₘ-Q₁-(CH₂)ₙ—Z;
Q₁ is selected from the group consisting of NH, O, S, CH═CH, C≡C, CO, SO₂ and OSO₂;
m is an integer from 1 to about 7;
n is an integer from 0 to about 7; and
Z is selected from the group consisting of 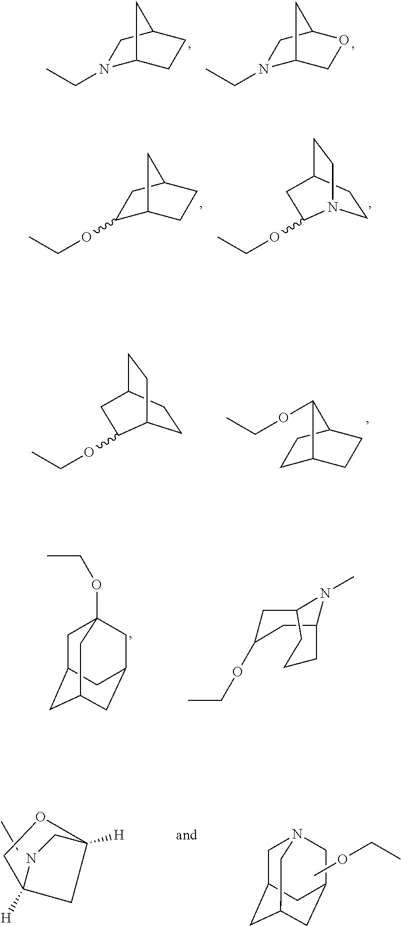
R2 is -T-(CH₂)ₙ—Z;
n is an integer from 0 to about 7;
T is selected from the group consisting of a carbocyclic ring having 3 to about 8 ring members, an unsaturated ring having 3 to about 8 carbon atoms as ring members, an aromatic ring having 5 to about 8 carbon atoms as ring members, a heterocyclic ring having 3 to about 8 ring members, a heteroaromatic ring having 5 to about 8 ring members, a bicyclic ring, a heterobicyclic ring, a tricyclic ring, a heterotricyclic ring, a polycyclic ring and a heteropolycyclic ring; and
Z is selected from the group consisting of, H, halogen, CF₃, CF₂H, N₃, NCS, CN, NO₂, NX₁X₂, OX₃, SX₃, OAc, OSO₂X₃, O-acyl, S-acyl, SO₂-alkyl, SO-alkyl, SC(CH₃)₂COOX₈, OC(CH₃)₂COOX₈, C(CH₃)₂COOX₈, Si(alkyl)₃, alkyl-CN, O-aroyl, O(CH₂)ⱼOX₃, O(CH₂)ⱼNX₁X₂, NH-acyl, NH-aroyl, CHO, C(halogen)₃, COOX₃, SO₃H, SO₂NX₁X₂, CONX₁X₂, NHC(O)O-alkyl, NHSO2-alkyl, alkoxy, alkyl, alcohol, alkylmercapto, alkylamino, di-alkylamino, alkylsulfinyl or alkylsulfonyl, CX₄X₅X₆, —CH═CHX₈, and —C≡CX₈;
each of X₁ and X₂ is independently H or alkyl, or
X₁ and X₂ together form part of a heterocyclic ring having about 4 to about 7 ring members and optionally one additional heteroatom selected from O, N and S, or
X₁ and X₂ together form part of an imide ring having about 5 to about 6 members, X₃ is selected from the group consisting of H, alkyl, aryl, NO₂, (CH₂)ₘCN, hydroxyloweralkyl, or alkyl-NX₁X₂,
each of X₄, X₅, and X₆ is independently selected from the group consisting of H, alkyl, carbocyclic ring, hydroxyloweralkyl, alkyl-OH, halogen, CN, SNO, S(SO₂)alkyl, NX₁X₂, COOX₃, CONX₃, OX₇, and O-alkyl-X₇, wherein
X₇ is selected from the group consisting of H, alkyl, NO₂, NO, P(O)(OX₈)₂, PH(O)(OX₈), S(O)ₖN(alkyl)₂, S(O)ₖX₈, S(O)ₖOX₈, COOX₈, CONX₈, SO₃H, and COX₈, wherein
X₈ is selected from the group consisting of H, alkyl, carbocyclic ring, heterocyclic ring, aromatic ring, heteroaromatic ring, and —CX₉═CHX₁₀, wherein
each of X₉ and X₁₀ is independently H or alkyl, wherein
m is an integer from 0 to 7,
j is an integer from 0 to about 6,
k is an integer from 0 to about 2; or
R2 is -T-(CH₂)ₙ—Z;
n is an integer from 0 to about 7;
T is selected from the group consisting of a carbocyclic ring having 3 to about 8 ring members, an unsaturated ring having 3 to about 8 carbon atoms as ring members, an aromatic ring having 5 to about 8 carbon atoms as ring members, a heterocyclic ring having 3 to about 8 ring members, a heteroaromatic ring having 5 to about 8 ring members, a bicyclic ring, a heterobicyclic ring, a tricyclic ring, a heterotricyclic ring, a polycyclic ring and a heteropolycyclic ring; and
Z is selected from the group consisting of a carbocyclic ring having about 4 to about 7 ring members, a heterocyclic ring having about 4 to about 7 ring members, an aromatic ring having about 5 to about 7 ring members, a heteroaromatic ring having about 5 to about 7 ring members, a bicyclic ring, a heterobicyclic ring, a tricyclic ring, a heterotricyclic ring, a polycyclic ring, a heteropolycyclic ring; or any above group substituted on at least one available ring atom by an alkyl group; or any above group substituted on at least one available ring nitrogen atom by a benzyl group, a substituted benzyl group, an alkoxybenzyl group, a substituted alkoxybenzyl group, a benzhydryl group or a substituted benzhydryl group; and wherein the connecting point between the —(CH₂)ₙ— group and the Z group can be any available ring carbon atom or any available ring nitrogen atom; or
R2 is -T-(CH₂)ₙ—Z;
n is an integer from 0 to about 7;
T is selected from the group consisting of a carbocyclic ring having 3 to about 8 ring members, an unsaturated ring having 3 to about 8 carbon atoms as ring members, an aromatic ring having 5 to about 8 carbon atoms as ring members, a heterocyclic ring having 3 to about 8 ring members, a heteroaromatic ring having 5 to about 8 ring members, a bicyclic ring, a heterobicyclic ring, a tricyclic ring, a heterotricyclic ring, a polycyclic ring and a heteropolycyclic ring; and
Z is selected from the group consisting of 1-, 2- or 3-pyrrolidinyl, 1-, 2-, 3-azetidinyl, 1- or 2-piperazinyl, 2- or 3-tetrahydrofuranyl; or any above group substituted on at least one available ring atom by an alkyl group; or any above group substituted on at least one available ring nitrogen atom by a benzyl group, a substituted benzyl group, an alkoxybenzyl group, a substituted alkoxybenzyl group, a benzhydryl group or a substituted benzhydryl group; and wherein the connecting point between the —(CH₂)ₙ— group and the Z group can be any available ring carbon atom or any available ring nitrogen atom; or
R2 is -T-(CH₂)ₙ—Z;
n is an integer from 0 to about 7;
T is selected from the group consisting of a carbocyclic ring having 3 to about 8 ring members, an unsaturated ring having 3 to about 8 carbon atoms as ring members, an aromatic ring having 5 to about 8 carbon atoms as ring members, a heterocyclic ring having 3 to about 8 ring members, a heteroaromatic ring having 5 to about 8 ring members, a bicyclic ring, a heterobicyclic ring, a tricyclic ring, a heterotricyclic ring, a polycyclic ring and a heteropolycyclic ring; and
Z is selected from the group consisting of 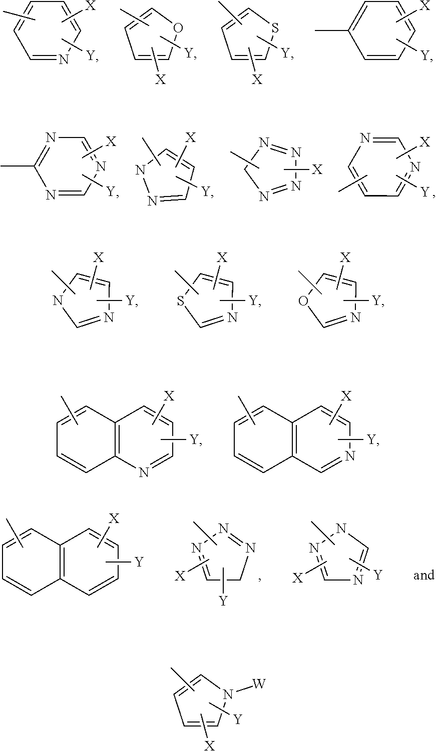
wherein each of X and Y is independently selected from the group consisting of H, halogen, CF₃, CF₂H, N₃, NCS, CN, NO₂, NX₁X₂, OX₃, SX₃, OAc, OSO₂X₃, O-acyl, S-acyl, SO₂-alkyl, SO-alkyl, SC(CH₃)₂COOX₈, OC(CH₃)₂COOX₈, C(CH₃)₂COOX₈, Si(alkyl)₃, alkyl-CN, O-aroyl, O(CH₂)ⱼOX₃, O(CH₂)ⱼNX₁X₂, NH-acyl, NH-aroyl, CHO, C(halogen)₃, COOX₃, SO₃H, SO₂NX₁X₂, CONX₁X₂, NHC(O)O-alkyl, NHSO2-alkyl, alkoxy, alkyl, alcohol, alkylmercapto, alkylamino, di-alkylamino, alkylsulfinyl or alkylsulfonyl, CX₄X₅X₆, —CH═CHX₈, and —C≡CX₈;
each of X₁ and X₂ is independently H or alkyl, or
X₁ and X₂ together form part of a heterocyclic ring having about 4 to about 7 ring members and optionally one additional heteroatom selected from O, N and S, or
X₁ and X₂ together form part of an imide ring having about 5 to about 6 members,
X₃ is selected from the group consisting of H, alkyl, aryl, NO₂, (CH₂)ₘCN, hydroxyloweralkyl, and alkyl-NX₁X₂,
each of X₄, X₅, and X₆ is independently selected from the group consisting of H, alkyl, carbocyclic ring, hydroxyloweralkyl, alkyl-OH, halogen, CN, SNO, S(SO₂)alkyl, NX₁X₂, COOX₃, CONX₃, OX₇, and O-alkyl-X₇, wherein
X₇ is selected from the group consisting of H, alkyl, NO₂, NO, P(O)(OX₈)₂, PH(O)(OX₈), S(O)ₖN(alkyl)₂, S(O)ₖX₈, S(O)ₖOX₈, COOX₈, CONX₈, SO₃H and COX₈, wherein
X₈ is selected from the group consisting of H, alkyl, carbocyclic ring, heterocyclic ring, aromatic ring, heteroaromatic ring, and —CX₉═CHX₁₀, wherein
each of X₉ and X₁₀ is independently H or alkyl, wherein
m is an integer from 0 to 7,
j is an integer from 0 to about 6,
k is an integer from 0 to about 2,
W comprises H or alkyl; or
R2 is -T-(CH₂)ₙ—Z;
n is an integer from 0 to about 7;
T is selected from the group consisting of a carbocyclic ring having 3 to about 8 ring members, an unsaturated ring having 3 to about 8 carbon atoms as ring members, an aromatic ring having 5 to about 8 carbon atoms as ring members, a heterocyclic ring having 3 to about 8 ring members, a heteroaromatic ring having 5 to about 8 ring members, a bicyclic ring, a heterobicyclic ring, a tricyclic ring, a heterotricyclic ring, a polycyclic ring and a heteropolycyclic ring; and
Z is selected from the group consisting of an unsaturated ring having 5 ring atoms and 0 to 2 independently selected heteroatoms as ring members fused to an unsaturated ring having 5 ring atoms and 0 to 4 independently selected heteroatoms as ring members, an unsaturated ring having 5 ring atoms and 0 to 2 independently selected heteroatoms as ring members fused to an unsaturated ring having 6 or 7 ring atoms and 0 to 4 independently selected heteroatoms as ring members or an unsaturated ring having 6 ring atoms and 0 to 3 independently selected heteroatoms as ring members fused to an unsaturated ring having 6 or 7 ring atoms and 0 to 4 independently selected heteroatoms as ring members; or
R2 is -T-(CH₂)ₘ-Q₁-(CH₂)ₙ—Z;
each of m and n is independently an integer from 0 to about 7;
T is selected from the group consisting of a carbocyclic ring having 3 to about 8 ring members, an unsaturated ring having 3 to about 8 carbon atoms as ring members, an aromatic ring having 5 to about 8 carbon atoms as ring members, a heterocyclic ring having 3 to about 8 ring members, a heteroaromatic ring having 5 to about 8 ring members, a bicyclic ring, a heterobicyclic ring, a tricyclic ring, a heterotricyclic ring, a polycyclic ring and a heteropolycyclic ring;
Q₁ is selected from the group consisting of NH, O, S, CH═CH, C≡C, CO, SO₂ and OSO₂; and
Z is selected from the group consisting of H, halogen, CF₃, CF₂H, N₃, NCS, CN, NO₂, NX₁X₂, OX₃, SX₃, OAc, OSO₂X₃, O-acyl, S-acyl, SO₂-alkyl, SO-alkyl, SC(CH₃)₂COOX₈, OC(CH₃)₂COOX₈, C(CH₃)₂COOX₈, Si(alkyl)₃, alkyl-CN, O-aroyl, O(CH₂)ⱼOX₃, O(CH₂)ⱼNX₁X₂, NH-acyl, NH-aroyl, CHO, C(halogen)₃, COOX₃, SO₃H, SO₂NX₁X₂, CONX₁X₂, NHC(O)O-alkyl, NHSO2-alkyl, alkoxy, alkyl, alcohol, alkylmercapto, alkylamino, di-alkylamino, alkylsulfinyl or alkylsulfonyl, CX₄X₅X₆, —CH═CHX₈, and —C≡CX₈;
each of X₁ and X₂ is independently H or alkyl, or
X₁ and X₂ together form part of a heterocyclic ring having about 4 to about 7 ring members and optionally one additional heteroatom selected from O, N and S, or
X₁ and X₂ together form part of an imide ring having about 5 to about 6 members,
X₃ is selected from the group consisting of H, alkyl, aryl, NO₂, (CH₂)ₘCN, hydroxyloweralkyl, and alkyl-NX₁X₂,
each of X₄, X₅, and X₆ is independently selected from the group consisting of H, alkyl, carbocyclic ring, hydroxyloweralkyl, alkyl-OH, halogen, CN, SNO, S(SO₂)alkyl, NX₁X₂, COOX₃, CONX₃, OX₇, and O-alkyl-X₇, wherein
X₇ comprises H, alkyl, NO₂, NO, P(O)(OX₈)₂, PH(O)(OX₈), S(O)ₖN(alkyl)₂, S(O)ₖX₈, S(O)ₖOX₈, COOX₈, CONX₈, SO₃H, and COX₈, wherein
X₈ is selected from the group consisting of a H, alkyl, carbocyclic ring, heterocyclic ring, aromatic ring, heteroaromatic ring, and —CX₉═CHX₁₀, wherein
each of X₉ and X₁₀ is independently H or alkyl, wherein
m is an integer from 0 to 7,
j is an integer from 0 to about 6,
k is an integer from 0 to about 2; or
R2 is -T-(CH₂)ₘ-Q₁-(CH₂)ₙ—Z;
each of m and n is independently an integer from 0 to about 7;
T is selected from the group consisting of a carbocyclic ring having 3 to about 8 ring members, an unsaturated ring having 3 to about 8 carbon atoms as ring members, an aromatic ring having 5 to about 8 carbon atoms as ring members, a heterocyclic ring having 3 to about 8 ring members, a heteroaromatic ring having 5 to about 8 ring members, a bicyclic ring, a heterobicyclic ring, a tricyclic ring, a heterotricyclic ring, a polycyclic ring and a heteropolycyclic ring;
Q₁ is selected from the group consisting of NH, O, S, CH═CH, C≡C, CO, SO₂ and OSO₂; and
Z is selected from the group consisting of a carbocyclic ring having about 4 to about 7 ring members, a heterocyclic ring having about 4 to about 7 ring members, an aromatic ring having about 5 to about 7 ring members, a heteroaromatic ring having about 5 to about 7 ring members, a bicyclic ring, a heterobicyclic ring, a polycyclic ring, a heteropolycyclic ring; or any above group substituted on at least one available ring atom by an alkyl group; or any above group substituted on at least one available ring nitrogen atom by a benzyl group, a substituted benzyl group, an alkoxybenzyl group, a substituted alkoxybenzyl group, a benzhydryl group or a substituted benzhydryl group; and wherein the connecting point between the —(CH₂)ₙ— group and the Z group can be any available ring carbon atom or any available ring nitrogen atom; or
R2 is -T-(CH₂)ₘ-Q₁-(CH₂)ₙ—Z;
each of m and n is independently an integer from 0 to about 7;
T is selected from the group consisting of a carbocyclic ring having 3 to about 8 ring members, an unsaturated ring having 3 to about 8 carbon atoms as ring members, an aromatic ring having 5 to about 8 carbon atoms as ring members, a heterocyclic ring having 3 to about 8 ring members, a heteroaromatic ring having 5 to about 8 ring members, a bicyclic ring, a heterobicyclic ring, a tricyclic ring, a heterotricyclic ring, a polycyclic ring and a heteropolycyclic ring;
Q₁ is selected from the group consisting of NH, O, S, CH═CH, C≡C, CO, SO₂ and OSO₂; and
Z is selected from the group consisting of 1-, 2- or 3-pyrrolidinyl, 1-, 2-, 3- or 4-piperidinyl, 2-, 3- or 4-morpholinyl, 2-, 3- or 4-thiomorpholinyl, 1-, 2- or 3-azetidinyl, 1- or 2-piperazinyl, 2- or 3-tetrahydrofuranyl; or any above group substituted on at least one available ring atom by an alkyl group; or any above group substituted on at least one available ring nitrogen atom by a benzyl group, a substituted benzyl group, an alkoxybenzyl group, a substituted alkoxybenzyl group, a benzhydryl group or a substituted benzhydryl group; and wherein the connecting point between the —(CH₂)ₙ— group and the Z group can be any available ring carbon atom or any available ring nitrogen atom; or
R2 is -T-(CH₂)ₘ-Q₁-(CH₂)ₙ—Z;
each of m and n is independently an integer from 0 to about 7;
T is selected from the group consisting of a carbocyclic ring having 3 to about 8 ring members, an unsaturated ring having 3 to about 8 carbon atoms as ring members, an aromatic ring having 5 to about 8 carbon atoms as ring members, a heterocyclic ring having 3 to about 8 ring members, a heteroaromatic ring having 5 to about 8 ring members, a bicyclic ring, a heterobicyclic ring, a tricyclic ring, a heterotricyclic ring, a polycyclic ring or a heteropolycyclic ring;
Q₁ is selected from the group consisting of N, O, S, CH═CH, C≡C, CO, SO₂ and OSO₂; and
Z is selected from the group consisting of 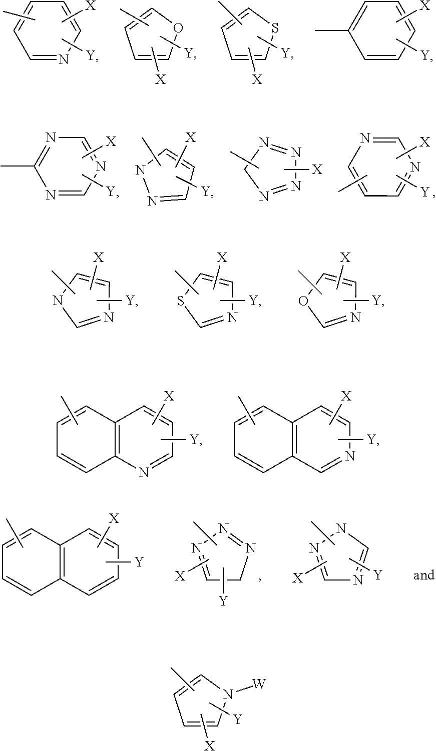
wherein each of X and Y is independently selected from the group consisting of H, halogen, CF₃, CF₂H, N₃, NCS, CN, NO₂, NX₁X₂, OX₃, SX₃, OAc, OSO₂X₃, O-acyl, S-acyl, SO₂-alkyl, SO-alkyl, SC(CH₃)₂COOX₈, OC(CH₃)₂COOX₈, C(CH₃)₂COOX₈, Si(alkyl)₃, alkyl-CN, O-aroyl, O(CH₂)ⱼOX₃, O(CH₂)ⱼNX₁X₂, NH-acyl, NH-aroyl, CHO, C(halogen)₃, COOX₃, SO₃H, SO₂NX₁X₂, CONX₁X₂, NHC(O)O-alkyl, NHSO2-alkyl, alkoxy, alkyl, alcohol, alkylmercapto, alkylamino, di-alkylamino, alkylsulfinyl or alkylsulfonyl, CX₄X₅X₆, —CH═CHX₈, and —C≡CX₈;
each of X₁ and X₂ is independently H or alkyl, or
X₁ and X₂ together form part of a heterocyclic ring having about 4 to about 7 ring members and optionally one additional heteroatom selected from O, N and S, or
X₁ and X₂ together form part of an imide ring having about 5 to about 6 members,
X₃ is selected from the group consisting of H, alkyl, aryl, NO₂, (CH₂)ₘCN, hydroxyloweralkyl, and alkyl-NX₁X₂,
each of X₄, X₅, and X₆ is independently selected from the group consisting of H, alkyl, carbocyclic ring, hydroxyloweralkyl, alkyl-OH, halogen, CN, SNO, S(SO₂)alkyl, NX₁X₂, COOX₃, CONX₃, OX₇, and O-alkyl-X₇, wherein
X₇ is selected from the group consisting of H, alkyl, NO₂, NO, P(O)(OX₈)₂, PH(O)(OX₈), S(O)ₖN(alkyl)₂, S(O)ₖX₈, S(O)ₖOX₈, COOX₈, CONX₈, SO₃H, and COX₈, wherein
X₈ is selected from the group consisting of H, alkyl, carbocyclic ring, heterocyclic ring, aromatic ring, heteroaromatic ring, and —CX₉═CHX₁₀, wherein
each of X₉ and X₁₀ is independently H or alkyl, wherein
m is an integer from 0 to 7,
j is an integer from 0 to about 6,
k is an integer from 0 to about 2,
W is H or alkyl; or
R2 is -T-(CH₂)ₘ-Q₁-(CH₂)ₙ—Z;
each of m and n is independently an integer from 0 to about 7;
T is selected from the group consisting of a carbocyclic ring having 3 to about 8 ring members, an unsaturated ring having 3 to about 8 carbon atoms as ring members, an aromatic ring having 5 to about 8 carbon atoms as ring members, a heterocyclic ring having 3 to about 8 ring members, a heteroaromatic ring having 5 to about 8 ring members, a bicyclic ring, a heterobicyclic ring, a tricyclic ring, a heterotricyclic ring, a polycyclic ring and a heteropolycyclic ring;
Q₁ is selected from the group consisting of NH, O, S, CH═CH, C≡C, CO, SO₂ and OSO₂; and
Z is selected from the group consisting of an unsaturated ring having 5 ring atoms and 0 to 2 independently selected heteroatoms as ring members fused to an unsaturated ring having 5 ring atoms and 0 to 2 independently selected heteroatoms as ring members, an unsaturated ring having 5 ring atoms and 0 to 2 independently selected heteroatoms as ring members fused to an unsaturated ring having 6 or 7 ring atoms and 0 to 4 independently selected heteroatoms as ring members or an unsaturated ring having 6 ring atoms and 0 to 3 independently selected heteroatoms as ring members fused to an unsaturated ring having 6 or 7 ring atoms and 0 to 4 independently selected heteroatoms as ring members, or R2 is -T-(CH₂)ₘ-Q₁-(CH₂)ₙ—Z;
T is selected from the group consisting of a carbocyclic ring having 3 to about 8 ring members, an unsaturated ring having 3 to about 8 carbon atoms as ring members, an aromatic ring having 5 to about 8 carbon atoms as ring members, a heterocyclic ring having 3 to about 8 ring members, a heteroaromatic ring having 5 to about 8 ring members, a bicyclic ring, a heterobicyclic ring, a tricyclic ring, a heterotricyclic ring, a polycyclic ring and a heteropolycyclic ring;
each of m and n is independently an integer from 0 to about 7;
Q₁ is selected from the group consisting of NH, O, S, CH═CH, C≡C, CO, SO₂ and OSO₂; and
Z is 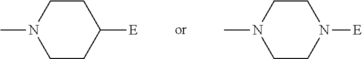
wherein E is selected from the group consisting of a C1 to about C4, linear or branched alkyl group, a phenyl group, a substituted phenyl group, a benzyl group and a substituted benzyl group; or
R2 is -T-(CH₂)ₘ-Q₁-(CH₂)ₙ—Z;
T is selected from the group consisting of a carbocyclic ring having 3 to about 8 ring members, an unsaturated ring having 3 to about 8 carbon atoms as ring members, an aromatic ring having 5 to about 8 carbon atoms as ring members, a heterocyclic ring having 3 to about 8 ring members, a heteroaromatic ring having 5 to about 8 ring members, a bicyclic ring, a heterobicyclic ring, a tricyclic ring, a heterotricyclic ring, a polycyclic ring and a heteropolycyclic ring;
each of m and n is independently an integer from 0 to about 7;
Q₁ is selected from the group consisting of NH, O, S, CH═CH, C≡C, CO, SO₂ and OSO₂;
Z is selected from the group consisting of 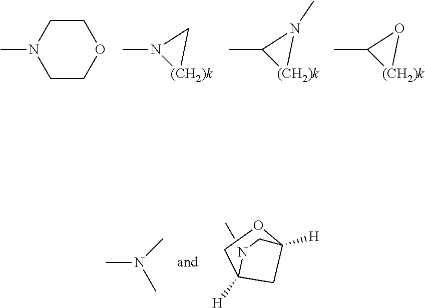
k is an integer from 1 to about 5,
each of A₁ and A₂ is independently selected from the group consisting of a C1 to about C4 alkyl group, a phenyl group and a substituted phenyl group;
R3 is 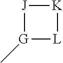
wherein G is selected from the group consisting of CH, C(CH₃), C(CN) and N,
each of L, K and J is independently selected from the group consisting of (CH₂)ₙ, (CH₃)₂, C═O, O, —CHOH, C(CH₃)OM₁, C(CH₂)ₙ(X)Y, NM₁, SO₂ SO and S,
and at least one of L, K or J is SO₂,
n is an integer from 0 to about 7;
M₁ is selected from the group consisting of H, alkyl, and C(O) M₂, wherein
M₂ is selected from the group consisting of H, alkyl, NM₃M₄, and OM₅, and each of M₃, M₄ and M₅ is independently H, OH or alkyl, and
each of X and Y is independently selected from the group consisting of H, halogen, CF₃, CF₂H, N₃, NCS, CN, NO₂, NX₁X₂, OX₃, SX₃, OAc, OSO₂X₃, O-acyl, S-acyl, SO₂-alkyl, SO-alkyl, SC(CH₃)₂COOX₈, OC(CH₃)₂COOX₈, C(CH₃)₂COOX₈, Si(alkyl)₃, alkyl-CN, O-aroyl, O(CH₂)ⱼOX₃, O(CH₂)ⱼNX₁X₂, NH-acyl, NH-aroyl, CHO, C(halogen)₃, COOX₃, SO₃H, SO₂NX₁X₂, CONX₁X₂, NHC(O)O-alkyl, NHSO2-alkyl, alkoxy, alkyl, alcohol, alkylmercapto, alkylamino, di-alkylamino, alkylsulfinyl or alkylsulfonyl, CX₄X₅X₆, —CH═CHX₈, and —C≡CX₈;
each of X₁ and X₂ is independently H or alkyl, or
X₁ and X₂ together form part of a heterocyclic ring having about 4 to about 7 ring members and optionally one additional heteroatom selected from O, N and S, or
X₁ and X₂ together form part of an imide ring having about 5 to about 6 members,
X₃ is selected from the group consisting of H, alkyl, NO₂, (CH₂)ₘCN, hydroxyloweralkyl, and alkyl-NX₁X₂,
each of X₄, X₅, and X₆ is independently selected from the group consisting of H, alkyl, carbocyclic ring, hydroxyloweralkyl, alkyl-OH, halogen, CN, SNO, S(SO₂)alkyl, NX₁X₂, COOX₃, CONX₃, OX₇, and O-alkyl-X₇, wherein
X₇ is selected from the group consisting of H, alkyl, NO₂, NO, P(O)(OX₈)₂, PH(O)(OX₈), S(O)ₖN(alkyl)₂, S(O)ₖX₈, S(O)ₖOX₈, COOX₈, CONX₈, SO₃H, and COX₈, wherein
X₈ is selected from the group consisting of H, alkyl, carbocyclic ring, heterocyclic ring, aromatic ring, heteroaromatic ring, and —CX₉═CHX₁₀, wherein
each of X₉ and X₁₀ is independently H or alkyl,
wherein m is an integer from 0 to 7,
j is an integer from 0 to about 6,
k is an integer from 0 to about 2; or
R4 is selected from the group consisting of H, halogen, CF₃, CF₂H, N₃, NCS, CN, NO₂, NX₁X₂, OH, ONO₂, SX₃, OAc, OSO₂X₃, O-acyl, S-acyl, SO₂-alkyl, SO-alkyl, SC(CH₃)₂COOX₈, OC(CH₃)₂COOX₈, C(CH₃)₂COOX₈, Si(alkyl)₃, alkyl-CN, O-aroyl, O(CH₂)ⱼOX₃, O(CH₂)ⱼNX₁X₂, NH-acyl, NH-aroyl, CHO, C(halogen)₃, COOX₃, SO₃H, SO₂NX₁X₂, CONX₁X₂, NHC(O)O-alkyl, NHSO2-alkyl, alkoxy, alkyl, alcohol, alkylmercapto, alkylamino, di-alkylamino, alkylsulfinyl or alkylsulfonyl, CX₄X₅X₆, —CH═CHX₈, and —C≡CX₈;
each of X₁ and X₂ is independently H or alkyl, or
X₁ and X₂ together form part of a heterocyclic ring having about 4 to about 7 ring members and optionally one additional heteroatom selected from O, N and S, or
X₁ and X₂ together form part of an imide ring having about 5 to about 6 members,
X₃ is selected from the group consisting of H, alkyl, NO₂, (CH₂)ₘCN, hydroxyloweralkyl, and alkyl-NX₁X₂,
each of X₄, X₅, and X₆ is independently selected from the group consisting of H, alkyl, carbocyclic ring, hydroxyloweralkyl, alkyl-OH, halogen, CN, SNO, S(SO₂)alkyl, NX₁X₂, COOX₃, CONX₃, OX₇, and O-alkyl-X₇, wherein
X₇ is selected from the group consisting of H, alkyl, NO₂, NO, P(O)(OX₈)₂, PH(O)(OX₈), S(O)ₖN(alkyl)₂, S(O)ₖX₈, S(O)ₖOX₈, COOX₈, CONX₈, SO₃H and COX₈, wherein
X₈ is selected from the group consisting of H, alkyl, carbocyclic ring, heterocyclic ring, aromatic ring, heteroaromatic ring, and —CX₉═CHX₁₀, wherein
each of X₉ and X₁₀ is independently H or alkyl, wherein
m is an integer from 0 to 7,
j is an integer from 0 to about 6,
k is an integer from 0 to about 2; or
R4 is selected from the group consisting of a carbocyclic ring having about 4 to about 7 ring members, a heterocyclic ring having about 4 to about 7 ring members, an aromatic ring having about 5 to about 7 ring members, a heteroaromatic ring having about 5 to about 7 ring members, a bicyclic ring, a heterobicyclic ring, a tricyclic ring, a heterotricyclic ring, a polycyclic ring and a heteropolycyclic ring; or
R4 is selected from the group consisting of 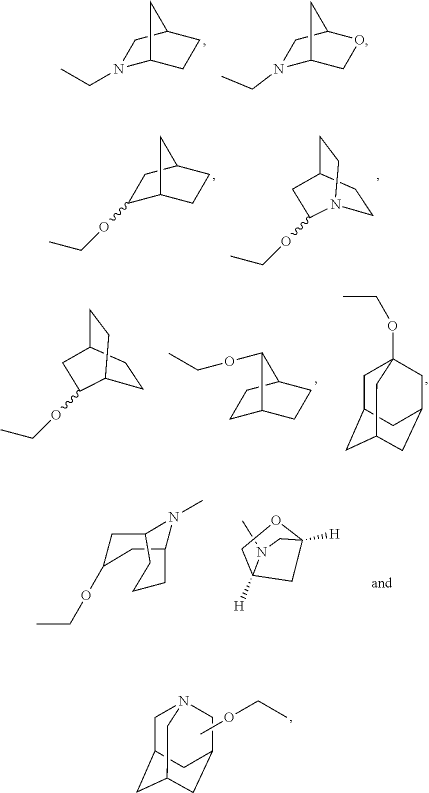
or
R4 is —(CH₂)_{d}—Z;
d is an integer from 1 to about 6;
Z is selected from the group consisting of H, halogen, CF₃, CF₂H, N₃, NCS, CN, NO₂, NX₁X₂, OX₃, SX₃, OAc, OSO₂X₃, O-acyl, S-acyl, SO₂-alkyl, SO-alkyl, SC(CH₃)₂COOX₈, OC(CH₃)₂COOX₈, C(CH₃)₂COOX₈, Si(alkyl)₃, alkyl-CN, O-aroyl, O(CH₂)ⱼOX₃, O(CH₂)ⱼNX₁X₂, NH-acyl, NH-aroyl, CHO, C(halogen)₃, COOX₃, SO₃H, SO₂NX₁X₂, CONX₁X₂, NHC(O)O-alkyl, NHSO2-alkyl, alkoxy, alkyl, alcohol, alkylmercapto, alkylamino, di-alkylamino, alkylsulfinyl or alkylsulfonyl, CX₄X₅X₆, —CH═CHX₈, and —C≡CX₈;
each of X₁ and X₂ is independently H or alkyl, or
X₁ and X₂ together form part of a heterocyclic ring having about 4 to about 7 ring members and optionally one additional heteroatom selected from O, N and S, or
X₁ and X₂ together form part of an imide ring having about 5 to about 6 members, X₃ is selected from the group consisting of H, alkyl, NO₂, (CH₂)ₘCN, hydroxyloweralkyl, and alkyl-NX₁X₂,
each of X₄, X₅, and X₆ is independently selected from the group consisting of H, alkyl, carbocyclic ring, hydroxyloweralkyl, alkyl-OH, halogen, CN, SNO, S(SO₂)alkyl, NX₁X₂, COOX₃, CONX₃, OX₇, and O-alkyl-X₇, wherein
X₇ is selected from the group consisting of H, alkyl, NO₂, NO, P(O)(OX₈)₂, PH(O)(OX₈), S(O)ₖN(alkyl)₂, S(O)ₖX₈, S(O)ₖOX₈, COOX₈, CONX₈, SO₃H, and COX₈, wherein
X₈ is selected from the group consisting of H, alkyl, carbocyclic ring, heterocyclic ring, aromatic ring, heteroaromatic ring, and —CX₉═CHX₁₀, wherein
each of X₉ and X₁₀ is independently H or alkyl,
wherein m is an integer from 0 to 7,
j is an integer from 0 to about 6,
k is an integer from 0 to about 2; or
R4 is —(CH₂)_{d}—Z;
d is an integer from 1 to about 6; and
Z is selected from the group consisting of a carbocyclic ring having about 4 to about 7 ring members, a heterocyclic ring having about 4 to about 7 ring members, an aromatic ring having about 5 to about 7 ring members, a heteroaromatic ring having about 5 to about 7 ring members, a bicyclic ring, a heterobicyclic ring, a tricyclic ring, a heterotricyclic ring, a polycyclic ring, a heteropolycyclic ring; or any above group substituted on at least one available ring atom by an alkyl group; or any above group substituted on at least one available ring nitrogen atom by a benzyl group, a substituted benzyl group, an alkoxybenzyl group, a substituted alkoxybenzyl group, a benzhydryl group or a substituted benzhydryl group; and wherein the connecting point between the —(CH₂)_{d}— group and the Z group can be any available ring carbon atom or any available ring nitrogen atom; or
R4 is —(CH₂)_{d}—Z;
d is an integer from 1 to about 6; and
Z is selected from the group consisting of 1-, 2- or 3-pyrrolidinyl, 1-, 2-, 3- or 4-piperidinyl, 2-, 3- or 4-morpholinyl, 2-, 3- or 4-thiomorpholinyl, 1-, 2- or 3-azetidinyl, 1- or 2-piperazinyl, 2- or 3-tetrahydrofuranyl; or any above group substituted on at least one ring atom by an alkyl group; or any above group substituted on at least one available ring nitrogen atom by a benzyl group, a substituted benzyl group, an alkoxybenzyl group, a substituted alkoxybenzyl group, a benzhydryl group or a substituted benzhydryl group; and wherein the connecting point between the —(CH₂)_{d}— group and the Z group can be any available ring carbon atom or any available ring nitrogen atom; or
R4 is —(CH₂)ₘ-Q₁-(CH₂)ₙ—Z;
Q₁ is selected from the group consisting of NH, O, S, CH═CH, C≡C, CO, SO₂ and OSO₂;
m is an integer from 1 to about 7;
n is an integer from 0 to about 7; and
Z is selected from the group consisting of H, halogen, CF₃, CF₂H, N₃, NCS, CN, NO₂, NX₁X₂, OX₃, SX₃, OAc, OSO₂X₃, O-acyl, S-acyl, SO₂-alkyl, SO-alkyl, SC(CH₃)₂COOX₈, OC(CH₃)₂COOX₈, C(CH₃)₂COOX₈, Si(alkyl)₃, alkyl-CN, O-aroyl, O(CH₂)ⱼOX₃, O(CH₂)ⱼNX₁X₂, NH-acyl, NH-aroyl, CHO, C(halogen)₃, COOX₃, SO₃H, SO₂NX₁X₂, CONX₁X₂, NHC(O)O-alkyl, NHSO2-alkyl, alkoxy, alkyl, alcohol, alkylmercapto, alkylamino, di-alkylamino, alkylsulfinyl or alkylsulfonyl, CX₄X₅X₆, —CH═CHX₈, and —C≡CX₈;
each of X₁ and X₂ is independently H or alkyl, or
X₁ and X₂ together form part of a heterocyclic ring having about 4 to about 7 ring members and optionally one additional heteroatom selected from O, N and S, or
X₁ and X₂ together form part of an imide ring having about 5 to about 6 members,
X₃ is selected from the group consisting of H, alkyl, NO₂, (CH₂)ₘCN, hydroxyloweralkyl, and alkyl-NX₁X₂,
each of X₄, X₅, and X₆ is independently selected from the group consisting of H, alkyl, carbocyclic ring, hydroxyloweralkyl, alkyl-OH, halogen, CN, SNO, S(SO₂)alkyl, NX₁X₂, COOX₃, CONX₃, OX₇, and O-alkyl-X₇, wherein
X₇ is selected from the group consisting of H, alkyl, NO₂, NO, P(O)(OX₈)₂, PH(O)(OX₈), S(O)ₖN(alkyl)₂, S(O)ₖX₈, S(O)ₖOX₈, COOX₈, CONX₈, SO₃H, and COX₈, wherein
X₈ is selected from the group consisting of H, alkyl, carbocyclic ring, heterocyclic ring, aromatic ring, heteroaromatic ring, and —CX₉═CHX₁₀, wherein
each of X₉ and X₁₀ is independently H or alkyl,
wherein m is an integer from 0 to 7,
j is an integer from 0 to about 6, or
k is an integer from 0 to about 2; or
R4 is —(CH₂)ₘ-Q₁-(CH₂)ₙ—Z;
Q₁ is selected from the group consisting of NH, O, S, CH═CH, C≡C, CO, SO₂ and OSO₂;
m is an integer from 1 to about 7;
n is an integer from 0 to about 7; and
Z is selected from the group consisting of a carbocyclic ring having about 4 to about 7 ring members, a heterocyclic ring having about 4 to about 7 ring members, an aromatic ring having about 5 to about 7 ring members, a heteroaromatic ring having about 5 to about 7 ring members; a bicyclic ring, a heterobicyclic ring, a tricyclic ring, a heterotricyclic ring, a polycyclic ring, a heteropolycyclic ring; or any above group substituted on at least one available ring atom by an alkyl group; or any above group substituted on at least one available ring nitrogen atom by a benzyl group, a substituted benzyl group, an alkoxybenzyl group, a substituted alkoxybenzyl group, a benzhydryl group or a substituted benzhydryl group; and wherein the connecting point between the —(CH₂)ₙ— group and the Z group can be any available ring carbon atom or any available ring nitrogen atom; or
R4 is —(CH₂)ₘ-Q₁-(CH₂)ₙ—Z;
Q₁ is selected from the group consisting of NH, O, S, CH═CH, C≡C, CO, SO₂ and OSO₂;
m is an integer from 1 to about 7;
n is an integer from 0 to about 7; and
Z is selected from the group consisting of 1-, 2- or 3-pyrrolidinyl, 1-, 2-, 3- or 4-piperidinyl, 2-, 3- or 4-morpholinyl, 2-, 3- or 4-thiomorpholinyl, 1-, 2- or 3-azetidinyl, 1- or 2-piperazinyl, 2- or 3-tetrahydrofuranyl; or any above group substituted on at least one available ring atom by an alkyl; or any above group substituted on at least one available ring nitrogen atom by a benzyl group, a substituted benzyl group, an alkoxybenzyl group, a substituted alkoxybenzyl group, a benzhydryl group or a substituted benzhydryl group; and wherein the connecting point between the —(CH₂)ₙ— group and the Z group can be any available ring carbon atom or any available ring nitrogen atom; or
R4 is —(CH₂)ₘ-Q₁-(CH₂)ₙ—Z;
Q₁ is selected from the group consisting of NH, O, S, CH═CH, C≡C, CO, SO₂ and OSO₂;
m is an integer from 1 to about 7;
n is an integer from 0 to about 7; and
Z is selected from the group consisting of 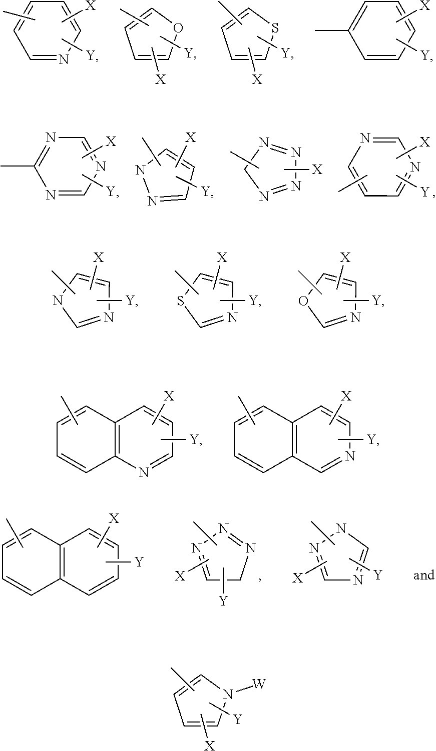
wherein each of X and Y is independently selected from the group consisting of H, halogen, CF₃, CF₂H, N₃, NCS, CN, NO₂, NX₁X₂, OX₃, SX₃, OAc, OSO₂X₃, O-acyl, S-acyl, SO₂-alkyl, SO-alkyl, SC(CH₃)₂COOX₈, OC(CH₃)₂COOX₈, C(CH₃)₂COOX₈, Si(alkyl)₃, alkyl-CN, O-aroyl, O(CH₂)ⱼOX₃, O(CH₂)ⱼNX₁X₂, NH-acyl, NH-aroyl, CHO, C(halogen)₃, COOX₃, SO₃H, SO₂NX₁X₂, CONX₁X₂, NHC(O)O-alkyl, NHSO2-alkyl, alkoxy, alkyl, alcohol, alkylmercapto, alkylamino, di-alkylamino, alkylsulfinyl or alkylsulfonyl, CX₄X₅X₆, —CH═CHX₈, and —C≡CX₈;
each of X₁ and X₂ is independently H or alkyl, or
X₁ and X₂ together form part of a heterocyclic ring having about 4 to about 7 ring members and optionally one additional heteroatom selected from O, N and S, or
X₁ and X₂ together form part of an imide ring having about 5 to about 6 members,
X₃ is selected from the group consisting of H, alkyl, NO₂, (CH₂)ₘCN, hydroxyloweralkyl, and alkyl-NX₁X₂,
each of X₄, X₅, and X₆ is independently selected from the group consisting of H, alkyl, carbocyclic ring, hydroxyloweralkyl, alkyl-OH, halogen, CN, SNO, S(SO₂)alkyl, NX₁X₂, COOX₃, CONX₃, OX₇, and O-alkyl-X₇, wherein
X₇ is selected from the group consisting of H, alkyl, NO₂, NO, P(O)(OX₈)₂, PH(O)(OX₈), S(O)ₖN(alkyl)₂, S(O)ₖX₈, S(O)ₖOX₈, COOX₈, CONX₈, SO₃H and COX₈, wherein
X₈ is selected from the group consisting of H, alkyl, carbocyclic ring, heterocyclic ring, aromatic ring, heteroaromatic ring, and —CX₉═CHX₁₀, wherein
each of X₉ and X₁₀ is independently H or alkyl,
m is an integer from 0 to 7,
j is an integer from 0 to about 6,
k is an integer from 0 to about 2, and
W is H or alkyl.

2. The pharmaceutical composition of claim 1, wherein the polymer is selected from the group consisting of ionizable cellulosic polymers, non-ionizable cellulosic polymers, and vinyl polymers and copolymers.

3. The pharmaceutical composition of claim 1 wherein the polymer is a water-soluble polymer or an enteric polymer or both.

4. The pharmaceutical composition of claim 1, wherein the polymer is a gastric-soluble polymer.

5. The pharmaceutical composition of claim 1, wherein the polymer is a pH-sensitive polymer.

6. The pharmaceutical composition of claim 1, wherein the polymer is a water soluble polymer.

7. The pharmaceutical composition of claim 1, wherein the polymer is an ionic or nonionic polymer.

8. The pharmaceutical composition of claim 1, wherein the polymer is selected from HPMCP HP 55, HPMC E15, HPMC 100Cp, HPMC E4M, HPC Klucel, HPMC E5, Affinisol HPMCAS 716 (L), Affinisol HPMCAS 912 (M), Affinisol HPMCAS 126 (H), Kollidon VA64, PVP K30, Eudragit S100, Eudragit L100, Eudragit L100-55, Soluplus, ethyl cellulose, PEG 6000, PEG 1500, Poloxamer 188, Poloxamer 407, HPMCAS-L, HPMCAS-H, HPC-SSL, HPC-SL, Kollidon VA64, Kollidon K30, HPMCAS MMP, Gelucire 50/13, Compritol 888 ATO, Eudragit E PO, Ethocel Standard 10, Eudragit E PP, Eudragit E PO and combinations thereof.

9. The pharmaceutical composition of claim 1, wherein a surfactant is present and is selected from the group consisting of anionic surfactants and nonionic surfactants.

10. The pharmaceutical composition of claim 1, wherein a surfactant or surfactant-like material is present and is a homogeneous, amorphous or semi-crystalline powder.

11. The pharmaceutical composition of claim 1, wherein a surfactant or surfactant-like material is present and is non-ionic and is water soluble.

12. The pharmaceutical composition of claim 1, wherein a surfactant or surfactant-like material is present and is anionic.

13-17. (canceled)

18. The pharmaceutical composition of claim 1, wherein the compound is selected from

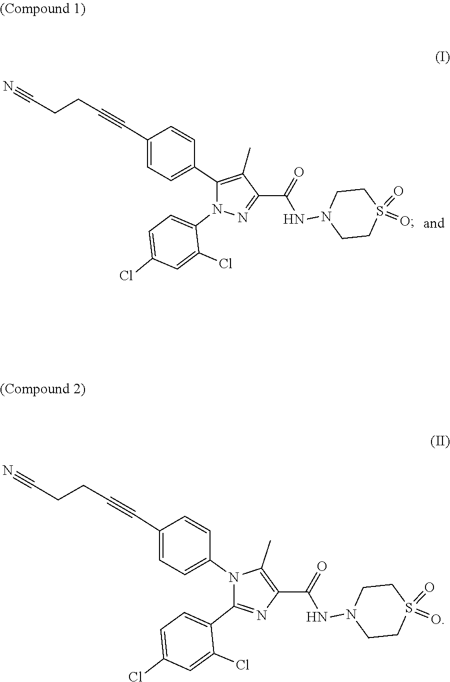 [SMILES: Cc1c(C(=O)NN2CCS(=O)(=O)CC2)nn(-c2ccc(Cl)cc2Cl)c1-c1ccc(C#CCCC#N)cc1 ; Cc1c(C(=O)NN2CCS(=O)(=O)CC2)nc(-c2ccc(Cl)cc2Cl)n1-c1ccc(C#CCCC#N)cc1]

19. A process of making the composition of claim 1, comprising spray drying a mixture comprising the compound, the one or more polymers, the one or more surfactants or surfactant-like materials, if present, the one or more carriers, if present, the one or more excipients, if present, and the one or more solvents, if present.

20-25. (canceled)

26. The pharmaceutical composition of claim 25, wherein a surfactant is present and is selected from one of polyethylene glycol-glyceryl triricinoleate (PEG-GTR), Labrasol® ALF, Gelucire® 44/14, Labrafil® M1944, Capryol® 90, Kolliphor RH40, Poloxamer P407, SLS, D-α-Tocopheryl polyethylene glycol 1000 succinate, and Tween 80.

27. The pharmaceutical composition of claim 1, wherein a surfactant is present and is selected from one of polyethylene glycol-glyceryl triricinoleate (PEG-GTR), Labrasol® ALF, Gelucire® 44/14, Labrafil® M1944, Capryol® 90, Kolliphor RH40, Poloxamer P407, SLS, D-α-Tocopheryl polyethylene glycol 1000 succinate, and Tween 80.

28. The pharmaceutical composition of claim 1, wherein
the compound is 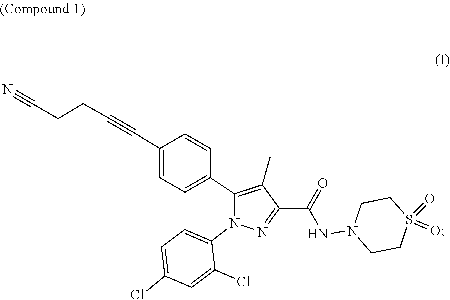 [SMILES: Cc1c(C(=O)NN2CCS(=O)(=O)CC2)nn(-c2ccc(Cl)cc2Cl)c1-c1ccc(C#CCCC#N)cc1]
the polymer is selected from one of HPMCP HP 55, HPMC E15, HPMC 100Cp, HPMC E4M, HPC Klucel, HPMC E5, Affinisol HPMCAS 716 (L), Affinisol HPMCAS 912 (M), Affinisol HPMCAS 126 (H), Kollidon VA64, PVP K30, Eudragit S100, Eudragit L100, Eudragit L100-55, Soluplus, ethyl cellulose, PEG 6000, PEG 1500, Poloxamer 188, Poloxamer 407, HPMCAS-L, HPMCAS-H, HPC-SSL, HPC-SL, Kollidon VA64, Kollidon K30, HPMCAS MMP, Gelucire 50/13, Compritol 888 ATO, Eudragit E PO, Ethocel Standard 10, Eudragit E PP, and Eudragit E PO; and
a surfactant is present and is selected from one of polyethylene glycol-glyceryl triricinoleate (PEG-GTR), Labrasol® ALF, Gelucire® 44/14, Labrafil® M1944, Capryol® 90, Kolliphor RH40, Poloxamer P407, SLS, D-α-Tocopheryl polyethylene glycol 1000 succinate, and Tween 80.

29. The pharmaceutical composition of claim 1, wherein
the compound is 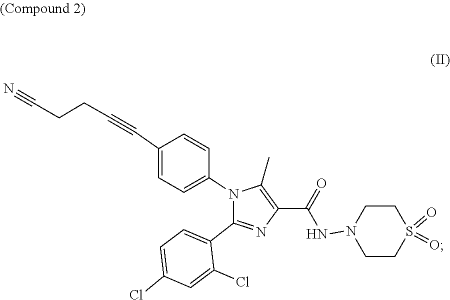 [SMILES: Cc1c(C(=O)NN2CCS(=O)(=O)CC2)nc(-c2ccc(Cl)cc2Cl)n1-c1ccc(C#CCCC#N)cc1]
the polymer is selected from one of HPMCP HP 55, HPMC E15, HPMC 100Cp, HPMC E4M, HPC Klucel, HPMC E5, Affinisol HPMCAS 716 (L), Affinisol HPMCAS 912 (M), Affinisol HPMCAS 126 (H), Kollidon VA64, PVP K30, Eudragit S100, Eudragit L100, Eudragit L100-55, Soluplus, ethyl cellulose, PEG 6000, PEG 1500, Poloxamer 188, Poloxamer 407, HPMCAS-L, HPMCAS-H, HPC-SSL, HPC-SL, Kollidon VA64, Kollidon K30, HPMCAS MMP, Gelucire 50/13, Compritol 888 ATO, Eudragit E PO, Ethocel Standard 10, Eudragit E PP, and Eudragit E PO; and
a surfactant is present and is selected from one pf polyethylene glycol-glyceryl triricinoleate (PEG-GTR), Labrasol® ALF, Gelucire® 44/14, Labrafil® M1944, Capryol® 90, Kolliphor RH40, Poloxamer P407, SLS, D-α-Tocopheryl polyethylene glycol 1000 succinate, and Tween 80.

30. (canceled)

31. A method of increasing, enhancing or improving the bioavailability of the compound of Formula I or Formula II as each is defined in claim 1, comprising mixing the compound with one or more polymers, optionally one or more surfactants or surfactant-like materials, optionally one or more carriers, optionally one or more excipients, and optionally one or more solvents.

32-46. (canceled)

47. The pharmaceutical composition of claim 1, wherein
the compound is present in a concentration of 10% or more by weight,
a surfactant or surfactant-like material is present,
and the composition has a relative concentration of polymer that is greater than the concentration of each of the compound and surfactant by weight.

48-53. (canceled)
